(19) United States
(12) Patent Application Publication
(10) Pub. No.: US 2023/0075499 A1
(43) Pub. Date: Mar. 9, 2023

(54) BISPECIFIC PROTEINS AND METHODS FOR PREPARING SAME

(71) Applicant: Ibentrus, Inc., Daejeon (KR)

(72) Inventors: Hoeon Kim, Seongnam-si (KR); Sohyun Bae, Daejeon (KR)

(21) Appl. No.: 16/317,987

(22) PCT Filed: Jul. 19, 2017
(86) PCT No.: PCT/KR2017/007791

§ 371 Date: Jan. 15, 2019

(30) Foreign Application Priority Data
Jul. 19, 2016 (KR) 10-2016-0091157

(51) Int. Cl. C07K 16/46; C07K 16/10

(52) U.S. Cl. CPC C07K 16/468; C07K 16/1018; C07K 2317/31; C07K 2317/526; C07K 2317/522

(57) ABSTRACT

The present invention relates to a bispecific protein and a method preparing the same, wherein mutation is introduced into heavy chains and/or light chains to enhance heterodimerization between a heavy chain (CH3 domain or Fc) and a heavy chain (CH3 domain or Fc) and dimerization between a heavy chain (CH1 domain) and a light chain, both targeting the same material, thereby constructing heterodimeric bispecific proteins of high purity. A bispecific protein according to the present invention can find applications in a variety of fields comprising cancer therapy, singling regulation, diagnosis, etc.

### TECHNICAL FIELD

The present invention relates to a bispecific protein with a high heterodimerization rate and a method of preparing a bispecific protein.

### BACKGROUND ART

A bispecific antibody (BsAb), which is an antibody having two paratopes capable of recognizing two different types of target antigens, is a generic term for an antibody capable of binding simultaneously to two different target antigens or for an antigen binding fragment thereof. This feature allows for the establishment of a therapeutic strategy which is impossible with conventional monoclonal antibodies. Bispecific antibodies rarely occur naturally and are in most part artificially constructed to simultaneously bind to two different types of biological targets. The double-targeting ability can provide such BsAbs with new applicable fields which have not been managed by monospecific antibodies (MsAbs). When it comes to therapeutic purposes, considerable interest is arising in (1) reliable recruitment of immune cells into the proximity of target cells, (2) inhibition or activation of two distantly separated signaling pathways in target cells to create a synergistic effect, and (3) specific and regulatory delivery of therapeutic substance, medications, toxins, etc. to target cells.

A variety of BsAb-related techniques (45 different formats) has been developed. These techniques are classified into four categories on a structural basis: first, heterologous bispecification of heavy chains by various methods comprising structural complementarity, also known as Knob-into-Hole or simply KiH, electrostatic steering effect, and CH3 domain shuffling (called SEEDbody™); second, various antibody fragment formats such as Diabody (Diabody: dimeric antibody fragments) BiTE (Bi Bi-specific T-cell engagers), and DART (DART: dual affinity retargeting bispecific antibody); third, technology using one or more functional domains combined with intact antibodies, such as Modular Antibody™, Zybody™, dAbs™ (dAbs: Single domain antibodies), and DVD-IG™ (DVD-Ig: dual-variable-domain immunoglobulin); and fourth, techniques adopting a full-length IgG-like scheme such as Duobody™ (Fab-Arm Exchange), CrossMab™, Azymetric™, and kI Body™ have been developed.

For example, U.S. Patent No. 2013-892198 A to Zymeworks discloses a heteropolymer structure of immunoglobulin chains having mutations in the Fc domain, specifically stating that antibodies of the heteropolymer structure can be constructed by modifying cysteine residues involved in disulfide bonds into charged amino acids to exert an electrical interaction.

There are problems with conventional techniques of constructing bispecific antibodies as follows.

First, there are undesirable combinations of heavy chains. When heavy chains (A and B) originating respectively from two antibodies targeting different epitopes randomly combine with each other, two combinations of the same origins (AA and BB) and one combination of different origins (AB or BA) are formed. Combinations between heavy chains from the same origins act as undesired impurities in constructing bispecific antibodies, decreasing the purity of bispecific antibodies and requiring a process of removing the combinations. Therefore, they may have disadvantageous effects, such as provoking difficulty in the isolation and purification of antibodies, inciting undesired immune responses or signal transduction to cause side effects, etc. Hence, ongoing needs exist for forming only combinations between heavy chains of heterogeneous origins allowing none or up to a considerably restricted level of combinations between heavy chains of homogeneous origin.

Second, although light and heavy chains of homogeneous origins are specifically combined with each other, cases occur in which the light and heavy chains that combine with each other are of heterogeneous origins. A bispecific antibody targets two different kinds of epitopes. Each epitope can be recognized by an antibody in which light and heavy chains of the same origin are combined. Antibodies that are created through combinations of light and heavy chains from heterogeneous origins may recognize new epitopes other than the desired two epitopes, act as an impurity making it difficult to isolate and purify antibodies of interest, and cause a side effect. Therefore, the production of bispecific antibodies requires combinations of correct pairs of light and heavy chains while a non-specific combination of light and heavy chains does not occur or occurs at up to an insignificant level.

In consideration of the above-mentioned problems with the provision of bispecific antibodies, requirements for construction of bispecific antibodies are summarized as follows. A total of ten combinations is possible when bispecific antibodies are constructed with light and heavy chains from two different kinds of antibodies respectively recognizing two different epitopes, as shown in FIG. 1. Of them, the combination marked by a dotted line circle meets the requirements that (1) heavy chains derived from different antibodies are combined with each other and (2) light and heavy chains derived from the same antibodies are coupled to each other. The other nine combinations should be not formed or should be formed at a minimum level.

WO2014142591, which is a previous patent application of the present inventor, introduces “protein in which electrical interaction is introduced within a hydrophobic interaction site and preparation method therefor”. Disclosed in the patent application is a protein or an antibody having an electrical interaction introduced in a hydrophobic interaction site thereof wherein the electrical interaction is made by a positive charge and a negative charge on a positively charged substance and a negatively charged substance which are changed from a pair of hydrophobic amino acids selected in the hydrophobic interaction site. Leading to the present invention, intensive and thorough research made by the present inventors resulted in the finding that bispecific antibodies can be formed using electrostatic interaction in a non-hydrophobic interaction site which was not taken into consideration in the previous patent application and can be constructed at high yield even by size-dependent coupling and/or amino acid change (swapping) between coupled pairs.

### PRIOR ART DOCUMENT

[Patent Document]

(Patent Document 1) Korean Patent Number 10-2014-0019385 A (Feb. 14, 2014)

### DISCLOSURE

### Technical Problem

In order to solve the above problems, the present invention provides a bispecific antibody with high purity and a method of preparing the same.

An embodiment provides a dimer comprising a first CH3 domain and a second CH3 domain of an antibody, or Fc regions comprising the CH3 domains, wherein

the first CH3 domain and the second domain are mutated such that at least one selected from among amino acid pairs forming amino acid-amino acid bonds between the first CH3 domain and the second CH3 domain is modified by at least one of the following mutations:

(1) a mutation in which, of at least one amino acid pair between the CH3 domains, one amino acid is substituted with an amino acid having a positive charge and the other is substituted with an amino acid having a negative charge;

(2) a mutation in which amino acids in at least one amino acid pair between the CH3 domains are swapped with each other; and

(3) a mutation in which, of at least one amino acid pair between the CH3 domains, one amino acid is substituted with a large hydrophobic amino acid while the other is substituted with a small hydrophobic amino acid.

The first CH3 domain and the second CH3 domain may be derived from the same or different kinds of antibodies (immunoglobulins).

Another embodiment provides a nucleic acid molecule encoding the modified CH3 domain or a modified Fc region comprising the modified CH3 domain, a recombinant vector carrying the nucleic acid molecule, and a recombinant cell containing the recombinant vector therein.

As used herein the terms “one amino acid” and “the other amino acid” in the expression “of at least one amino acid pair, one amino acid . . . and the other amino acid . . . ” mean one amino acid and the other amino acid of the two amino acids in each of one or more amino acid pairs, respectively (hereinafter the same definition will be applied).

Another embodiment provides a bispecific protein for targeting two different kinds of targets, the bispecific protein comprising a first CH3 domain or a first Fc region comprising the first CH3 domain and a second CH3 domain or a second Fc region comprising the second CH3 domain, wherein the first CH3 domain and the second CH3 domain are mutated such that at least one selected from amino acid pairs forming amino acid-amino acid bonds between the first CH3 domain and the second CH3 domain is modified by at least one of the following mutations:

(1) a mutation in which, of at least one amino acid pair between the CH3 domains, one amino acid is substituted with an amino acid having a positive charge while the other is substituted with an amino acid having a negative charge;

(2) a mutation in which amino acids in at least one amino acid pair between the CH3 domains are swapped with each other; and

(3) a mutation in which, of at least one amino acid pair between the CH3 domains, one amino acid is substituted with a large hydrophobic amino acid while the other is substituted with a small hydrophobic amino acid.

The amino acid pair that is substituted with amino acids having opposite charges in step (1) may be an amino acid under hydrophobic interaction and/or an amino acid under non-hydrophobic interaction. In one embodiment, at least one of the two amino acids constituting an amino acid pair may be not a hydrophobic amino acid (i.e., a hydrophilic amino acid). The electrostatic interaction that is introduced by step (1) of substitution with amino acids having opposite charges may contribute to improving the formation of a heterodimer between Fc regions.

As used herein, the term “heterodimer” means a fusion body in which two different proteins are coupled to each other and is intended to encompass any bispecific protein in which two proteins targeting respective different targets are coupled to each other (e.g., bispecific antibody), etc.

The amino acid switching (swapping) in step (2) may be conducted in any amino acid pair forming an amino acid-amino acid linkage in the Fc regions or CH3 domains and, for example, in at least one amino acid pair selected from all the amino acid pairs which are each bonded through interaction other than electrostatic interaction, hydrophobic interaction, and amino acid size difference-based interaction. As such, two amino acid residues that interact with each other (e.g., bond themselves to each other) in each amino acid pair (i.e., an amino acid residue of the first CH3 domain and the other amino acid of the second CH3 domain in the interacting amino acid pair) may be exchanged with each other to decrease the possibility of forming a homodimer because the presence of the same amino acid at the counterpart positions for amino acid interaction between the CH3 domains of homogeneous origins makes the bonding therebetween difficult, thereby contributing to improving a heterodimerization rate (for example, when an amino acid pair of S364 and K370 undergoes S364K mutation in the first domain and K370S mutation in the second CH3 domain, there is no interaction between the homogeneous CH3 domains because both amino acids at positions 364 and 370 become lysine (K) in the first CH3 domain and serine (S) in the second CH3 domain, but interaction occurs between the heterogeneous CH3 domain to form a heterodimer only).

The step (3) of substitution with amino acids different in size improves structural engagement suitability between a large amino acid and a small amino acid (that is, a large amino acid is inserted into a spare space established by a small amino acid, thereby increasing bonding efficiency), with the consequent increase of heterodimerization rates. Particularly, an interacting amino acid pair is mutated such that one amino acid is substituted with a large hydrophobic amino acid while the other amino acid is substituted with a small hydrophobic amino acid whereby advantage is taken of the difficulty in making a bond between large amino acids or between small amino acids to minimize a homodimerization rate (large amino acids, if existing respectively in two opposite chains, render the two chains distant from the each other to obstruct dimerization whereas two small amino acids, if existing in two opposite chains, interact with each other at low possibility because of a long distance therebetween and have difficulty in interaction therebetween). On the contrary, a large hydrophobic amino acid in one CH3 domain or Fc and a small hydrophobic amino acid in the other CH3 domain or Fc undergo hydrophobic interaction with each other at a closer distance compared to the pre-mutation amino acids, thus making a condition good for heterodimerization. Therefore, a large and a small amino acid to be substituted in the step of substitution with amino acids different in size may be both selected from among hydrophobic amino acids. For example, the large amino acid may be at least one selected from the group consisting of tryptophan and phenylalanine, which are both hydrophobic. In addition, the small amino acid may be at least one selected from the group consisting of alanine, glycine, and valine, which are all hydrophobic.

The bispecific protein comprising the mutant Fc regions or CH3 domains may be selected from among any type of proteins targeting (e.g., specifically recognizing and/or binding to) two different kinds of targets. For targeting, the bispecific protein comprising the mutant Fc regions or CH3 domains may comprise two targeting domains capable of targeting (specifically recognizing and/or binding to) two different kinds of targets, respectively (for example, a first targeting domain for targeting a first target and a second targeting domain for targeting a second target). The targeting domains may form a covalent or non-covalent bond (linkage) to the mutant Fc regions or CH3 domains, respectively, in a direct or indirect (e.g., via a linker) manner. For example, the bispecific proteins comprising the mutant Fc regions or CH3 domains may be at least one selected from the group consisting of a bispecific antibody, an antigen-binding fragment of a bispecific antibody (e.g., (scFv-Fc)2, etc.), a bispecific antibody analog (e.g., nanobody, peptibody, peptide, aptide, etc.), and a fusion protein of a target-specific binding polypeptide and the mutant Fc region or CH3 domain.

The target-specific binding polypeptide may be any polypeptide that binds specifically to a biological target substance (any compounds present in the body comprising proteins, nucleic acids, and the like) and may be at least one polypeptide selected from, for example, the group consisting of a paratope (e.g., e.g., a CDR or variable region of a heavy chain and/or a light chain), single-chain Fv (scFv), a membrane protein (e.g., various receptors, etc.), a membrane protein ectodomain, and a ligand (e.g., various growth factors, cytokines, etc.). In one embodiment, the fusion protein of a target-specific binding polypeptide and the mutant Fc region or CH3 domain may be at least one selected from the group consisting of a fusion protein of a membrane protein and the mutant Fc region or CH3 domain, a fusion protein of a membrane protein ectodomain and the mutant Fc region or CH3 domain, a fusion protein of a ligand and the mutant Fc region or CH3 domain, and a fusion protein of scFv and the mutant Fc region or CH3 domain.

When the bispecific protein comprising the mutant Fc region or CH3 domain is an antibody, an antigen-binding fragment of an antibody, or an antibody analog, the targeting domain may be a paratope (e.g., e.g., a CDR or variable region of a heavy chain and/or a light chain). For the above-mentioned fusion protein of a target-specific binding polypeptide and the mutant Fc region or CH3 domain, the target-specific binding polypeptide may be at least one selected from the group consisting of a membrane protein (e.g., various receptors), a membrane protein ectodomain, a ligand (e.g., various growth factors, cytokines, etc.), and a paratope (e.g., a CDR or variable region of a heavy chain and/or a light chain).

The two different kinds of targets may refer to two different kinds of biological substances (e.g., proteins) or different regions within one biological substance (e.g., one protein). The bispecific protein comprising the mutant Fc region or CH3 domain is characterized by an increase in heterodimerization rate, a decrease in homodimerization, and/or stability, compared to a bispecific protein comprising a non-mutant (wild-type) Fc region or CH3 domain.

Another embodiment provides a bispecific antibody or an antigen-binding fragment thereof, the bispecific antibody comprising a first CH1 domain and a first Cl (light chain constant region) domain derived respectively from the heavy chain and light chain of an antibody recognizing a first epitope and a second CH1 domain and a second CL domain derived respectively from the heavy chain and light chain of an antibody recognizing a second epitope, wherein the CH1 domains and the CL domains are mutated to contain at least one of the following mutations:

a mutation in which, of the two amino acids constituting each pair of one or more first amino acid pairs selected from among amino acid pairs forming respective amino acid-amino acid bonds between the first CH1 domain and the first CL domain, one is substituted with an amino acid having a positive charge and the other is substituted with an amino acid having a negative charge; and

a mutation in which, of the two amino acids constituting each pair of one or more second amino acid pairs selected from among amino acid pairs forming respective amino acid-amino acid bonds between the second CH1 domain and the second CL domain, one is substituted with an amino acid having a positive charge and the other is substituted with an amino acid having a negative charge.

The first epitope and the second epitope may exist in respective different proteins (antibodies) or in different (discriminative) regions of one protein (antigen).

In the bispecific antibody or the antigen-binding fragment thereof according to one embodiment, the amino acids substituted respectively in the first CH1 domain and the second CH1 domain have opposite charges, the amino acids substituted respectively in the first CL domain and the first CH1 domain have opposite charges, and the amino acids substituted respectively in the second CL domain and the second CH1 domain have opposite charges.

In the bispecific antibody or the antigen-binding fragment thereof, for example,

the amino acid, positioned in the first CH1 domain, as a member of at least one first amino acid pair selected from among amino acid pairs forming respective amino acid-amino acid bonds between the first CH1 domain and the first CL domain may be substituted with an amino acid having a positive charge while the other member positioned in the first CL domain may be substituted with an amino acid having a charge different from that of the amino acid substituted in the first CH1 domain, that is, a negative charge, and

the amino acid, positioned in the second CH1 domain, as a member of at least one second amino acid pair selected from among amino acid pairs forming respective amino acid-amino acid bonds between the second CH1 domain and the second CL domain may be substituted with an amino acid having a charge different from that of the amino acid substituted in the first CH1 domain, that is, a negative charge while the other member positioned in the second CL domain may be substituted with an amino acid having a charge different from that of the amino acid substituted in the second CH1 domain, that is, a positive charge.

In the bispecific antibody or the antigen-binding fragment thereof comprising mutant CH1 CL domains, the first and the second amino acid pair to be substituted may be the same or different, the positively charged amino acids substituted in the first and the second amino acid pair may be the same or different, and the negatively charged amino acids substituted in the first and the second amino acid pair may be the same or different. The antigen-binding fragment of the bispecific antibody comprising a mutant CH1 domain and a mutant CL domain may be, for example, a F(ab′)2 fragment. The bispecific antibody comprising mutant CH1 and CL domains or the antigen-binding fragment thereof targets the same epitopes as those for a bispecific antibody comprising non-mutant (wild-type) CH1 and CL domains and exhibits higher heavy chain (or heavy chain variable region-CH1)-light chain dimerization rates and/or stability, compared to a bispecific antibody comprising non-mutant (wild-type) CH1 and CL domains.

The bispecific antibody comprising mutant CH1 and CL domains may comprise modified CH3 domains inclusive of a first CH3 domain derived from an antibody recognizing a first epitope and a second CH3 domain derived from an antibody recognizing a second epitope, or an Fc region comprising the modified CH3 domain, wherein the first CH3 domain and the second CH3 domain have at least one of the following mutations:

(1) a mutation in which one member of at least one amino acid pair between the CH3 domains is substituted with an amino acid having a positive charge and the other member is substituted with an amino acid having a negative charge;

(2) a mutation in which the members of at least one amino acid pair between the CH3 domains are switched with each other; and

(3) a mutation in which one member of at least one amino acid pair between the CH3 domains is substituted with a large amino acid (e.g., large hydrophobic amino acids such as tryptophan, phenylalanine, etc.) and the other member is substituted with a small amino acid (e.g., small hydrophobic amino acids such as alanine, glycine, valine, etc.).

A bispecific protein or bispecific antibody comprising the mutant CH1 domain, the mutant CL domain, and the mutant Fc region or modified CH3 domain may exhibit an improvement in heterodimeration rate, dimerization rate between a heavy chain (or heavy chain variable region-CH1) and a light chain, both targeting the same epitope, and/or stability, compared to a bispecific protein or antibody comprising a CH1 domain, a CL domain, and an Fc region or CH3 domain none of which are mutant.

The bispecific antibody comprising mutant CH1 and CL domains or the antigen-binding fragment thereof targets the same epitopes as those for a bispecific antibody comprising non-mutant (wild-type) CH1 and CL domains and exhibits higher heavy chain (or heavy chain variable region-CH1)-light chain dimerization rates and/or stability, compared to a bispecific antibody comprising non-mutant (wild-type) CH1 and CL domains.

Another embodiment provides a method for enhancing heterodimerization of a bispecific protein for targeting different targets, the bispecific protein comprising modified CH3 domains or an Fc region comprising the modified CH3 domains, said method comprising one of the following mutation introducing steps:

(1) substituting one of the two amino acids in at least one amino acid pair selected from amino acid pairs forming amino-amino acid bonds between the CH3 domains with an amino acid having a positive charge and the other with an amino acid having a negative charge;

(2) switching the two amino acids in at least one amino acid pair selected from amino acid pairs forming amino-amino acid bonds between a first CH3 domain and a second CH3 domain with each other; and

(3) substituting one of the two amino acids in at least one amino acid pair selected from amino acid pairs forming amino-amino acid bonds between the first CH3 domain and the second CH3 domain with a large hydrophobic amino acid (for example, tryptophan, phenylalanine, etc.) and the other with a small hydrophobic amino acid (for example, alanine, glycine, valine, etc.).

Another embodiment provides a method for constructing a bispecific antibody or an antigen-binding fragment thereof or for enhancing a dimerization rate between a heavy chain (or heavy chain variable region-CH1) and a light chain, both targeting the same epitope, the method comprising the following CH1 and CL domain mutating steps of:

substituting one of the two amino acid residues constituting at least one selected from amino acid pairs forming amino acid-amino acid bonds between a first CH3 domain derived from the heavy chain and a first CL domain of an antibody recognizing a first epitope with an amino acid having a positive charge and the other with an amino acid having a negative charge; and

substituting one of the two amino acid residues constituting at least one selected from amino acid pairs forming amino acid-amino acid bonds between a second CH3 domain derived from the heavy chain and a second CL domain of an antibody recognizing a second epitope with an amino acid having a positive charge and the other with an amino acid having a negative charge.

The method for constructing a bispecific antibody or an antigen-binding fragment thereof or for enhancing a dimerization rate between a heavy chain (or heavy chain variable region-CH1) and a light chain may comprise, in addition to the CH1 and CL domain mutating steps, at least one of the following CH3 domain mutation steps:

(1) substituting one of the two amino acids in at least one selected from amino acid pairs forming amino-amino acid bonds between the first CH3 domain derived from the heavy chain of an antibody recognizing a first epitope and the second CH3 domain derived from the heavy chain of an antibody recognizing a second epitope with an amino acid having a positive charge and the other with an amino acid having a negative charge;

(2) switching the two amino acids in at least one amino acid pair selected from amino acid pairs forming amino-amino acid bonds between the first CH3 domain and the second CH3 domain with each other; and

(3) substituting one of the two amino acids in at least one amino acid pair selected from amino acid pairs forming amino-amino acid bonds between the first CH3 domain and the second CH3 domain with a large hydrophobic amino acid (for example, tryptophan, phenylalanine, etc.) and the other with a small hydrophobic amino acid (for example, alanine, glycine, valine, etc.).

Another embodiment provides a method for constructing a bispecific antibody or an antigen-binding fragment thereof and for enhancing heterodimerization of a bispecific antibody or an antigen-binding fragment thereof for targeting different targets, the method comprising one of the following mutation introducing steps to introduce at least one mutation into at least one selected from amino acid pairs forming amino acid-amino acid bonds between a first CH3 domain and a second CH3 domain.

(1) substituting one of the two amino acids in at least one selected from amino acid pairs forming amino-amino acid bonds between the first CH3 domain derived from the heavy chain of an antibody recognizing a first epitope and the second CH3 domain derived from the heavy chain of an antibody recognizing a second epitope with an amino acid having a positive charge and the other with an amino acid having a negative charge;

(2) switching the two amino acids in at least one amino acid pair selected from amino acid pairs forming amino-amino acid bonds between the first CH3 domain and the second CH3 domain with each other; and

(3) substituting one of the two amino acids in at least one amino acid pair selected from amino acid pairs forming amino-amino acid bonds between the first CH3 domain and the second CH3 domain with a large hydrophobic amino acid (for example, tryptophan, phenylalanine, etc.) and the other with a small hydrophobic amino acid (for example, alanine, glycine, valine, etc.).

The method for constructing a bispecific antibody or an antigen-binding fragment thereof may further comprise the following CH1 and CL domain mutating steps of:

substituting one of the two amino acid residues constituting at least one selected from amino acid pairs forming amino acid-amino acid bonds between a first CH3 domain derived from the heavy chain and a first CL domain of an antibody recognizing a first epitope with an amino acid having a positive charge and the other with an amino acid having a negative charge; and

substituting one of the two amino acid residues constituting at least one selected from amino acid pairs forming amino acid-amino acid bonds between a second CH3 domain derived from the heavy chain and a second CL domain of an antibody recognizing a second epitope with an amino acid having a positive charge and the other with an amino acid having a negative charge.

The method for constructing a bispecific antibody or an antigen-binding fragment thereof can enhance heterodimerization between CH3 domains or Fc regions derived from antibodies recognizing different epitope as well as between CH1 domains (heavy chains) and CL domains (light chains) derived from antibodies recognizing the same epitope.

### Technical Solution

Below, a detailed description will be given of the present invention.

The present invention provides a bispecific protein comprising an Fc constant region and/or an Fab constant region and targeting different targets and a construction method therefor, wherein an amino acid mutation is introduced to the Fc constant regions (CH3 domains) linked (fused) respectively to targeting domains different from each other to increase coupling between the Fc constant regions linked to different targeting domains, thereby increasing a heterodimerization rate between the Fc constant regions linked to different targeting domains and decreasing a homodimerization rate between the Fc constant regions linked to the same targeting domain; and/or

an amino acid mutation is introduced to Fab constant regions linked to targeting domains different from each other to increase coupling between the Fab constant regions linked to different targeting domains, thereby a dimerization rate between the same targeting domains and between the Fab constant regions linked thereto,

whereby a bispecific protein having different targeting domains can be produced at high yield.

Amino acid positions in the antibodies (heavy and light chains), CH1 domains, CL domains, Fc regions, and CH3 domains disclosed in the description are all given as numbered according to the EU numbering system [the EU-index set forth in “Kabat et al., Sequences of Proteins of Immunological Interest, 5th Ed. Public Health Service, National Institutes of Health, Bethesda, Md. (1991)”] and differ from numbers accounting for positions of sequences in the sequencing list.

The antibody may be at least one selected from among all kinds of immunoglobulins originating from mammals or birds. For example, the antibody used in the description may be at least one selected from the group consisting of IgG (e.g., IgG type 1 (IgG1), IgG type 2 (IgG2), IgG type 3 (IgG3), and IgG type 4 (IgG4)), IgA (e.g., IgA type 1 (IgA1) and IgA type 2 (IgA2)), IgD, IgE, and IgM. The antibody may be an immunoglobulin derived from mammals such as primates comprising humans, monkeys, etc. and rodents comprising mice, rats, etc. and may be, for example, a human-derived immunoglobulin. In one embodiment, the antibody may be at least one selected from among human IgG1 (constant region; protein: GenBank Accession No. AAC82527.1, gene: GenBank Accession No. J00228.1), human IgG2 (constant region; protein: GenBank Accession No. AAB59393.1, gene: GenBank Accession No. J00230.1), human IgG3 (constant region; protein: GenBank Accession No. P01860, gene: GenBank Accession No. X03604.1), human IgG4 (constant region; protein: GenBank Accession No. AAB59394.1, gene: GenBank Accession No. K01316.1), human IgA1 (constant region; protein: GenBank Accession No. AAT74070.1, gene: GenBank Accession No. AY647978.1), human IgA2 (constant region; protein: GenBank Accession No. AAB59396.1, gene: GenBank Accession No. J00221.1), human IgD (constant region; protein: GenBank Accession No. AAA52771.1, AAA52770.1), human IgE (constant region; protein: GenBank Accession No. AAB59395.1, gene: GenBank Accession No. J00222.1), and human IgM (constant region; protein: GenBank Accession No. CAB37838.1, gene: GenBank Accession No. X57086.1). In one embodiment, the antibody may be at least one selected from the group consisting of human-derived IgG1, IgG2, IgG3, and IgG4, but is not limited thereto. The 1^{st} CH3 domain and the 2^{nd} CH3 domain, the 1^{st} CH1 domain and the 1^{st} CL domain, and the 2^{nd} CH1 domain and the 2^{nd} CL domain into all of which a mutation is introduced may each be independently selected from among identical or different immunoglobulin types.

As can be seen in FIG. 33a depicting sequence alignment results of the human IgG1 heavy chain constant region (SEQ ID NO: 33) and the human IgA1 heavy chain constant region (SEQ ID NO: 34) and in FIG. 33b depicting sequence alignment results of the kappa constant region (SEQ ID NO: 35) and lambda constant region (SEQ ID NO: 36) of the human immunoglobulin light chain, the heavy chain constant region and light chain constant region exhibit highly conserved amino acid sequences between subtypes.

In addition, immunoglobulin sequences are highly conserved among species and subtypes from which the sequences are derived. For instance, as shown by the sequence alignment results of heavy chain constant regions among human, mouse, and rat in FIG. 33c (CH1 domain sequence alignment) and FIG. 33d (CH3 domain sequence alignment), the amino acid sequences of heavy chain constant region of immunoglobulins are of high interspecies conservation.

In the description, hence, reference is given to the human IgG1 to designate amino acid positions in the CH1 domains and CH3 domains and to the human kappa constant region to designate amino acid positions in the CL domains. The amino acid positions designated with the human IgG1 and the human kappa constant region serving as references allows corresponding amino acid positions in immunoglobulins of other subtypes and immunoglobulins of species other than humans to be explicitly designated through a typical sequence alignment means (e.g., https://blast.ncbi.nlm.nih.gov/Blast.cgi) (see Table 1).

In addition, amino acid positions in the CH1 domain, CL domain, and CH3 domain, provided in the description, are represented according to the EU numbering system, and with respect to the details thereof, reference may be made to “http://www.imgt.org/EVIGTScientificChart/Numberingiflu_IGHGnber.html (heavy chain constant region)”, “http://www.imgt.org/EVIGTScientificChart/Numbering/Hu_IGLCnber.html (light chain lambda region)” and “http://www.imgt.org/EVIGTScientificChart/Numberingiflu_IGKCnber.html (light chain kappa region)”.

Using the human IgG1 as a reference, the EU numbering system numbers:

(1) the CH1 domain (SEQ ID NO: 1) consecutively, with the first amino acid residue (Ala) given position 118 (i.e., 108 amino acid residues of the CH1 domain of SEQ ID NO: 1 correspond respectively to positions 118 to 215 in IgG1); and

(2) the CH3 domain (SEQ ID NO: 15) consecutively, with the first amino acid residue (Lys) given position 340 (i.e., 108 amino acid residues of the CH3 domain of SEQ ID NO: 15 corresponds respectively to positions 340 to 447 in IgG1) (see, http://www.imgt.org/IMGTScientificChart/Numbering/Hu_IGHGnber html).

In the description, amino acid positions in the CH1 and CH3 domains and amino acid kinds corresponding thereto are depicted, with the human IgG1 serving as a reference.

Further, according to the EU numbering,

the CL domain (SEQ ID NO: 10) of the human kappa constant region (protein: GenBank Accession No. AAA58989.1 gene: GenBank Accession No. J00241.1) is numbered consecutively, with the first amino acid residue (Val) given position 110 (i.e., 105 amino acid residues of the CL domain of SEQ ID NO: 10 correspond respectively to positions 110 to 214; see http://www.imgt.org/EVIGTScientificChart/Numbering/Hu_IGKCnber.html); and

the CL domains (SEQ ID NO: 11 (Lambda1), SEQ ID NO: 12 (Lambda2), SEQ ID NO: 13 (Lambda3), and SEQ ID NO: 14 (Lambda1)) of the human lambda constant region are numbered consecutively, with the first amino acid residue (Lys) given position 110 (for the lambda constant region, positions 169, 201, and 203 are omitted from the serial number established; that is, the 103 amino acid residues of the CL domain of SEQ ID NO: 11 or 12 are numbered from position 110 to position 168, from position 170 to position 200, and from position 203 to position 215; see http://www.imgtorg/IMGTScientificChart/Numbering/Hu_IGLCnber html) In the description, amino acid positions in the CL domains and amino acid kinds corresponding thereto are depicted, with the human kappa constant region serving as a reference.

The Fab constant region may comprise one heavy chain constant region (i.e., CH1 domain) selected from the group consisting of heavy chain constant regions of Fab fragments of IgG (IgG1, IgG2, IgG3, and IgG 4), IgA (IgA1 and IgA2), IgD, IgE, and IgM and one light chain constant region (i.e. CL domain) selected from the group consisting of the kappa type and lambda types (e.g., lambda type 1, lambda type 2, lambda type 3, and lambda type 7) of immunoglobulin light chains.

By way of example, the CH1 domains of IgG1, IgG2, IgG3, IgG4, IgA1, IgA2, IgD, IgE, and IgM, which are each available as a heavy chain constant region (CH1 domain) of the Fab fragment may comprise the amino acid sequences of SEQ ID NO: 1 (corresponding to positions 118 to 215 according to EU numbering), SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, respectively. In addition, the CL domains of the kappa type, lambda type 1, lambda type 2, lambda type 3, and lambda type 7 are each available as the light chain constant region (CL domain) and may comprise the amino acid sequences of SEQ ID NO: 10 (corresponding to positions 110 to 214 according to EU numbering), SEQ ID NO: 11, SEQ ID NO: 12, SEQ ID NO: 13, and SEQ ID NO: 14, respectively. In an embodiment, the Fab constant region the CH1 domain (SEQ ID NO: 1) of IgG type 1 and the light chain constant region (CL domain) (SEQ ID NO: 10) of the kappa type. In order to enhance dimerization between molecules targeting the same subject, the amino acid substituted with a negatively charged amino acid or a positively charged amino acid in the CH1 domain may be at least one residue, for example, one, two or more (e.g., two), three or more (e.g., three), or four or more (e.g., four) residues selected from the group consisting of leucine at position 145, lysine at position 147, phenylalanine at position 170, serine at position 183, and valine at position 185 in IgG type 1 (SEQ ID NO: 1) as numbered according to the EU numbering system. In another embodiment, the amino acid substituted with a negatively charged amino acid or a positively charged amino acid in the CH1 domain may be at least one residue selected from the group consisting of amino acids of other IgG subtypes (IgG2, IgG3, and IgG4), IgA1, IgA2, IgD, IgE, and IgM (respectively SEQ ID NOS: 2 to 9) at positions corresponding to leucine at position 145, lysine at position 147, phenylalanine at position 170, serine at position 183, and valine at position 185 on the amino acid sequence of SEQ ID NO: 1.

As used herein, “amino acids at positions corresponding to” can be determined by typical sequence alignment of the amino acid sequence of SEQ ID NO: 1 with target amino acid sequences (i.e., SEQ ID NOS: 2 to 9) without difficulty (hereinafter, the same will be applied).

The amino acid having a positive charge (positively charged amino acid) may be selected from basic amino acids and may be, for example, lysine or arginine. When an amino acid having a positive charge is introduced into the CH1 domain, at least one residue, for example, one, two or more (e.g., two), three or more (e.g., three), or four or more (e.g., four) residues selected from the group consisting of leucine at position 145, lysine at position 147, phenylalanine at position 170, proline at position 171, serine at position 183, and valine at position 185 on the amino acid sequence of SEQ ID NO: 1, and amino acids at positions corresponding thereto on the amino acid sequences of SEQ ID NOS: 2 to 9 may each be independently substituted by a basic amino acid, for example, lysine or arginine.

For example, in order to introduce an amino acid having a positive charge thereinto, the CH1 domain may comprise at least one, two or more (e.g., two), three or more (e.g., three), or four or more (e.g., four) of the following mutations therein (on the amino acid sequence of SEQ ID NO: 1; and also applied to amino acid residues at positions corresponding thereto on the amino acid sequences of SEQ ID NOS: 2 to 9):

substitution of leucine at position 145 with lysine or arginine (e.g., lysine);

substitution of serine at position 183 with lysine or arginine (e.g., lysine);

substitution of lysine at position 147 with arginine;

substitution of phenylalanine at position 170 with lysine or arginine (e.g., lysine);

substitution of proline at position 171 with lysine or arginine (e.g., lysine); and

substitution of valine at position 185 with lysine or arginine (e.g., arginine).

The amino acid having a negative charge may be selected from among acidic amino acid residues and may be, for example, aspartic acid or glutamic acid. Hence, when an amino acid having a negative charge is introduced into the CH1 domain, at least one, for example, one, two or more (e.g., two), three or more (e.g., three), or four or more (e.g., four) residues selected from the group consisting of leucine at position 145, lysine at position 147, phenylalanine at position 170, proline at position 171, serine at position 183, and valine at position 185 on the amino acid sequence of SEQ ID NO: 1, and amino acids at positions corresponding thereto on the amino acid sequences of SEQ ID NOS: 2 to 9 may each be independently substituted by an acidic amino acid, for example, aspartic acid or glutamic acid. For example, in order to introduce an amino acid having a positive charge thereinto, the CH1 domain may comprise at least one, two or more (e.g., two), three or more (e.g., three), or four or more (e.g., four) of the following mutations (on the amino acid sequence of SEQ ID NO: 1; and also applied to amino acid residues at positions corresponding thereto on the amino acid sequences of SEQ ID NOS: 2 to 9):

substitution of leucine at position 145 with aspartic acid or glutamic acid (e.g., glutamic acid);

substitution of lysine at position 147 with aspartic acid or glutamic acid (e.g., aspartic acid);

substitution of serine at position 183 with aspartic acid or glutamic acid (e.g., glutamic acid);

substitution of valine at position 185 with aspartic acid or glutamic acid (e.g., aspartic acid);

substitution of phenylalanine at position 170 with aspartic acid or glutamic acid (e.g., aspartic acid); and

substitution of proline at position 171 with aspartic acid or glutamic acid (e.g., aspartic acid).

In order to enhance dimerization between molecules targeting the same subject, the amino acid substituted with a negatively charged amino acid or a positively charged amino acid in the light chain constant region (CL domain) may be at least one residue, for example, one, two or more (e.g., two), three or more (e.g., three), or four or more (e.g., four) residues selected from the group consisting of serine at position 131, valine at position 133, leucine at position 135, serine at position 162, and threonine at position 180 in in the kappa type (SEQ ID NO: 10). In another embodiment, the amino acid substituted with a negatively charged amino acid or a positively charged amino acid in the CL domain may be at least one residue selected from the group consisting of amino acids of the CL domains of lambda types (lambda type 1, lambda type 2, lambda type 3, and lambda type 7) (respectively SEQ ID NOS: 11 to 14) at positions corresponding to serine at position 131, valine at position 133, leucine at position 135, serine at position 162, and threonine at position 180 on the amino acid sequence of SEQ ID NO: 10.

The amino acid having a positive charge (positively charged amino acid) may be selected from basic amino acids and may be, for example, lysine or arginine Hence, when an amino acid having a positive charge is introduced into the CL domain, at least one residue, for example, one, two or more (e.g., two), three or more (e.g., three), or four or more (e.g., four) residues selected from the group consisting of serine at position 131, valine at position 133, leucine at position 135, serine at position 162, and threonine at position 180 on the amino acid sequence of SEQ ID NO: 10, and amino acids at positions corresponding thereto on the amino acid sequences of SEQ ID NOS: 11 to 14 may each be independently substituted by a basic amino acid, for example, lysine or arginine.

By way of example, in order to introduce an amino acid having a positive charge thereinto, the CL domain may comprise at least one, for example, one, two or more (e.g., two), three or more (e.g., three), or four or more (e.g., four) of the following mutations (for the amino acid sequence of SEQ ID NO: 10; and also applied to the amino acids at positions corresponding thereto on the amino acid sequences of SEQ ID NOS: 11 to 14):

substitution of serine at position 131 with lysine or arginine (e.g., lysine);

substitution of valine at position 133 with lysine or arginine (e.g., lysine);

substitution of leucine at position 135 with lysine or arginine (e.g., arginine);

substitution of serine at position 162 with lysine or arginine (e.g., lysine); and

substitution of threonine at position 180 with lysine or arginine (e.g., arginine).

In order to further enhance homodimerization, the mutant CH1 domain and/or the mutant CL domain may comprise two or more mutations simultaneously.

For example, lysine at position 147 and valine at position 185 in the CH1 domain may be substituted by an amino acid having a positive or negative charge. By way of example, lysine at position 147 and valine at position 185 in one of the first and the second CH1 domain may be substituted by an amino acid having a positive charge (e.g., lysine or arginine) while lysine at position 147 and valine at position 185 in the other CH1 domain may be substituted with an amino acid having a negative charge (glutamic acid or aspartic acid). In addition, leucine at position 135 and threonine at position 180 in the CL domain may be substituted with an amino acid having a positive or negative charge. By way of example, leucine at position 135 and threonine at position 180 in one of the first and the second CH1 domain may be substituted with an amino acid having a positive charge (e.g., lysine or arginine) while leucine at position 135 and threonine at position 180 in the other CL domain may be substituted with an amino acid having a negative charge (glutamic acid or aspartic acid).

In another embodiment, phenylalanine at position 170 and proline at position 171 in the CH1 domain may be substituted with an amino acid having a positive or negative charge. By way of example, phenylalanine at position 170 and proline at position 171 in one of the first CH1 domain and the second CH1 domain may be substituted with an amino acid having a positive charge (e.g., lysine or arginine) while phenylalanine at position 170 and proline at position 171 in the other CH1 domain may be substituted with an amino acid having a negative charge (glutamic acid or aspartic acid). In addition, substitution with an amino acid having a positive or negative charge may be carried out for leucine at position 135 and serine at position 162 in the CL domain By way of example, leucine at position 135 and serine at position 162 in one of the first CL domain and the second CL domain may be substituted with an amino acid having a positive charge (e.g., lysine or arginine) while leucine at position 135 and serine at position 162 in the other CL domain may be substituted with an amino acid having a negative charge (e.g., glutamic acid or aspartic acid).

The amino acid having a negative charge may be selected from among acidic amino acids and may be, for example, aspartic acid or glutamic acid. Hence, when an amino acid having a negative charge is introduced into the CL domain, at least one residue, for example, one, two or more (e.g., two), three or more (e.g., three), or four or more (e.g., four) residues selected from the group consisting of serine at position 131, valine at position 133, leucine at position 135, serine at position 162, and threonine at position 180 on the amino acid sequence of SEQ ID NO: 10, and amino acids at positions corresponding thereto on the amino acid sequences of SEQ ID NOS: 11 to 14 may each be independently substituted by a basic amino acid, for example, aspartic acid or glutamic acid. By way of example, in order to introduce an amino acid having a positive charge thereinto, the CL domain may comprise at least one, for example, one, two or more (e.g., two), three or more (e.g., three), or four or more (e.g., four) of the following mutations (on the amino acid sequence of SEQ ID NO: 10; and also applied to amino acid residues at positions corresponding thereto on the amino acid sequences of SEQ ID NOS: 11 to 14):

substitution of serine at position 131 with aspartic acid or glutamic acid (e.g., glutamic acid);

substitution of valine at position 133 with aspartic acid or glutamic acid (e.g., glutamic acid);

substitution of leucine at position 135 with aspartic acid or glutamic acid (e.g., aspartic acid);

substitution of serine at position 162 with aspartic acid or glutamic acid (e.g., aspartic acid); and

substitution of threonine at position 180 with aspartic acid or glutamic acid (e.g., aspartic acid).

In one embodiment, a set of two amino acids forming an amino acid pair between the CH1 domain and the CL domain that are to be substituted with a pair of amino acids having opposite charges may be at least one, for example, one, two or more (e.g. two), three or more (e.g., three), or four or more (e.g., four) pairs selected from the group consisting of a pair of leucine at position 145 in the CH1 domain and serine at position 131 in the CL domain, a pair of leucine at position 145 in the CH1 domain and valine at position 133 in the CL domain, a pair of lysine at position 147 in the CH1 domain and threonine at position 180 in the CL domain, a pair of serine at position 183 in the CH1 domain and valine at position 133 in the CL domain, a pair of valine at position 185 in the CH1 domain and leucine at position 135 in the CL domain, a pair of phenylalanine at position 170 in the CH1 domain and leucine at position 135 in the CL domain, and a pair of proline at position 171 in the CH1 domain and serine at position 162 in the CL domain, as numbered on the basis of the amino acid sequences of SEQ ID NO: 1 (CH1 domain) and SEQ ID NO: 10 (CL domain) For example, the amino acid pair between the CH1 and the CL domain to which the mutation is introduced may be one or two or more (e.g., two) pairs selected from the group consisting of a pair of leucine at position 145 in the CH1 domain and serine at position 131 in the CL domain, a pair of leucine at position 145 in the CH1 domain and valine at position 133 in the CL domain, a pair of lysine at position 147 in the CH1 domain and threonine at position 180 in the CL domain, a pair of serine at position 183 in the CH1 domain and valine at position 133 in the CL domain, and a pair of valine at position 185 in the CH1 domain and leucine at position 135 in the CL domain, as numbered on the basis of the amino acid sequences of SEQ ID NO: 1 (CH1 domain) and SEQ ID NO: 10 (CL domain).

The amino acid pairs to which the mutations are introduced between the first CH1 domain and the first CL domain may be the same as or different from those between the second CH1 domain and the second CL domain.

In one embodiment, an amino acid having a positive charge is introduced to the first CH1 domain (with the introduction of an amino acid having a negative charge to the first CL domain) while an amino acid having a negative charge is introduced to the second CH1 domain (with the introduction of an amino acid having a positive charge to the second CL domain).

In order to enhance heterodimerization, at least one, for example, one, two, or three of the following mutations may be introduced to the Fc region of a heavy chain, in detail, the CH3 domain in the Fc region:

(1) mutation in which one amino acid residue of at least one amino acid pair (at least one of the two amino acid residues is not hydrophobic) between CH3 domains is substituted with an amino acid having a positive charge while the other residue is substituted with an amino acid having a negative charge (hereinafter referred to as “electrostatic interaction-induced mutation”);

(2) mutation in which amino acid residues in at least one amino acid pair between the CH3 domains are exchanged with each other (hereinafter referred to as “swapping mutation”); and

(3) mutation in which one amino acid residue in at least one amino acid pair between the CH3 domains is substituted with a large amino acid (e.g., a large hydrophobic amino acid such as tryptophan, phenylalanine, etc.) while the other amino acid residue is substituted with a small amino acid (e.g., a small hydrophobic amino acid such as alanine, glycine, valine, etc.) (hereinafter referred to as “size mutation”).

As such, the CH3 domain to which the mutations are introduced may be selected from the group consisting of the CH3 domain of human IgG1(SEQ ID NO: 15; corresponding to positions 340 to 447 according to the EU numbering), the CH3 domain of human IgG2 (SEQ ID NO: 16), the CH3 domain of human IgG3(SEQ ID NO: 17), the CH3 domain of human IgG4 (SEQ ID NO: 18), the CH3 domain of human IgA1 (SEQ ID NO: 19), the CH3 domain of human IgA2 (SEQ ID NO: 20), the CH3 domain of human IgD (SEQ ID NO: 21), the CH3 domain of human IgE (SEQ ID NO: 22), and the CH3 domain of human IgM (SEQ ID NO: 23).

The first 1 CH3 domain and the second CH3 domain to which the mutations are introduced may be derived from the same or different immunoglobulin types which may each be independently selected from the group consisting of IgG1, IgG2, IgG3, IgG4, IgA1, IgA2, IgD, IgE, and IgM. In one embodiment, the first CH3 domain and the second CH3 domain may both be the CH3 domain of human IgG1 having the amino acid sequence of SEQ ID NO: 15, but is not limited thereto.

The following amino acid pairs between the first CH3 domain and the second CH3 domain are based on the CH3 domain of human IgG1 having the amino acid of SEQ ID NO: 15, and the basis is true of the amino acid pairs corresponding thereto between CH3 domains of IgG2, IgG3, IgG4, IgA1, IgA2, IgD, IgE, and IgM (respectively, SEQ ID NOS: 16 to 23).

The amino acid pair between the first and second CH3 domains to which one of the mutations is introduced may be at least one, for example, one, two or more (e.g., two), three or more (e.g., three), or four or more (e.g., four) amino acid pairs selected from the group consisting of the amino acid pairs between CH3 domains and amino acid pairs at positions corresponding thereto between CH3 domains of IgG2, IgG3, IgG4, IgA1, IgA2, IgE, and IgM (respectively, SEQ ID NOS: 16 to 23), listed in Table 1, below.

TABLE 1

| Amino | | | | | | | | | | |
|---|---|---|---|---|---|---|---|---|---|---|
| No. | IgG (1-4) | | IgA1 | | IgA2 | | IgE | | IgM | |
| acid pair | Chain A | Chain B | Chain A | Chain B | Chain A | Chain B | Chain A | Chain B | Chain A | Chain B |
| 1 | Q347 | K360 | E347 | L360 | E347 | L360 | E444 | R457 | Q347 | K360 |
| 2 | Y349 | S354 | H349 | P354 | H349 | P354 | Y446 | P451 | Y349 | S354 |
| 3 | Y349 | E357 | H349 | E357 | H349 | E357 | Y446 | P454 | Y349 | E357 |
| 4 | Y349 | K360 | H349 | L360 | H349 | L360 | Y446 | R457 | Y349 | K360 |
| 5 | L351 | L351 | L351 | L351 | L351 | L351 | F448 | F448 | L351 | L351 |
| 6 | P352 | P352 | P352 | P352 | P352 | P352 | A449 | A449 | P352 | P352 |
| 7 | S354 | Y349 | P354 | H349 | P354 | H349 | P451 | Y446 | S354 | Y349 |
| 8 | D356 | K439 | E356 | K439 | E356 | K439 | W453 | R539 | D356 | K439 |
| 9 | E357 | Y349 | E357 | H349 | E357 | H349 | P454 | Y446 | E357 | Y349 |
| 10 | E357 | K370 | E357 | R370 | E357 | R370 | P454 | Q467 | E357 | K370 |
| 11 | K360 | Q347 | L360 | E347 | L360 | E347 | R457 | E444 | K360 | Q347 |
| 12 | K360 | Y349 | L360 | H349 | L360 | H349 | R457 | Y446 | K360 | Y349 |
| 13 | S364 | L368 | T364 | L368 | T364 | L368 | T461 | L465 | S364 | L368 |
| 14 | S364 | K370 | T364 | R370 | T364 | R370 | T461 | Q467 | S364 | K370 |
| 15 | T366 | T366 | T366 | T366 | T366 | T366 | A463 | A463 | T366 | T366 |
| 16 | T366 | Y407 | T366 | T407 | T366 | T407 | A463 | F506 | T366 | Y407 |
| 17 | L368 | S364 | L368 | T364 | L368 | T364 | L465 | T461 | L368 | S364 |
| 18 | L368 | K409 | L368 | I409 | L368 | I409 | L465 | R508 | L368 | K409 |
| 19 | K370 | E357 | R370 | E357 | R370 | E357 | Q467 | P454 | K370 | E357 |
| 20 | K370 | S364 | R370 | T364 | R370 | T364 | Q467 | T461 | K370 | S364 |
| 21 | K370 | T411 | R370 | R411 | R370 | R411 | Q467 | E510 | K370 | T411 |
| 22 | N390 | S400 | L390 | S400 | L390 | S400 | R489 | K499 | N390 | S400 |
| 23 | K392 | L398 | L392 | Q398 | L392 | Q398 | S491 | K497 | K392 | L398 |
| 24 | T394 | T394 | W394 | W394 | W394 | W394 | T493 | T493 | T394 | T394 |
| 25 | T394 | V397 | W394 | R397 | W394 | R397 | T493 | R496 | T394 | V397 |
| 26 | P395 | P395 | A395 | A395 | A395 | A395 | Q494 | Q494 | P395 | P395 |
| 27 | P395 | V397 | A395 | R397 | A395 | R397 | Q494 | R496 | P395 | V397 |
| 28 | V397 | T394 | R397 | W394 | R397 | W394 | R496 | T493 | V397 | T394 |
| 29 | V397 | P395 | R397 | A395 | R397 | A395 | R496 | Q494 | V397 | P395 |
| 30 | L398 | K392 | Q398 | L392 | Q398 | L392 | K497 | S491 | L398 | K392 |
| 31 | S400 | N390 | S400 | K390 | S400 | K390 | K499 | R489 | S400 | N390 |
| 32 | F405 | K409 | A405 | I409 | A405 | I409 | F504 | R508 | F405 | K409 |
| 33 | Y407 | T366 | T407 | T366 | T407 | T366 | F506 | A463 | Y407 | T366 |
| 34 | Y407 | Y407 | T407 | T407 | T407 | T407 | F506 | F506 | Y407 | Y407 |
| 35 | Y407 | K409 | T407 | I409 | T407 | I409 | F506 | R508 | Y407 | K409 |
| 36 | K409 | L368 | I409 | L368 | I409 | L368 | R508 | L465 | K409 | L368 |
| 37 | K409 | F405 | I409 | A405 | I409 | A405 | R508 | F504 | K409 | F405 |
| 38 | K409 | Y407 | I409 | T407 | I409 | T407 | R508 | F506 | K409 | Y407 |
| 39 | T411 | K370 | R411 | R370 | R411 | R370 | E510 | Q467 | T411 | K370 |
| 40 | K439 | D356 | K439 | K439 | K439 | K439 | R539 | W453 | K439 | D356 |

(Chain A: first CH3 domain;
Chain B: second CH3 domain)

In greater detail, the amino acid pair between the first CH3 domain and the second CH3 domain to which at least one of the following mutations: (1) electrostatic interaction-induced mutation (represented as charge (J) in Table 2 and FIG. 2); (2) swapping mutation (represented as swap (O) in Table 2 and FIG. 2); and (3) size mutation (represented as size (B) in Table 2 and FIG. 2) may be at least one, for example, one, two or more (e.g., two), three or more (e.g., three), or four or more (e.g., four) amino acid pairs selected from the amino acid pairs between CH3 domains of IgG1 (SEQ ID NO: 15) and amino acid pairs at positions corresponding thereto between CH3 domains of IgG2, IgG3 IgG4 IgA1 IgA2 IgD, IgE, and IgM (respectively, SEQ ID NOS: 16 to 23), suggested in Table 2 and FIG. 2:

TABLE 2

| Amino acid pair | Charge (J) | | Swap (O) | | Size (B) | |
|---|---|---|---|---|---|---|
| No. | Chain A | Chain B | Chain A | Chain B | Chain A | Chain B |
| 1 | Q347 | K360 | Q347 | K360 | Q347 | K360 |
| 2 | Y349 | S354 | | | Y349 | S354 |
| 3 | Y349 | E357 | | | Y349 | E357 |
| 4 | Y349 | K360 | | | Y349 | K360 |
| 5 | L351 | L351 | | | L351 | L351 |
| 6 | P352 | P352 | | | P352 | P352 |
| 7 | S354 | Y349 | S354 | Y349 | S354 | Y349 |
| 8 | D356 | K439 | | | D356 | K439 |
| 9 | E357 | Y349 | E357 | Y349 | E357 | Y349 |
| 10 | E357 | K370 | E357 | K370 | E357 | K370 |
| 11 | K360 | Q347 | | | K360 | Q347 |
| 12 | K360 | Y349 | K360 | Y349 | K360 | Y349 |
| 13 | S364 | L368 | S364 | L368 | S364 | L368 |
| 14 | S364 | K370 | S364 | K370 | S364 | K370 |
| 15 | T366 | T366 | | | T366 | T366 |
| 16 | T366 | Y407 | | | T366 | Y407 |
| 17 | L368 | S364 | | | L368 | S364 |
| 18 | L368 | K409 | L368 | K409 | L368 | K409 |
| 19 | K370 | E357 | | | K370 | E357 |
| 20 | K370 | S364 | | | K370 | S364 |
| 21 | K370 | T411 | | | K370 | T411 |
| 22 | N390 | S400 | N390 | S400 | N390 | S400 |
| 23 | K392 | L398 | | | K392 | L398 |
| 24 | T394 | T394 | | | T394 | T394 |
| 25 | T394 | V397 | T394 | V397 | T394 | V397 |
| 26 | P395 | P395 | | | P395 | P395 |
| 27 | P395 | V397 | | | P395 | V397 |
| 28 | V397 | T394 | | | V397 | T394 |
| 29 | V397 | P395 | | | V397 | P395 |
| 30 | L398 | K392 | L398 | K392 | L398 | K392 |
| 31 | S400 | N390 | | | S400 | N390 |
| 32 | F405 | K409 | F405 | K409 | F405 | K409 |
| 33 | Y407 | T366 | Y407 | T366 | Y407 | T366 |
| 34 | Y407 | Y407 | | | Y407 | Y407 |
| 35 | Y407 | K409 | | | Y407 | K409 |
| 36 | K409 | L368 | | | K409 | L368 |
| 37 | K409 | F405 | | | K409 | F405 |
| 38 | K409 | Y407 | | | K409 | Y407 |
| 39 | T411 | K370 | T411 | K370 | T411 | K370 |
| 40 | | | | | K439 | D356 |

(Chain A: first CH3 domain;
Chain B: second CH3 domain;
the mutations listed in Table 2 are given to positions based on IgG1, but are applied to corresponding positions in the CH3 domains of IgG2, IgG3, IgG4, IgA1, IgA2, IgD, IgE, and IgM).

As used herein, for example, “Q347” means glutamine at position 347 on the amino acid sequence of SEQ ID NO: 15 in the CH3 domain of human IgG1, and such notation is true of amino acid residues at positions corresponding thereto in CH3 domains of IgG2, IgG3 IgG4, IgA1, IgA2, IgD, IgE, and IgM (respectively, SEQ ID NOS: 16 to 23) (hereinafter, the same definition is applied).

Hereinafter, amino acid pairs between CH3 domains to which the mutations are introduced are numbered on the basis of the amino acid sequence of SEQ ID NO: 15 and unless otherwise described, the numbering is construed to be applied to amino acids at denoted positions in IgG1 as well as at positions corresponding thereto in CH3 domains of other type immunoglobulins (IgG2, IgG3, IgG4, IgA1, IgA2, IgD, IgE, and IgM).

The electrostatic interaction-induced mutation is intended to substitute a positively charged amino acid for one amino acid residue of at least one amino acid pair between Fc regions or CH3 domains (at least one residue of the paired two amino acids is not hydrophobic) and a negatively charged amino acid for the other amino acid residue to introduce electrostatic interaction to a hydrophobic interaction-lacking site, thereby contributing to an increase of electrostatic interaction-induced binding force.

The hydrophobic amino acid may be selected from the group consisting of glycine, alanine, valine, leucine, isoleucine, methionine, proline, phenylalanine, and tryptophan.

The amino acid having a negative charge may be selected from among acidic amino acids and may be, for example, aspartic acid or glutamic acid. The amino acid having a positive charge may be selected from among basic amino acid and may be, for example, lysine or arginine.

The amino acid pair between the first CH3 domain and the second CH3 domain to which an electrostatic interaction-introduced mutation is applicable may be at least one, for example, one, two or more (e.g., two), three or more (e.g., three), or four or more (e.g., four) selected from among amino acid pair numbers 1 to 39 in Table 2 and may be at least one, for example, one, two or more (e.g., two), three or more (e.g., three), or four or more (e.g., four) selected from the group consisting of, for example, a pair of serine at position 364 and leucine at position 368, a pair of threonine at position 394 and threonine at position 394, a pair of glutamic acid at position 357 and lysine at position 370, a pair of glutamic acid at position 357 and tyrosine at position 349, a pair of threonine at position 366 and tyrosine at position 407, and a pair of threonine at position 394 and valine at position 397.

That is, the electrostatic interaction-introduced mutation in the CH3 domain may comprise substitution of an amino acid having a positive charge for one amino acid residue of each of the amino acid pairs, for example, one, two or more (e.g., two), three or more (e.g., three), or four or more (e.g., four) selected from among the amino acid pair number 1 to 39 in Table 2; and an amino acid having a negative charge for the other amino acid residue. By way of example, in each of the amino acid pairs, for example, one or more (e.g., one), two or more (e.g., two), three or more (e.g., three), or four or more (e.g., four) amino acid pairs selected from the group consisting of a pair of serine at position 364 and leucine at position 368, a pair of threonine at position 394 and threonine at position 394, a pair of glutamic acid at position 357 and lysine at position 370, a pair of glutamic acid at position 357 and tyrosine at position 349, a pair of threonine at position 366 and tyrosine at position 407, and a pair of threonine at position 394 and valine at position 397, one amino acid residue is substituted with an amino acid having a positive charge while the other amino acid residue is substituted with an amino acid having a negative charge.

For instance, the electrostatic interaction-introduced mutation in CH3 domains may comprise at least one, for example, one, two or more (e.g., two), three or more (e.g., three), or four or more (e.g., four) of the following mutations:

substitution of serine at position 364 with an amino acid having a positive charge and leucine at position 368 with an amino acid having a negative charge;

substitution of threonine at position 394 with an amino acid having a positive charge and threonine at position 394 with an amino acid having a negative charge;

substitution of glutamic acid at position 357 with an amino acid having a positive charge and lysine at position 370 with an amino acid having a negative charge;

substitution of glutamic acid at position 357 with an amino acid having a positive charge and tyrosine at position 349 with an amino acid having a negative charge;

substitution of threonine at position 366 with an amino acid having a positive charge and tyrosine at position 407 with an amino acid having a negative charge;

substitution of threonine at position 394 with an amino acid having a positive charge and valine at position 397 with an amino acid having a negative charge; and

substitution of tyrosine at position 349 with an amino acid having a positive charge and glutamic acid at position 357 with an amino acid having a negative charge.

Such an electrostatic interaction introduction in CH3 domains may achieve a heterodimerization rate of 60% or higher, 65% or higher, 70% or higher, 73% or higher, 75% or higher, 78% or higher, 80% or higher, 85% or higher, 90% or higher, 95% or higher, or 100%.

The swapping mutation means a mutation in which two amino acid residues constituting an amino acid pair are exchanged (swapped) with each other.

As illustrated in Table 2 and FIG. 2, the amino acid pair between the first CH3 domain and the second CH3 domain to which the swapping mutation is applicable may be at least one, for example, one, two or more (e.g., two), three or more (e.g., three), or four or more (e.g., four) selected from the group consisting of a pair of glutamine at position 347 and lysine at position 360, a pair of glutamic acid at position 357 and tyrosine at position 349, a pair of serine at position 354 and tyrosine at position 349, a pair of glutamic acid at position 357 and lysine at position 370, a pair of lysine at position 360 and tyrosine at position 349, a pair of serine at position 364 and leucine at position 368, a pair of serine at position 364 and lysine at position 370, a pair of leucine at position 368 and lysine at position 409, a pair of asparagine at position 390 and serine at position 400, a pair of threonine at position 394 and valine at position 397, a pair of leucine at position 398 and lysine at position 392, a pair of phenylalanine at position 405 and lysine at position 409, a pair of tyrosine at position 407 and threonine at position 366, and a pair of threonine at position 411 and lysine at position 370 and particularly from the group consisting of a pair of serine at position 364 and lysine at position 370, a pair of tyrosine at position 407 and threonine at position 366, a pair of glutamic acid at position 357 and lysine at position 370, a pair of phenylalanine at position 405 and lysine at position 409, and a pair of glutamic acid at position 357 and tyrosine at position 349.

That is, the swapping mutation in CH3 domains may comprise substitution in which exchange (swapping) is made between two paired amino acid residues in each of at least one, for example, one, two or more (e.g., two), three or more (e.g., three), or four or more (e.g., four) amino acid pairs selected from the group consisting of a pair of glutamine at position 347 and lysine at position 360, a pair of glutamic acid at position 357 and tyrosine at position 349, a pair of serine at position 354 and tyrosine at position 349, a pair of glutamic acid at position 357 and lysine at position 370, a pair of lysine at position 360 and tyrosine at position 349, a pair of serine at position 364 and leucine at position 368, a pair of serine at position 364 and lysine at position 370, a pair of leucine at position 368 and lysine at position 409, a pair of asparagine at position 390 and serine at position 400, a pair of threonine at position 394 and valine at position 397, a pair of leucine at position 398 and lysine at position 392, a pair of phenylalanine at position 405 and lysine at position 409, a pair of tyrosine at position 407 and threonine at position 366, and a pair of threonine at position 411 and lysine at position 370; and for example, from the group consisting of a pair of serine at position 364 and lysine at position 370, a pair of tyrosine at position 407 and threonine at position 366, a pair of glutamic acid at position 357 and lysine at position 370, a pair of phenylalanine at position 405 and lysine at position 409, and a pair of glutamic acid at position 357 and tyrosine at position 349.

For example, the swapping mutation in CH3 domains may comprise at least one, for example, one, two or more (e.g., two), three or more (e.g., three), or four or more (e.g., four) of the following mutations:

substitution of serine at position 364 with lysine and lysine at position 370 with serine;

substitution of phenylalanine at position 405 with lysine and lysine at position 409 with phenylalanine;

substitution of tyrosine at position 407 with threonine and threonine at position 366 with tyrosine;

substitution of glutamic acid at position 357 with lysine and lysine at position 370 with glutamic acid; and

substitution of glutamic acid at position 357 with tyrosine and tyrosine at position 349 with serine.

Such a swapping mutation in CH3 domains may achieve a heterodimerization rate of 60% or higher, 65% or higher, 70% or higher, 73% or higher, 75% or higher, 78% or higher, 80% or higher, 85% or higher, 90% or higher, 95% or higher, or 100%.

The size mutation means a mutation in which, of the two paired amino acids in at least one amino acid pair between CH3 domains, one residue is substituted with a large hydrophobic amino acid (e.g., tryptophan, phenylalanine, etc.) and the other is substituted with a small hydrophobic amino acid (e.g., alanine, glycine, valine, etc.) so that the large amino acid is fitted to the space secured by the small amino acid, thereby contributing to heterodimerization.

The large amino acid may comprise a cyclic residue and may be selected from the group consisting of tryptophan and phenylalanine, and particularly tryptophan. The small amino acid may be selected from the group consisting of alanine, glycine, and valine and may be, for example, alanine.

The amino acid pair between the first CH3 domain and the second CH3 domain to which the size mutation is applicable may be at least one, for example, one, two or more (e.g., two), three or more (e.g., three), or four or more (e.g., four) selected from among amino acid pair numbers 1 to 40 in Table 2 and may be, for example, a pair of lysine at position 409 and tyrosine at position 407, a pair of lysine at position 409 and phenylalanine at position 405, or a combination thereof.

That is, the size mutation may comprise substitution in which, of two paired amino acid residues in each of at least one, for example, one, two or more (e.g., two), three or more (e.g., three), or four or more (e.g., four) amino acid pairs selected from among amino acid pair numbers 1 to 40 in Table 2, one is substituted with a large hydrophobic amino acid (e.g., tryptophan, phenylalanine, etc.), for example, tryptophan while the other is substituted with a small hydrophobic amino acid (e.g., alanine, glycine, valine, etc.), for example, alanine. By way of example, in a pair of lysine at position 409 and tyrosine at position 407 or a pair of lysine at position 409 and phenylalanine at position 405, or in each of them, one residue may be substituted with a large hydrophobic amino acid, for example, phenylalanine or tryptophan while the other residue may be substituted with a small hydrophobic amino acid, for example, alanine, glycine, or valine.

For example, the size mutation in CH3 domains may comprise at least one of the following mutations:

substitution of lysine at position 409 with tryptophan and tyrosine at position 407 with alanine; and

substitution of lysine at position 409 with tryptophan and phenylalanine at position 405 with alanine.

The modified CH3 domains may comprise at least one, for example, one or two of the three mutations described above, that is, electrostatic interaction-introduced mutation, swapping mutation, and size mutation.

In order to exert the most advantageous effect on dimerization, the modified CH3 domains may comprise at least one, for example, one, two, or three mutations selected from the group consisting of substitution of one residue in a pair of serine at position 364 and leucine at position 368 with an amino acid having a positive charge and the other with an amino acid having a negative charge (electrostatic interaction-introduced mutation), exchange of the two residues of a pair of serine at position 364 and lysine at position 370 with each other (swapping mutation), and exchange of the two residues of a pair of phenylalanine at position 405 and lysine at position 409 with each other (swapping mutation).

For example, the modified CH3 domains may comprise one, two, or three of the following mutations:

(a) substitution of serine at position 364 with an amino acid having a positive charge and leucine at position 368 with an amino acid having a negative charge;

(b) substitution of serine at position 364 with lysine and lysine at position 370 with serine; and

(c) substitution of phenylalanine at position 405 with lysine and lysine at position 409 with phenylalanine.

In order to decrease monomerization rates, but increase dimerization rates, an additional amino acid modification may be introduced after the three selected mutations ((a)-(c)). As such, the amino acid to which the additional amino acid mutation can be introduced may be serine at position 364, phenylalanine at position 405, and/or lysine at position 409. For example, in a pair of serine at position 364 and leucine at position 368 which is to undergo an electrostatic interaction-induced mutation, when leucine at position 368 may be substituted with an amino acid having a negative charge (aspartic acid or glutamic acid, e.g., aspartic acid), serine at position 364 may be substituted with an amino acid having a positive charge (lysine or arginine) (S364K or S364R; electrostatic interaction-introduced mutation) or with asparagine (S364N). In addition, in a pair of lysine at position 370 and serine at position 364, which is to undergo a swapping mutation, lysine at position 370 may be substituted with serine and serine at position 364 may be substituted with lysine (S364K; swapping mutation) or with arginine or asparagine (S364R or S364N).

Further, in a pair of lysine at position 409 and phenylalanine at position 405, which is to undergo swapping mutation, lysine at position 409 may be substituted with phenylalanine (for swapping mutation) or with tryptophan and phenylalanine at position 405 may be substituted with lysine (F405K; for swapping mutation) or with arginine, glutamine, or asparagine (F405R, F405Q, or F405N).

In one embodiment, the modified CH3 domains may comprise at least one of the following mutations:

substitution of serine at position 364 with lysine and leucine at position 368 with aspartic acid;

substitution of serine at position 364 with lysine and lysine at position 370 with serine;

substitution of phenylalanine at position 405 with lysine and lysine at position 409 with phenylalanine;

substitution of phenylalanine at position 405 with arginine and lysine at position 409 with phenylalanine;

substitution of phenylalanine at position 405 with lysine and lysine at position 409 with tryptophan; and

substitution of phenylalanine at position 405 with arginine and lysine at position 409 with tryptophan.

In one embodiment, the modified CH3 domains may comprise at least one of the following double mutations:

substitution of serine at position 364 in the first CH3 domain with lysine and leucine at position 368 in the second CH3 domain with aspartic acid, and substitution of lysine at position 409 in the first CH3 domain with phenylalanine and phenylalanine at position 405 in the second CH3 domain with lysine;

substitution of serine at position 364 in the first CH3 domain with lysine and leucine at position 368 in the second CH3 domain with aspartic acid, and substitution of lysine at position 409 in the first CH3 domain with phenylalanine and phenylalanine at position 405 in the second CH3 domain with arginine;

substitution of serine at position 364 in the first CH3 domain with lysine and lysine at position 370 in the second CH3 domain with serine, and substitution of lysine at position 409 in the first CH3 domain with phenylalanine and phenylalanine at position 405 in the second CH3 domain with lysine;

substitution of serine at position 364 in the first CH3 domain with lysine and lysine at position 370 in the second CH3 domain with serine, and substitution of lysine at position 409 in the first CH3 domain with phenylalanine and phenylalanine at position 405 in the second CH3 domain with arginine;

substitution of serine at position 364 in the first CH3 domain with lysine and leucine at position 368 in the second CH3 domain with aspartic acid, and substitution of lysine at position 409 in the first CH3 domain with tryptophan and phenylalanine at position 405 in the second CH3 domain with lysine;

substitution of serine at position 364 in the first CH3 domain with lysine and leucine at position 368 in the second CH3 domain with aspartic acid, and substitution of lysine at position 409 in the first CH3 domain with tryptophan and phenylalanine at position 405 in the second CH3 domain with arginine;

substitution of serine at position 364 in the first CH3 domain with lysine and lysine at position 370 in the second CH3 domain with serine, and substitution of lysine at position 409 in the first CH3 domain with tryptophan and phenylalanine at position 405 in the second CH3 domain with lysine; or

substitution of serine at position 364 in the first CH3 domain with lysine and lysine at position 370 in the second CH3 domain with serine, and substitution of lysine at position 409 in the first CH3 domain with tryptophan and phenylalanine at position 405 in the second CH3 domain with arginine.

Such a double mutation in the CH3 domain may result in a dimerization rate of 70% or higher, 75% or higher, 80% or higher, 85% or higher, 90% or higher, 92% or higher, 93% or higher, 94% or higher, 95% or higher, or 96% or higher.

Another aspect of the present invention provides an anti-influenza B antibody comprising a heavy chain variable region composed of the amino acid sequence of SEQ ID NO: 27 and a light chain variable region composed of the amino acid sequence of SEQ ID NO: 31, or an antigen-binding fragment thereof. Another aspect of the present invention provides an anti-influenza A antibody comprising a heavy chain variable region composed of the amino acid sequence of SEQ ID NO: 29 and a light chain variable region composed of the amino acid sequence of SEQ ID NO: 31, or an antigen-binding fragment thereof.

Another aspect of the present invention provides an anti-influenza A/anti-influenza B bispecific antibody comprising an anti-influenza B antibody comprising a heavy chain variable region composed of the amino acid sequence of SEQ ID NO: 27 and a light chain variable region composed of the amino acid sequence of SEQ ID NO: 31, and an anti-influenza A antibody comprising a heavy chain variable region composed of the amino acid sequence of SEQ ID NO: 29 and a light chain variable region composed of the amino acid sequence of SEQ ID NO: 31, or an antigen-binding fragment thereof. The anti-influenza A/anti-influenza B bispecific antibody may comprise (1) the modified CH3 domain (as mentioned above, a mutation pair of CH3-CH3 introduced); (2) the mutant CH1 domain and the mutant CL domain (as mentioned above, a mutation pair of CH1-CL introduced); or (3) both the modified CH3 domain, and the mutant CH1 domain and the mutant CL domain.

The bispecific protein or bispecific antibody of the present invention is a bispecific matter constructed according to the Correlated and Harmonious Interfacial Mutation between Protein Subunits (hereinafter referred to as “Chimps”).

As used herein, the term “antibody” refers to a class of structurally related glycoproteins consisting of two pairs of polypeptide chains, one pair of light (L) low molecular weight chains and one pair of heavy (H) chains, all four inter-connected by disulfide bonds. The structure of antibodies has been well characterized (see, for instance, [Fundamental Immunology Ch. 7 (Paul, W., 2^{nd} ed. Raven Press, N. Y. (1989)]). In brief, each heavy chain typically is comprised of a heavy chain variable region (abbreviated herein as VH) and a heavy chain constant region. The heavy chain constant region typically is comprised of three domains, CH1, CH2, and CH3. The heavy chains are inter-connected via disulfide bonds in the so-called “hinge region”. Each light chain typically is comprised of a light chain variable region (abbreviated herein as VL) and a light chain constant region. The light chain constant region typically is comprised of one domain, CL. Typically, the numbering of amino acid residues in the constant region is performed according to the EU-index as described in the document [Kabat et al., Sequences of Proteins of Immunological Interest, 5th Ed. Public Health Service, National Institutes of Health, Bethesda, Md. (1991)]. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four 1-Rs, arranged from amino-terminus to carboxy-terminus in the following order: 1-R1, CDR1, FR2, CDR2, FR3, CDR3, FR4 (see also document [Chothia and Lesk J. Mol. Biol. 196, 901 917 (1987)]).

As used herein, the term “Fab-arm” refers to one heavy chain-light chain pair.

The term “Fc region”, as used herein, refers to an antibody region comprising a CH2 domain and a CH3 domain and may further comprise the hinge region, optionally.

As used herein, the term “bispecific antibody” refers to an antibody having specificities for at least two different epitope, typically non-overlapping epitopes.

The term “full-length antibody”, as used herein, refers to an antibody which contains all heavy and light chain constant and variable domains corresponding to those that are normally found in an antibody of that isotype. In an embodiment, a full-length antibody comprises two full-length heavy chains and two full-length light chains. As used herein, “isotype” refers to the antibody class (for instance IgG1, IgG2, IgG3, IgG4, IgD, IgA, IgE, or IgM) that is encoded by heavy chain constant region genes.

The term “antigen-binding fragment” is intended to mean a portion of an antibody containing variable domains of the antibody and may be selected from among Fab, F(ab′)2, scFv, (scFv)2, scFv-Fc, (scFv-Fc), etc., but is not limited thereto.

The term “epitope” means a protein determinant capable of specifically binding to an antibody. Epitopes usually consist of surface groupings of molecules such as amino acids or sugar side chains and usually have specific three dimensional structural characteristics as well as specific charge characteristics.

The term “bispecific antibody” is generic to the antibody that recognizes and/or binds to two different antigens or two different (non-overlapping) epitopes on one antigen. In an embodiment, the bispecific antibody may comprise one antigen-binding site directed against a tumor cell antigen and another antigen-binding site directed against a cytotoxic trigger molecule. By way of example, the bispecific antibody may be selected from the group consisting of an anti-FcγRI/anti-CD15 antibody, an anti-p185HER2/anti-FcγRBI (CD16) antibody, an anti-CD3/anti-malignant B-cell (1D10) antibody, an anti-CD 3/anti-p185HER2 antibody, an anti-CD3/anti-p97 antibody, an anti-CD3/anti-renal cell carcinoma antibody, an anti-CD3/anti-OVCAR-3 antibody, an anti-CD3/anti-L-D1 (anti-colon cancer) antibody, an anti-CD3/anti-melanocyte stimulating hormone analog antibody, an anti-EGF receptor/anti-CD3 antibody, an anti-CD3/anti-CAMA1 antibody, an anti-CD3/anti-CD19 antibody, an anti-CD3/MoV18 antibody, an anti-neural cell adhesion molecule (NCAM)/anti-CD3 antibody, an anti-folate binding protein (FBP)/anti-CD3 antibody, an anti-pan carcinoma associated antigen (AMOC-31)/anti-CD3 antibody, but are not limited thereto. In another embodiment, bispecific antibodies with one antigen-binding site which binds specifically to a tumor antigen and another antigen-binding site which binds to a toxin may comprise, but is not limited to, an anti-saporin/anti-Id-1 antibody, an anti-CD22/anti-saporin antibody, an anti-CD7/anti-saporin antibody, an anti-CD38/anti-saporin antibody, an anti-CEA/anti-ricin A chain antibody, an anti-interferon-α (IFN-α)/anti-hybridoma idiotype antibody, and an anti-CEA/anti-vinca alkaloid antibody. In another embodiment, the bispecific antibody may be selected from among antibodies for converting enzyme activated prodrugs such as anti-CD30/anti-alkaline phosphatase (which catalyzes conversion of mitomycin phosphate prodrug to mitomycin alcohol), but is not limited thereto. In another embodiment, the bispecific antibody may be selected from among those that can be used as fibrinolytic agents such as anti-fibrin/anti-tissue plasminogen activator (tPA), anti-fibrin/anti-urokinase-type plasminogen activator (uPA), etc., but is not limited thereto. In another embodiment, the bispecific antibody may be selected from among those for targeting immune complexes to cell surface receptors such as anti-low density lipoprotein (LDL)/anti-Fc receptor (e.g. FcγRI, FcγRII or FcγRBIE), but is not limited thereto. In another embodiment, the bispecific antibody may be selected from those for use in therapy of infectious diseases (e.g., viral infection diseases) such as anti-influenza A/anti-influenza B, anti-CD3/anti-herpes simplex virus (HSV), anti-T-cell receptor:CD3 complex/anti-influenza, anti-FcγR/anti-HIV, etc., but is not limited thereto. In another embodiment, the bispecific antibody may be selected from those for tumor detection in vitro or in vivo such as anti-CEA/anti-EOTUBE, anti-CEA/anti-DPTA, anti-p185 HER2/anti-hapten, etc., but is not limited thereto. In another embodiment, the bispecific antibody may be selected from those for use as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase, etc. In another embodiment, examples of the bispecific antibody comprise, but are not limited to, an antibody inclusive of a first antigen-binding site directed against CD30 and an antigen-binding site directed against erbB2; an antibody inclusive of a first antigen-binding site directed against CD30 and a second antigen-binding site directed against Pseudomonas exotoxin (PE); an antibody inclusive of a first antigen-binding site directed against CD30 and a second antigen-binding site directed against streptavidin.

According to another embodiment, the targeting domain of the bispecific protein may comprise at least one target specific binding polypeptide selected from the group consisting of various membrane proteins, for example, various receptors (e.g., receptor tyrosine kinase (RTKs), etc.), ectodomains (extracellular domains) of the receptors, and various ligands (e.g., various growth factors, cytokines, etc.). Examples of the receptor comprise, but are not limited to, tumor necrosis factor receptor (TNFR) (e.g., TNFR1, TNFR2, etc.), epidermal growth factor receptor (EGFR) (e.g., Her1 (epidermal growth factor receptor 1), Her2 (human epidermal growth factor receptor 2), Her3 (human epidermal growth factor receptor 3), etc.), angiopoietin receptor (e.g., Tie1, Tie2, etc.), transforming growth factor receptor (e.g, TGFbR1, TGFbR2, TGFbR3, TGFaR1, etc.), bone morphogenetic protein receptor (e.g, BMPR1b), interleukin receptor (e.g., interleukin 12 receptor subunit beta 1 (IL-12R-b1)), IL-4Ra, IL-12A, IL-4, IL-1R1L, IL-17RA, IL-17A, IL-12R-b2, IL-13Ra1, IL-12B, IL-13, IL-1RAP, IL-17RC, IL-17F, etc.), integrin (e.g., integrin alpha 4 (ITGA4), integrin subunit alpha 2b (ITGA2B), ITGB1, ITGB3, etc.), interferon receptor (e.g., interferon-alpha/beta receptor 1 (IFNAR1), IFNAR2, IFNGR, etc.), Fas (tumor necrosis factor receptor superfamily member 6; TN1RSF6), VEGF receptor (e.g., Flt1 (fins related tyrosine kinase 1), etc.), hepatocyte growth factor receptor (e.g., Met, etc.), and Interferon gamma receptor (IFNGR). The ligand may be at least one selected from the group consisting of tumor necrosis factor (TNF), epidermal growth factor (EGF), vascular endothelial cell growth factor (VEGF-A, VEGF-B, VEGF-C, VEGF-D, etc.), angiopoietin (e.g., Ang1, Ang2, etc.), transforming growth factor (TGF), hepatocyte growth factor (HGF), bone morphogenetic protein (e.g., BMP2, BMP7, etc.), interleukin, and interferon, but is not limited thereto.

The term “host cell”, as used herein, is intended to refer to a cell into which an expression vector has been introduced, e.g. an expression vector encoding an antibody of the invention. Recombinant host cells comprise, for example, transfectomas, such as CHO cells, HEK293 cells, NS/0 cells, and lymphocytic cells.

CL domains, CH1 domains, and Fc regions (e.g., CH3 domains) in the antibodies of the present invention may be obtained from any antibody such as IgG1, IgG2, IgG3, or IgG4 subtype, IgA, IgE, IgD, or IgM. The antibodies may be derived from mammals comprising primates such as humans, monkeys, etc. and rodents such as mice, rats, etc. Because antibodies derived from mammals exhibit high sequence homology and structural homology among species, an explanation given of the CL domain, CH1 domain, and CH3 domain in the description is generally applicable to antibodies derived from mammals. In one embodiment, the CL domain, CH1 domain, and CH3 domain may be derived from IgG (e.g., IgG1), but is not limited thereto. As mentioned above, the Fc region in the antibodies described in the present invention comprise two different heavy chains (e.g., different in the sequence of variable domain) Of the two different heavy chains, at least one undergoes an amino acid mutation to increase a possibility of stably forming a heterodimer between the two different heavy chains, but decrease a possibility of stably forming a homodimer between two identical heavy chains.

Bispecific proteins, bispecific antibodies, and antigen-binding fragments thereof provided in the description can be constructed using any means and, for example, by a chemical synthesis or recombinant method. The proteins, the antibodies, and the fragments may be non-naturally occurring.

Mutation sites necessary for dimerization of Fc in the present invention are depicted in FIG. 2. Targets amount to 39 sites for electrostatic interaction-introduced mutation and 14 sites for swapping mutation, and 40 sites for size mutation. In FIG. 2, amino acid residues are expressed as capital letters according to a typical method. The numbering of amino acid residues in the constant region is performed according to the EU-index as described in the document [Kabat et al., Sequences of Proteins of Immunological Interest, 5th Ed. Public Health Service, National Institutes of Health, Bethesda, Md. (1991)].

Another aspect of the present invention provides a pharmaceutical composition comprising the aforementioned bispecific protein or bispecific antibody and optionally a pharmaceutically acceptable carrier. Another aspect provides a use of the aforementioned bispecific protein or bispecific antibody in preparing a pharmaceutical composition. Another aspect provides a method for preparing a pharmaceutical composition comprising the aforementioned bispecific protein or bispecific antibody.

An antibody and a composition comprising the same (e.g., pharmaceutical composition) can be applied to diagnosis and treatment, and as such, can be contained in a therapeutic or diagnostic kit.

As used herein, “pharmaceutically acceptable carrier” comprises any and all physiologically compatible solvents, dispersion media, coatings, antibacterial and antifungal agents, isotonicity agents, absorption delaying agents, typical vehicles used for preparation of other drugs, excipients, and additives. Preferably, the carrier may be suitable for intravenous, intramuscular, subcutaneous, parenteral, intraspinal, or epidermal administration (e.g., by injection or infusion).

A composition of the present invention can be administered by a variety of methods known in the art. As will be appreciated by a person skilled in the art, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents comprise saline and aqueous buffer solutions. Pharmaceutical carriers comprise sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally”, as used herein, means modes of administration other than enteral and topical administration, usually by injection, and comprises intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to comprise isotonic agents, such as sugars, sodium chloride, and the like into the compositions. In addition, prolonged absorption of the injectable pharmaceutical form may be brought about by the inclusion of agents which delay absorption such as aluminum monostearate and gelatin. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors comprising the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The administration subject may be selected from among mammals comprising primates such as humans, monkeys, etc., rodents such as mice, rats, etc., and the like, isolates therefrom comprising cells, tissues, and body fluid (e.g., blood, etc.), and cultured products thereof.

### Advantageous Effects

The present invention provide a highly pure heterodimeric protein as a Chimps protein (e.g., antibody), which is significantly free of contaminants such as homodimers or monomers, and a construction technique therefor. Another advantage of the present invention is to increase the purity of the bispecific antibody and to introduce a minimal number of mutations to a natural antibody, thereby causing no significant structural changes of natural antibodies and reducing the risk of inducing the antibody to undergo functional loss or abnormality and/or to elicit immune rejection.

### DESCRIPTION OF DRAWINGS

FIG. 1 is a schematic diagram showing only one perfect heterodimeric bispecific antibody (dotted line circle) among a variety of antibody forms possible for construction of bispecific antibodies (10 in total). A and B represent respective heavy chains different from each other and a and b represent respective light chain different from each other.

FIG. 2 shows residue positions of antibodies used in electrostatic interaction, swapping, and size methods for inducing heterodimerization in Fc constant regions according to one embodiment, and residue positions related thereto.

FIG. 3 is an SDS-PAGE profile showing heterodimerization results from electrostatic interaction-induced mutations listed in Table 4.

FIG. 4 is an SDS-PAGE profile showing heterodimerization results from swapping-associated mutations listed in Table 5.

FIG. 5 is an SDS-PAGE profile showing heterodimerization results from size-associated mutations listed in Table 6.

FIG. 6a shows comparison of 12 mutations that allow outstanding heterodimerization among the single mutations lusted in Table 7.

FIG. 6b shows heterodimerization results after S364K of two key mutations was substituted with other amino acids according to one embodiment.

FIG. 6c shows heterodimerization results after F405K of two key mutations was substituted with other amino acids according to one embodiment.

FIG. 6d shows heterodimerization results after additional mutations were introduced to the three selected key-lock mutation pairs S364K-L368D, S364K-K370S, and F405K-K409F according to one embodiment, wherein x-axis accounts for molecular weights (kD).

FIG. 7 shows heterodimerization efficiency of the single mutations S364K-L368D, S364K-K370S, and F405K-K409F in comparison with conventional techniques (KiH, CPC, and AzS controls). When amino acid residues corresponding to each key are changed to other different amino acids, K is best for S364 and K and R (arginine) show almost the same effect for F405. Positions at which single mutations are made on A and B chains are indicated and heterodimerization quantity is numerically expressed.

FIG. 8 shows heterodimization efficiencies of a total of four double mutation pairs in comparison with conventional techniques (KiH, CPC, AzS controls), wherein the four double mutations are obtained by selecting two double mutation pairs from combinations of the three mutation pairs S364K-L368D, S364K-K370S, and F405K-K409F and mutating F405 into two types K and R for each pair.

FIGS. 9a to 9c are SDS-PAGE profiles after L368, K370, and K409 corresponding to lock mutations in double mutation pairs are substituted with other amino acids as indicated in Table 6 in order to identify better effects when amino acid residues corresponding to lock mutations are changed to other mutations.

FIG. 10 shows positions of electrostatic interaction-associated mutations, size-associated mutations, and swap-associated mutations in Fab of antibodies.

FIG. 11 is a schematic diagram of a competitive pairing (CPP) assay procedure.

FIG. 12 shows pairing modes established through the process of FIG. 11 between heavy and light chains and visualized on SDS-PAGE after 4D9 and 2B9 having conventional mutation pairs Cl, DuetMab, and V23 between heavy and light chains were cloned and co-expressed.

FIG. 13 is an SDS-PAGE profile showing pairing modes when an electrostatic interaction-associated mutation between heavy and light chains is established by substituting K(lysine) for target amino acids on A chain (2B9 heavy chain) and D (aspartic acid) for target amino acids on B chain (4D9 heavy chain).

FIG. 14 shows pairing modes of the mutation at position 30 resulting from substituting heavy chain L145 and light chain V133 with K and D, respectively, which exhibit relatively high pairing accuracy among the list of mutation pairs as the 4D9 and 2B9 antibodies are compared on SDS-PAGE.

FIGS. 15 and 16 show pairing modes after mutation at position 29 S131D and/or S131K is introduced to the light chain of the antibody in which heavy chain L145 is substituted with E or D and light chain V133 is substituted with R, as analyzed by SDS-PAGE.

FIG. 17 shows pairing modes of mutation pairs at position 48 (heavy chain S183 and light chain V133 were substituted with K(R) and D(E), respectively), as analyzed by SDS-PAGE.

FIG. 18 shows pairing modes analyzed by SDS-PAGE after the mutation pair c29c30c48FΦ29f30f48 shown in Table 19 and variations thereof are introduced.

FIG. 19 shows pairing modes analyzed by SDS-PAGE after the mutation pairs of Table 20 are introduced.

FIG. 20 shows pairing modes analyzed by SDS-PAGE after the mutation pairs of Table 21 are introduced.

FIG. 21 shows paring ratios of chains after combinations of the mutation pairs at position 34 and 51 selected from among the heavy and light chain mutation pairs are introduced according to one embodiment.

FIG. 22 shows paring ratios of chains after c34Φf51 mutation pair selected from among the heavy and light chain mutation pairs is introduced according to one embodiment.

FIG. 23 shows paring ratios of chains after c40Φf44 mutation pair selected from among the heavy and light chain mutation pairs is introduced according to one embodiment.

FIG. 24 is a schematic diagram of a bispecific antibody in which the heavy and light chains are mutated according to one embodiment.

FIG. 25 is a graph showing thermal stability of A chain and B chain for the heavy chain and a chain and b chain for the light chain in antibodies constructed according to one embodiment.

FIG. 26 is a cleavage map of pcDNA3.

FIG. 27 is a graph showing hydrophobic interaction chromatography (HIC) results of the bispecific antibodies Trabev and Adabev constructed according to one embodiment, each having c′29c′30c48Φf′29f′30f′48 mutation pair and AWBB mutation pair introduced thereto.

FIG. 28 is a graph showing size exclusion chromatography (SEC) analysis results of the bispecific antibody Trabev, constructed according to one embodiment, having c′29c′30c48Φf′29f′30f′48 mutation pair and AWBB mutation pair introduced thereto.

FIG. 29 is a graph showing size exclusion chromatography (SEC) analysis results of the bispecific antibodies Trabev and Adabev constructed according to one embodiment, each having c′29c′30c48Φf′29f′30f′48 mutation pair and AWBB mutation pair introduced thereto.

FIG. 30 shows dimerization modes of the bispecific antibodies Trabev and Adabev constructed according to one embodiment, each having c′29c′30c48Φf′29f′30f′48 mutation pair and AWBB mutation pair introduced thereto.

FIG. 31 is a graph showing affinity for antigens (Her2 and VEGF) of the bispecific antibody Trabev, constructed according to one embodiment, having c′29c′30c48Φf′29f′30f′48 mutation pair and AWBB mutation pair introduced thereto.

FIG. 32 is a graph showing of for antigens (TNF-alpha and VEGF) of the bispecific antibody Adabev, constructed according to one embodiment, having c′29c′30c48Φf′29f′30f′48 mutation pair and AWBB mutation pair introduced thereto.

FIG. 33a shows sequence alignment between the human IgG1 heavy chain constant region and the human IgA1 heavy chain constant region (human IgG1 heavy chain constant region: SEQ ID NO: 33; human IgA1 heavy chain constant region: SEQ ID NO: 34).

FIG. 33b shows sequence alignment between the kappa constant region and lambda constant region of human immunoglobulin light chain (kappa constant region of human immunoglobulin light chain: SEQ ID NO: 35; and lambda constant region of human immunoglobulin light chain: SEQ ID NO: 36).

FIGS. 33c and 33d show sequence alignment of heavy chain constant regions among human, mouse, and rat IgG subtypes (CH1 domain sequences in FIG. 33c and CH3 domain sequences in FIG. 33d) (Human IgG1: SEQ ID NO: 1, Human IgG2: SEQ ID NO: 2, Human IgG3: SEQ ID NO: 3, Human IgG4: SEQ ID NO: 4, Mouse IgG1: SEQ ID NO: 37, Mouse IgG2a^{b}: SEQ ID NO: 38, Mouse IgG2a^{a}: SEQ ID NO: 39, Mouse IgG2b: SEQ ID NO: 40, Mouse IgG3: SEQ ID NO: 41, Rat IgG1: SEQ ID NO: 42, Rat IgG2a: SEQ ID NO: 43, Rat IgG2b: SEQ ID NO: 44, Rat IgG2c: SEQ ID NO: 45 in FIG. 33c (CH1 domain); and Human IgG1: SEQ ID NO: 15, Human IgG2: SEQ ID NO: 16, Human IgG3: SEQ ID NO: 17, Human IgG4: SEQ ID NO: 18, Mouse IgG1: SEQ ID NO: 46, Mouse IgG2a^{b}: SEQ ID NO: 47, Mouse IgG2a^{a}: SEQ ID NO: 48, Mouse IgG2b: SEQ ID NO: 49, Mouse IgG3: SEQ ID NO: 50, Rat IgG1: SEQ ID NO: 51, Rat IgG2a: SEQ ID NO: 52, Rat IgG2b: SEQ ID NO: 53, and Rat IgG2c: SEQ ID NO: 54 in FIG. 33d (CH3 domain)).

FIG. 34 shows paring levels between light and heavy chains in antibodies, constructed according to one embodiment, having c34Φf51 mutation pair introduced thereto.

FIG. 35 is a graph showing a HIC result of the bispecific antibody Trabev, constructed according to one embodiment, having c34Φf51 mutation pair and AWBB mutation pair.

FIG. 36 shows graphs of HIC results of the bispecific antibody Adabev, constructed according to one embodiment, having c34Φf51 mutation pair and AWBB mutation pair.

FIG. 37 shows dimerization modes of the bispecific antibodies Trabev and Adabev, constructed according to one embodiment, each having c34Φf51 mutation pair and AWBB mutation pair introduced thereto.

FIG. 38 shows pairing levels between light and heavy chains in an antibody, constructed according to one embodiment, having c40Φf44 mutation pair introduced thereto.

FIG. 39 shows graphs of HIC results of the bispecific antibody Trabev, constructed according to one embodiment, having c40Φf44 mutation pair and AWBB mutation pair.

FIG. 40 shows graphs of HIC results of the bispecific antibody Adabev, constructed according to one embodiment, having c40Φf44 mutation pair and AWBB mutation pair.

FIG. 41 shows dimerization modes of the bispecific antibodies Trabev and Adabev, constructed according to one embodiment, each having c40Φf44 mutation pair and AWBB mutation pair introduced thereto.

FIG. 42 is an SDS-PAGE profile showing homodimerization levels upon single transfection of Enb-Fc and Fas-Fc, separately, according to a Comparative Example.

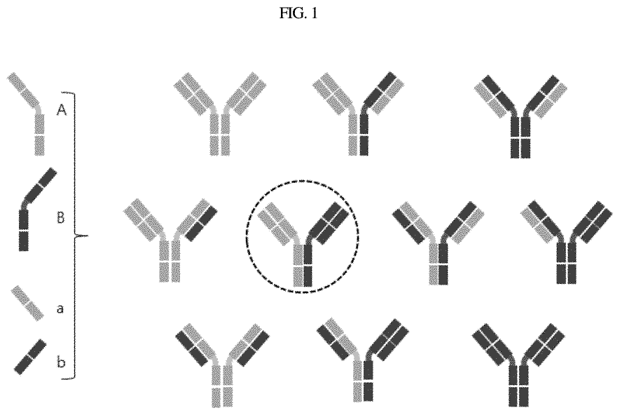

### MODE FOR INVENTION

Hereinafter, the present invention will be described in further detail with reference to examples. It is to be understood, however, that these examples are for illustrative purposes only and are not to be construed to limit the scope of the present invention.

For the practice of the present invention, all samples were prepared after the following procedure.

Protein Expression

1. A target gene was cloned to an expression vector (pcDNA3 (Invitrogen).

2. HEK293E cells (ATCC) were cultured in high-glucose DMEM (Dulbecco's modified Eagle's medium) supplemented with 5% PBS (fetal bovine serum) in a humidified, CO₂ incubator.

3. A prepared plasmid DNA was introduced by transient transfection into HEK393E cells grown to full confluency. Before transfection, the cells were washed with PBS (phosphate-buffered saline) and, followed by exchanging the culture medium with serum-free high-glucose DMEM.

4. After incubation for one week, a conditioned medium was used to harvest the proteins which were then filtered. Fc-fusion proteins and antibodies were isolated by protein A chromatography.

5. Isolated proteins were quantitatively determined as analyzed at 280 nm.

### Example 1: Selection of Mutation for Heterodimerization of Two Fe Regions

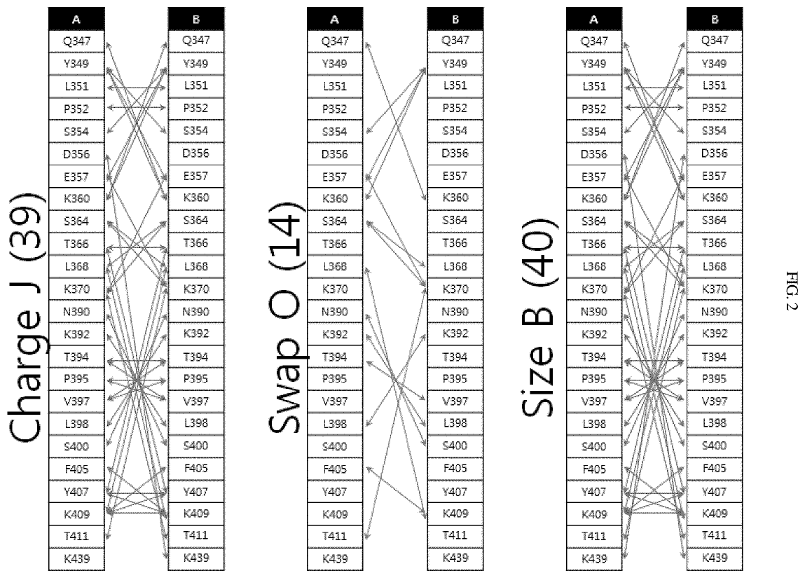

Mutation positions for heterodimerization of two Fc regions (CH3 domain; SEQ ID NO: 15) (on the basis of IgG1) are depicted in FIG. 2. Section was made of 39 positions for electrostatic interaction-associated mutation (electrostatic interaction-introduced mutation) (represented by “Charge J”), 14 positions for swapping-associated mutation (swapping mutation) (represented by “Swap 0”), and 40 positions for size-associated mutation (size mutation) (represented by “Size B”). Finally selected positions in CH3 domains and mutated amino acids thereat are summarized in Table 3. Each of the mutation pairs was applied two Fc regions (respectively represented by A chain and B chain) derived from different antibodies, followed by cloning for co-expression. For electrostatic interaction-associated mutation, mutations were carried out to substitute the corresponding amino acids on A chain with K (lysine) representative of amino acids having a positive charge and on B chain with D (aspartic acid) representative of amino acids having a negative charge. Swapping-associated mutation was carried out to exchange the corresponding amino acids on A chain and B chain with each other. For size-associated mutation, the corresponding amino acids were substituted with W (tryptophan) on A chain and with the small size amino acid A (alanine) on B chain

TABLE 3

| Amino acid | Charge (J) | | Swap (O) | | Size (B) | |
|---|---|---|---|---|---|---|
| pair No. | Chain A | Chain B | Chain A | Chain B | Chain A | Chain B |
| 1 | Q347K | K360D | Q347K | K360Q | Q347W | K360A |
| 2 | Y349K | S354D | | | Y349W | S354A |
| 3 | Y349K | E357 | | | Y349W | E357A |
| 4 | Y349K | K360D | | | Y349W | K360D |
| 5 | L351K | L351D | | | L351W | L351A |
| 6 | P352K | P352D | | | P352W | P352A |
| 7 | S354K | Y349D | S354Y | Y349S | S354W | Y349A |
| 8 | D356K | K439D | | | D356W | K439A |
| 9 | E357K | Y349D | E357Y | Y349E | E357W | Y349A |
| 10 | E357K | K370D | E357K | K370E | E357W | K370A |
| 11 | K360K | Q347D | | | K360W | Q347A |
| 12 | K360K | Y349D | K360Y | Y349K | K360W | Y349A |
| 13 | S364K | L368D | S364L | L368S | S364W | L368A |
| 14 | S364K | K370D | S364K | K370S | S364W | K370A |
| 15 | T366K | T366D | | | T366W | T366A |
| 16 | T366K | Y407D | | | T366W | Y407A |
| 17 | L368K | S364D | | | L368W | S364A |
| 18 | L368K | K409D | L368K | K409L | L368W | K409A |
| 19 | K370 | E357D | | | K370W | E357A |
| 20 | K370 | S364D | | | K370W | S364A |
| 21 | K370 | T411D | | | K370W | T411A |
| 22 | N390K | S400D | N390S | S400N | N390W | S400A |
| 23 | K392 | L398D | | | K392W | L398A |
| 24 | T394K | T394D | | | T394W | T394A |
| 25 | T394K | V397D | T394V | V397T | T394W | V397A |
| 26 | P395K | P395D | | | P395W | P395A |
| 27 | P395K | V397D | | | P395W | V397A |
| 28 | V397K | T394D | | | V397W | T394A |
| 29 | V397K | P395D | | | V397W | P395A |
| 30 | L398K | K392D | L398K | K392L | L398W | K392A |
| 31 | S400K | N390D | | | S400W | N390A |
| 32 | F405K | K409D | F405K | K409F | F405W | K409A |
| 33 | Y407K | T366D | Y407T | T366Y | Y407W | T366A |
| 34 | Y407K | Y407D | | | Y407W | Y407A |
| 35 | Y407K | K409D | | | Y407W | K409A |
| 36 | K409 | L368D | | | K409W | L368A |
| 37 | K409 | F405D | | | K409W | F405A |
| 38 | K409 | Y407D | | | K409W | Y407A |
| 39 | T411K | K370D | T411K | K370T | T411W | K370A |
| 40 | | | | | K439W | D356A |

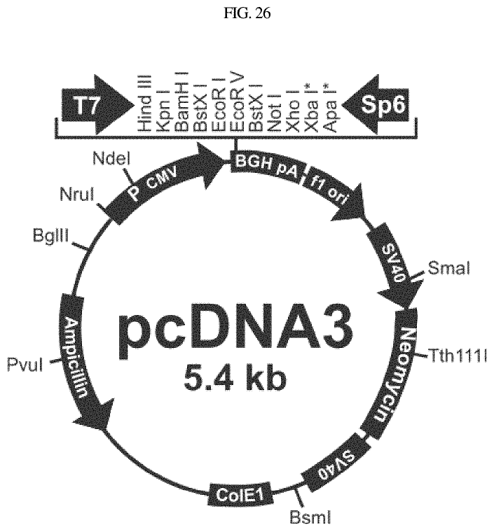

In order to easily identify homodimers or heterodimers of Fc-fusion proteins through SDS-PAGE, fusion was made of the ectodomain (coding sequence of region 1-771 of SEQ ID NO: 24) of TNF-alpha receptor (TNFRSF1B: NP_001057.1 (coding gene: CDS of NM_001066.2; SEQ ID NO: 24)) to Fc (coding gene: SEQ ID NO: 26) on one chain (chain A: Enbrel) and the ectodomain (coding sequence of region 1-519 of SEQ ID NO: 25) of Fas receptor (NP_000034.1 (coding gene: CDS of NM_000043.5; SEQ ID NO: 25)) to Fc (coding gene: SEQ ID NO: 26) on the other chain (chain B: Fas), using pcDNA3 vector (see FIG. 26; Invitrogen) as a backbone. The monomer of chain A has a size of 53 kD while the monomer of chain B has a size of about 44 kD. Because chains A and B, each having an Fc-ectodomain fusion, were different in size from each other, two homodimers (AA and BB) and one heterodimer (AB) could be easily discriminated on SDS-PAGE.

Percentages (%) of dimerization between chains A and between chains B (homodimerization) are expressed as S_{AA} and S_{BB}, respectively whereas S accounts for percentages (%) of dimerization between chains A and B (heterodimerization).

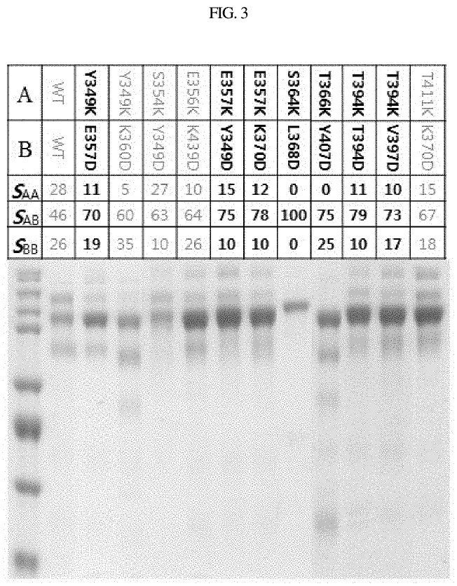

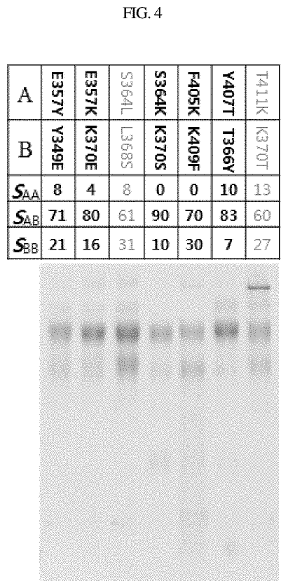

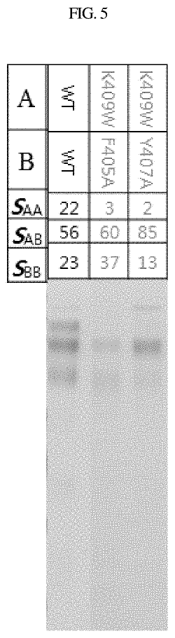

When modes of dimerization were observed by SDS-PAGE, ratios (%) of homodimerization (AA and BB) and heterodimerization (AB) between chains A and B having the mutations of Table 3 introduced thereinto were compared with those between chains A and B that had not been mutated (represented by WT). Results are given Table 4 and FIG. 3 for the electrostatic interaction-induced mutation, Table 5 and FIG. 4 for the size mutation, and Table 6 and FIG. 5 for the swapping-associated mutation.

TABLE 4

| A | WT | Y349K | Y349K | S354K | E356K | E357K | E357K | S364K | T366K | T394K | T394K | T411K |
|---|---|---|---|---|---|---|---|---|---|---|---|---|
| B | WT | E357D | K360D | Y349D | K439D | Y349D | K370D | L368D | Y407D | T394D | V397D | K370D |
| S_{AA} | 28 | 11 | 5 | 27 | 10 | 15 | 12 | 0 | 0 | 11 | 10 | 15 |
| S_{AB} | 46 | 70 | 60 | 63 | 64 | 75 | 78 | 100 | 75 | 79 | 73 | 67 |
| S_{BB} | 26 | 19 | 35 | 10 | 26 | 10 | 10 | 0 | 25 | 10 | 17 | 18 |

TABLE 5

| A | E357Y | E357K | S364L | S364K | F405K | Y407T | T411K |
|---|---|---|---|---|---|---|---|
| B | Y349E | K370E | L368S | K370S | K409F | T366Y | K370T |
| S_{AA} | 8 | 4 | 8 | 0 | 0 | 10 | 13 |
| S_{AB} | 71 | 80 | 61 | 90 | 70 | 83 | 60 |
| S_{BB} | 21 | 16 | 31 | 10 | 30 | 7 | 27 |

TABLE 6

| A | WT | K409W | K409W |
|---|---|---|---|
| B | WT | F405A | Y407A |
| S_{AA} | 22 | 3 | 2 |
| S_{AB} | 56 | 60 | 85 |
| S_{BB} | 23 | 37 | 13 |

In Tables 4 to 6, mutations accounting for a heterodimerization rate (%) of 70% or higher are expressed in bold. As is understood from data of Tables 4 to 6, the tested mutation pairs exhibited a heterodimerization rate of 60% or higher.

Through the results, selection was made of 12 mutation pairs that gave higher rates to heterodimerization than homodimerization, with the heterodimerization rate being 70% or higher (electrostatic interaction-introduced mutation: Y349K-E357D, E357K-Y349D, E357K-K370D, S364K-L368D, T366K-Y407D, T394K-T394D, T394K-V397D; and swapping mutation: E357Y-Y349E, E357K-K370E, S364K-K370S, F405K-K409F, Y407T-T366Y) (expressed in bold in Tables 4 and 5).

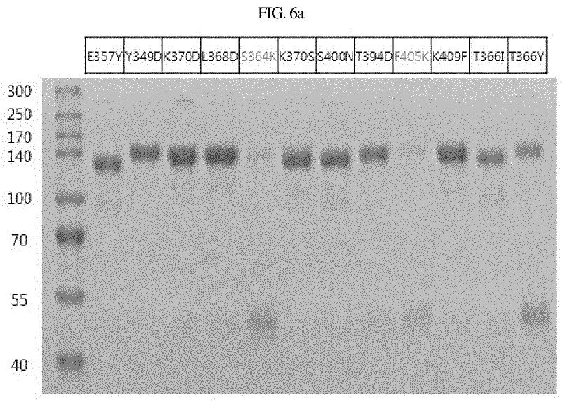

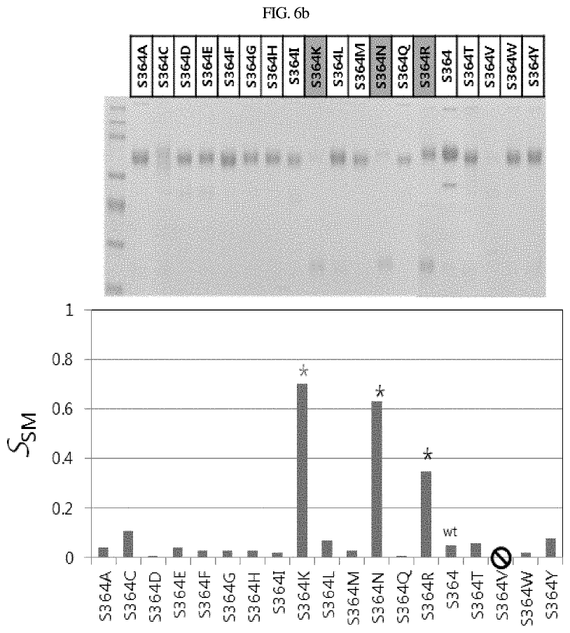

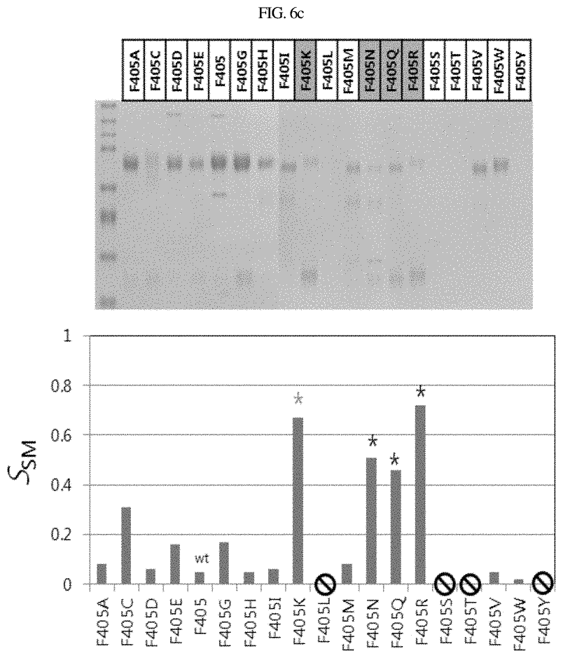

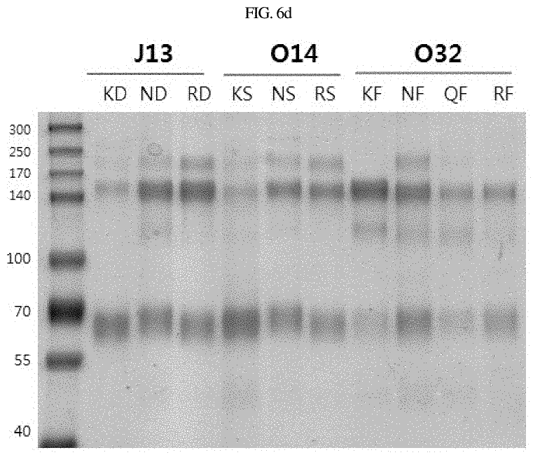

Each of the 12 amino acid residue mutations contained in the selected 12 amino acid pairs was introduced into the Fas-Fc fusion protein to express mutant Fas-Fc fusion proteins that contained single mutations at the amino acid positions, respectively. In the same condition, homodimer and monomer survival rates (Sₛₘ; 1=100%) were compared, and the results are depicted in Table 7 and FIG. 6a.

TABLE 7

| SM | S_{SM} |
|---|---|
| E357Y | 0.03 |
| Y349D | 0.03 |
| K370D | 0.06 |
| L368D | 0.06 |
| S364K | 0.77 |
| K370S | 0.05 |
| S400N | 0.05 |
| T394D | 0.11 |
| F405K | 0.67 |
| K409F | 0.05 |
| T366A | 0.09 |
| T366Y | 0.46 |

In Table 7 and FIG. 6a, two mutations S364K and F405K, which were low in homodimerization rate but high in monomer survival rate (50% or higher), were selected.

It was postulated that the two selected mutations acted as “key” mutations while the CH3 domain sites interacting therewith on the other chain were “lock” mutations. As combinations therebetween, three key-lock pairs S364K-L368D, S364K-K370S, and F405K-K409F were selected. These mutation pairs were introduced into chain A (key mutation) and chain B (lock mutation) to make three different single mutation pairs (see TABLE 8).

TABLE 8

| | | Key Single | Lock Single |
|---|---|---|---|
| Mutation type | Sample | Mutation | Mutation |
| Introduction of | J13 | S364K | L368D |
| electrostatic interaction | | | |
| Swapping | O14 | S364K | K370S |
| Swapping | O32 | F405K | K409F |

The amino acids at respective key mutation positions were substituted with different amino acid residues and tested for the single mutation-induced heterodimerization effect, as described above, so as to identify mutation types effective for heterodimerization at the positions. For S364, substitution with K (lysine) was observed to bring about the highest heterodimerization effect. An outstanding effect was detected upon substitution with N (asparagine) and R (arginine) (see FIG. 6b). F405 exhibited excellent similar heterodimerization effects upon substitution with K and R and outstanding heterodimerization effects upon substitution with N and Q (glutamine) (see FIG. 6c).

The additional mutations S364N, S364R, F405R, F405N, and F405Q, which were identified in FIGS. 6b and 6c, were applied to the three selected key-lock mutation pairs S364K-L368D, S364K-K370S, and F405K-K409F, to introduce the lock mutations to chain A (TNFR2-Fc) and the key mutations to chain B (Fas-Fc), followed by analyzing heterodimerization effects on SDS-PAGE.

The results thus obtained are depicted in Table 9 and FIG. 6d:

TABLE 9

| | A(TNFR2) | B(Fas) | | | |
|---|---|---|---|---|---|
| | Key Single | Lock Single | | | |
| Sample | Mutation | Mutation | S_{AA} | S_{AB} | S_{BB} |
| J13(K:D) | S364K | L368D | 8 | 92 | 0 |
| J13(N:D) | S364N | L368D | 8 | 82 | 9 |
| J13(R:D) | S364R | L368D | 20 | 79 | 1 |
| O14(K:S) | S364K | K370S | 25 | 65 | 9 |
| O14(N:S) | S364N | K370S | 13 | 84 | 3 |
| O14(R:S) | S364R | K370S | 23 | 76 | 1 |
| O32(K:F) | F405K | K409F | 0 | 75 | 25 |
| O32(N:F) | F405N | K409F | 21 | 62 | 17 |
| O32(Q:F) | F405Q | K409F | 5 | 68 | 27 |
| O32(R:F) | F405R | K409F | 5 | 89 | 6 |

(S_{AA}: AA homodimerization rate (%);
S_{AB}: AB heterodimerization rate (%);
S_{BB}: BB homodimerization rate (%))

### Example 2: Test for Heterodimerization of Fe Region by Single Mutation

The three key-lock mutation pairs S364K-L368D, S364K-K370S, and F405K-K409F, which were selected in Example 1, and the mutation pair F405R-K409F, which resulted from substituting F405 with R instead of K, were tested for heterodimerization on SDS-PAGE. The heterodimerization effects were compared with those obtained with the conventional heterodimer Fc mutation pairs KiH, CPC, and AzS used as controls.

SDS-PAGE data in this Example and all the following Examples were obtained by quantitating the band intensities with the aid of GelQuant.NET Software.

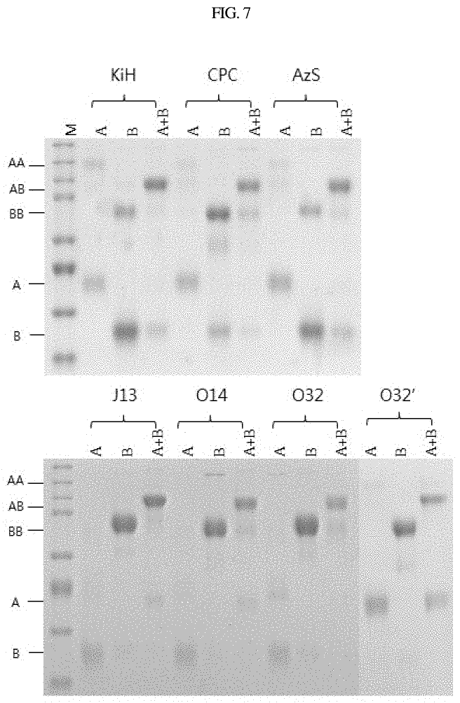

The results are depicted in Table 10 and FIG. 7:

TABLE 10

| | Chain A | | | | | | |
|---|---|---|---|---|---|---|---|
| Sample | (eTNFR2) | Chain B (eFas) | S_{AB} | S_{A} | S_{B} | S_{M} | Tₘ |
| J13 | S364K | L368D | 0.93 | 0.86 | 0.10 | 0.48 | 66.8 |
| O14 | S364K | K370S | 0.87 | 0.86 | 0.07 | 0.47 | 67.6 |
| O32 | F405K | K409F | 0.72 | 0.91 | 0.08 | 0.49 | 65.2 |
| O32′ | F405R | K409F | 0.72 | 0.91 | 0.08 | 0.49 | 65.2 |
| KiH | T366S/L368A/ | T366W | 0.90 | 0.68 | 0.64 | 0.66 | 67.4 |
| | Y407V | | | | | | |
| CPC | K392D/K409D | E356K/D399K | 0.74 | 0.85 | 0.30 | 0.58 | 66.4 |
| AzS | T350V/T366L/ | T350V/L351Y/ | 0.84 | 0.84 | 0.64 | 0.74 | 69.2 |
| | K392L/T394W | F405A/Y407V | | | | | |

(S_{AB}: AB heterodimerization rate (%);
S_{A}; AA homodimerization rate (%);
S_{B}: BB homodimerization rate (%);
S_{M}: monomer survival rate (%))

Tm was measured as follows:

Reagent: Invitrogen 4461146 “Protein Thermal Shift” Dye Kit

Instrument: Chromo4-PTC 200 (MJ Research)

Reaction mixture: 20 [symbol] in total

| | |
|---|---|
| Protein | 10 μl |
| DW | 3 μl |
| Protein Thermal Shift ™ Buffer | 5 μl |
| 1/100 diluted Protein Thermal Shift ™ Dye | 2 μl |

Protocol:

1. Incubate at 50.0□ for 30 sec;

2. Melting Curve from 50.0□ to 90.0□, read every 0.2□, hold for 2 sec;

3. Incubate at 90.0□ for 2 min

4. Incubate at 10.0□ forever

5. End

As shown in Table 10 and FIG. 7, when chain A and chain B were each separately expressed, chain A retaining the key did not form a homodimer, but is expressed as a monomer. A sample resulting from co-expression was observed to contain no homodimers, but mostly heterodimers (see FIG. 7).

### Example 3: Heterodimerization of Fe Region by Double Mutation

Less plausibility of homodimerization might result from existence of the key mutations on both of chains A and B than on either of the chains Two double mutation pairs were made from combinations of the three mutation pairs selected in Example 1. For each double mutation pair, F405 was mutated into two types K and R. Thus, a total of four double mutation pairs were obtained. These found double mutations pairs were analyzed for heterodimerization on SDS-PAGE and the dimerization effects were compared with those of the controls KiH, CPC, and AzS.

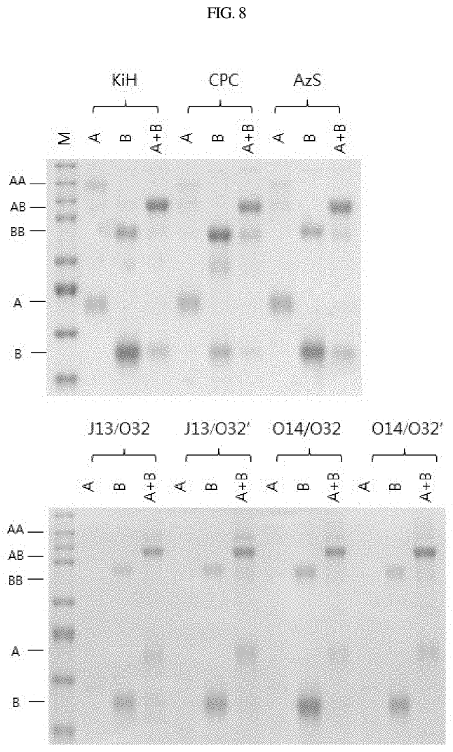

The results are given in Table 11 and FIG. 8:

TABLE 11

| | Chain A | | | | | | |
|---|---|---|---|---|---|---|---|
| DMP | (eTNFR2) | Chain B (eFas) | S_{AB} | S_{A} | S_{B} | S_{M} | Tₘ |
| J13/O32 | S364K/K409F | L368D/F405K | 0.96 | 1.00 | 0.74 | 0.87 | 58.2 |
| J13/O32′ | S364K/K409F | L368D/F405R | 0.95 | 1.00 | 0.73 | 0.87 | 61.5 |
| O14PO32 | S364K/K409F | K370S/F405K | 0.95 | 1.00 | 0.72 | 0.86 | 64.1 |
| O14/O32′ | S364K/K409F | K370S/F405R | 0.93 | 1.00 | 0.71 | 0.86 | 64.4 |
| KiH | T366S/L368A/Y407V | T366W | 0.91 | 0.64 | 0.61 | 0.63 | 67.4 |
| CPC | K392D/K409D | E356K/D399K | 0.73 | 0.81 | 0.30 | 0.58 | 66.4 |
| AzS | T350V/T366L/ | T350V/L351Y/ | 0.84 | 0.82 | 0.62 | 0.72 | 69.2 |
| | K392L/T394W | F405A/Y407V | | | | | |

As shown in Table 11 and FIG. 8, the key mutations present in both chains allowed the homodimerization of the chains for none of the four mutation pairs upon separate expression. All of the proteins in a sample resulting from co-expression were observed to be heterodimers (See FIG. 8). In addition, higher thermal stability was measured in a double-mutation pair having F405R introduced thereto than F405K introduced thereto (Table 11).

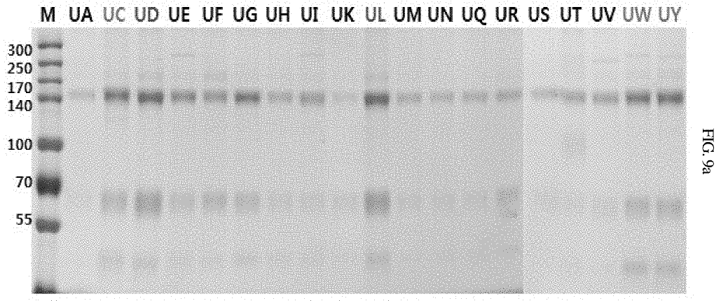

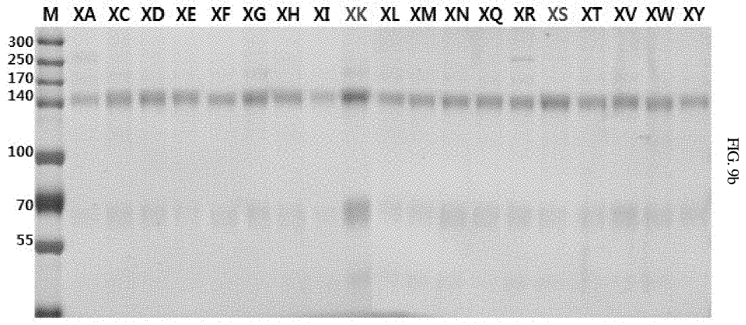

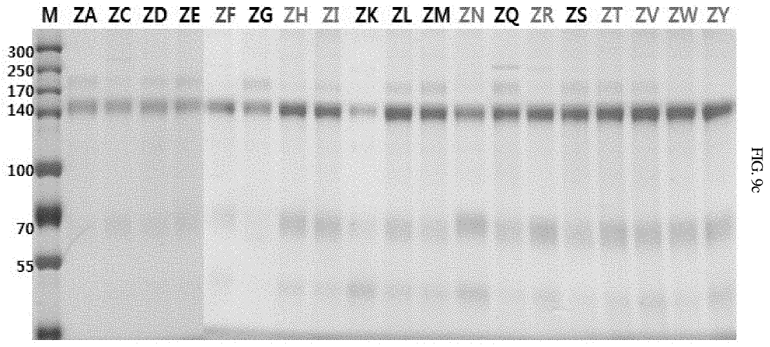

In order to examine whether a better effect was obtained when amino acids corresponding to lock mutation were substituted with other residues, L368, K370, and K409, which were the lock in the double mutation pairs, were substituted with other amino acid residues. Mutation combinations obtained by variously mutating L368, K370, and K409 are summarized in Table 12. The mutation combinations were tested for heterodimerization on SDS-PAGE (NR: 8% SDS-PAGE gel; Sample: 24 ul Loading), and the results are depicted in FIGS. 9a to 9c.

TABLE 12
Lock variants

| | Chain | | | Chain | Chain | | Chain | Chain |
|---|---|---|---|---|---|---|---|---|
| X | A | Chain B | Y | A | B | Z | A | B |
| UA | S364K/ | L368A/ | XA | S364K/ | K370A/ | ZA | S364K/ | K370S/ |
| | K409F | F405R | | K409F | F405R | | K409A | F405R |
| UC | S364K/ | L368C/ | XC | S364K/ | K370C/ | ZC | S364K/ | K370S/ |
| | K409F | F405R | | K409F | F405R | | K409C | F405R |
| UD | S364K/ | L368D/ | XD | S364K/ | K370D/ | ZD | S364K/ | K370S/ |
| | K409F | F405R | | K409F | F405R | | K409D | F405R |
| UE | S364K/ | L368E/ | XE | S364K/ | K370E/ | ZE | S364K/ | K370S/ |
| | K409F | F405R | | K409F | F405R | | K409E | F405R |
| UF | S364K/ | L368F/ | XF | S364K/ | K370F/ | ZF | S364K/ | K370S/ |
| | K409F | F405R | | K409F | F405R | | K409F | F405R |
| UG | S364K/ | L368G/ | XG | S364K/ | K370G/ | ZG | S364K/ | K370S/ |
| | K409F | F405R | | K409F | F405R | | K409G | F405R |
| UH | S364K/ | L368H/ | XH | S364K/ | K370H/ | ZH | S364K/ | K370S/ |
| | K409F | F405R | | K409F | F405R | | K409H | F405R |
| UI | S364K/ | L368I/ | XI | S364K/ | K370I/ | ZI | S364K/ | K370S/ |
| | K409F | F405R | | K409F | F405R | | K409I | F405R |
| UK | S364K/ | L368K/ | XK | S364K/ | K370/ | ZK | S364K/ | K370S/ |
| | K409F | F405R | | K409F | F405R | | K409K | F405R |
| UL | S364K/ | L368/ | XL | S364K/ | K370L/ | ZL | S364K/ | K370S/ |
| | K409F | F405R | | K409F | F405R | | K409L | F405R |
| UM | S364K/ | L368M/ | XM | S364K/ | K370M/ | ZM | S364K/ | K370S/ |
| | K409F | F405R | | K409F | F405R | | K409M | F405R |
| UN | S364K/ | L368N/ | XN | S364K/ | K370N/ | ZN | S364K/ | K370S/ |
| | K409F | F405R | | K409F | F405R | | K409N | F405R |
| UQ | S364K/ | L368Q/ | XQ | S364K/ | K370Q/ | ZQ | S364K/ | K370S/ |
| | K409F | F405R | | K409F | F405R | | K409Q | F405R |
| UR | S364K/ | L368R/ | XR | S364K/ | K370R/ | ZR | S364K/ | K370S/ |
| | K409F | F405R | | K409F | F405R | | K409R | F405R |
| US | S364K/ | L368S/ | XS | S364K/ | K370S/ | ZS | S364K/ | K370S/ |
| | K409F | F405R | | K409F | F405R | | K409S | F405R |
| UT | S364K/ | L368T/ | XT | S364K/ | K370T/ | ZT | S364K/ | K370S/ |
| | K409F | F405R | | K409F | F405R | | K409T | F405R |
| UV | S364K/ | L368V/ | XV | S364K/ | K370V/ | ZV | S364K/ | K370S/ |
| | K409F | F405R | | K409F | F405R | | K409V | F405R |
| UW | S364K/ | L368W/ | XW | S364K/ | K370W/ | ZW | S364K/ | K370S/ |
| | K409F | F405R | | K409F | F405R | | K409W | F405R |
| UY | S364K/ | L368Y/ | XY | S364K/ | K370Y/ | ZY | S364K/ | K370S/ |
| | K409F | F405R | | K409F | F405R | | K409Y | F405R |

The combinations were measured for thermal stability (see Example 2) and the results are given in Table 13.

TABLE 13

| | Chain A | Chain B | Tm |
|---|---|---|---|
| UC | S364K/K409F | L368C/F405R | 63.8 |
| UD | S364K/K409F | L368D/F405R | 60.8 |
| UL | S364K/K409F | L368/F405R | 63.0 |
| UW | S364K/K409F | L368W/F405R | 61.2 |
| UY | S364K/K409F | L368Y/F405R | 61.2 |
| ZF | S364K/K409F | K370S/F405R | 65.6 |
| ZH | S364K/K409H | K370S/F405R | 64.4 |
| ZI | S364K/K409I | K370S/F405R | 62.4 |
| ZN | S364K/K409N | K370S/F405R | 61.0 |
| ZR | S364K/K409R | K370S/F405R | 62.8 |
| ZT | S364K/K409T | K370S/F405R | 65.4 |
| ZV | S364K/K409V | K370S/F405R | 66.0 |
| ZW | S364K/K409W | K370S/F405R | 67.0 |
| ZY | S364K/K409Y | K370S/F405R | 63.8 |

Analysis showed higher thermal stability of 1(409W (tryptophan) than K409F.

Based on the data obtained above, mutations were made of S364K and 1(409W into chain A and K370S and F405R into chain B to produce a double mutation pair, termed AWBB mutation pair, for use in the following Fc heterodimerization test of CH3 domain.

### Example 4: Selection of Mutation for Heavy Chain and Light Chain in Antibody Fab

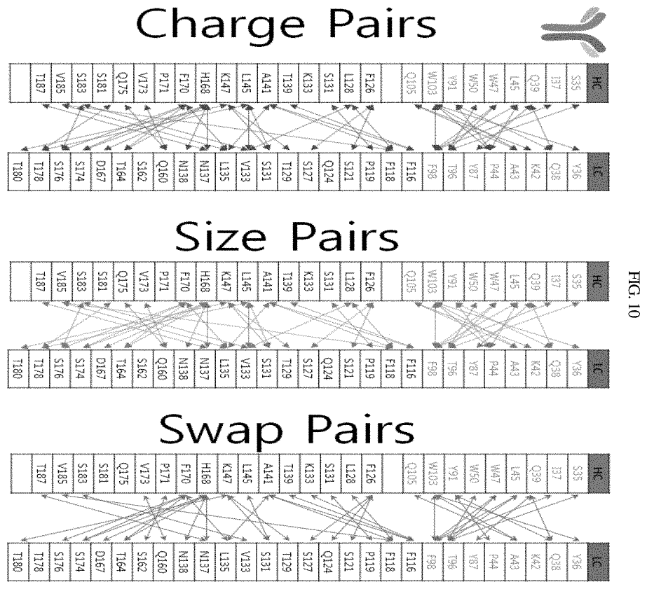

To select mutations in heavy and light chains of antibodies, electrostatic interaction-associated mutations, size-associated mutations, and swapping-associated mutations were carried out. Positions of interaction at heavy and light chains are depicted in FIG. 10.

In order to easily identify mutations in heavy and light chains, antibodies having the same light chain were cloned. 4D9 antibody (anti-Influenza A antibody) and 2B9 antibody (anti-Influenza B antibody), which are both anti-influenza antibodies, have the same light chain (common light chain CLC). Because they have the same light chain, SDS-PAGE analysis allows interaction between heavy and light chains to be easily understood. Although identical in the amino acid sequence of light chain, the two antibodies 4D9 and 2B9 are different from each other with respect to the sequence and size of the heavy chain (the heavy chain of 2B9 has more amino acid residues by six than that of 4D9 and a size (50130.62 Daltons) greater than that of 4D9 (49499.98 Daltons): they can be clearly discriminated on SDS-PAGE). Thus, size analysis on SDS-PAGE makes it possible to understand which of the two chains interacts with the light chain.

Amino acid sequences and coding nucleic acid sequences thereof in the heavy chain variable regions and light chain variable regions of the two antibodies 4D9 and 2B9 are listed in Table 14, below.

TABLE 14

| | Amino Acid Sequence | Nucleic Acid Sequence |
|---|---|---|
| 2B9 Heay | EVQLVESGGGLVQPGKSLRLSC | GAAGTGCAGCTGGTGGAGTCTGGGGGAG |
| Chain | AATGFTFDDYAMHWVRQAPG | GCTTGGTACAGCCTGGCAAGTCCCTGAG |
| Variable | KGLEWVSSLNWKGNSVDYAD | ACTCTCCTGTGCAGCCACTGGATTCACAT |
| Region | SVRGRFTMSRDNAKKLVYLQM | TTGACGATTACGCCATGCACTGGGTCCGC |
| | NGLRGDDTAVYFCAKDNKAD | CAAGCTCCAGGGAAGGGCCTGGAGTGGG |
| | ASMDYYYHHGMDVWGQGTT | TCTCAAGTCTTAATTGGAAGGGAAATAGT |
| | VTVSS (SEQ ID NO: 27) | GTAGACTACGCGGACTCTGTGAGGGGCC |
| | | GATTCACCATGTCCAGAGACAACGCCAA |
| | | GAAACTAGTGTATCTGCAAATGAACGGT |
| | | CTGAGAGGTGACGACACGGCCGTCTATTT |
| | | TTGTGCAAAAGATAATAAAGCGGATGCA |
| | | TCTATGGACTACTACTACCACCACGGTAT |
| | | GGACGTCTGGGGCCAAGGGACCACGGTC |
| | | ACCGTCTCCTCG (SEQ ID NO: 28) |
| 4D9 Heay | QVTLRESGPGLVKPSETLSLTCT | CAGGTCACCTTGAGGGAGTCGGGCCCAG |
| Chain | ISGASINTDYWSWIRQPPGKGLE | GACTGGTGAAGCCTTCGGAGACCCTGTCC |
| Variable | WIGYIYYRGRTNYNPSLRSRVTI | CTCACCTGCACTATCTCCGGTGCCTCCAT |
| Region | SVDTSKNQFSL | CAATACTGACTACTGGAGCTGGATCCGG |
| | QMTSMTAADTAVYYCARDVT | CAGCCCCCAGGGAAGGGACTGGAGTGGA |
| | GISRENAFDIWGQGTLVTVSS | TTGGCTATATCTATTACAGAGGGCGCACC |
| | (SEQ ID NO: 29) | AACTACAACCCCTCCCTCAGGAGCCGAG |
| | | TCACCATATCAGTAGACACGTCCAAGAA |
| | | TCAATTCTCCCTG |
| | | CAGATGACGTCTATGACCGCTGCTGACAC |
| | | GGCCGTATATTACTGTGCGAGAGATGTG |
| | | ACTGGCATCAGTCGAGAAAATGCTTTTGA |
| | | TATCTGGGGCCAAGGCACCCTGGTCACC |
| | | GTCTCCTCG (SEQ ID NO: 30) |
| Light Chain | AIRMTQSPSSLSASVGDRVTTTC | GCCATCCGGATGACCCAGTCTCCATCCTC |
| (CLC) | RASQSISGYLNWYQQKPGKAP | CCTGTCTGCATCTGTAGGAGACAGAGTCA |
| Varuable | KLLIYAASSLQSGVPSRFSGSGS | CCATCACTTGCCGGGCAAGTCAGAGCATT |
| Region | GTDFILTISSLQPEDFATYYCQQ | AGCGGCTATTTAAATTGGTATCAGCAGA |
| | SYSIPTTFGQGTRLEIK (SEQ ID | AACCAGGGAAAGCCCCTAAGCTCTTGAT |
| | NO: 31) | CTATGCTGCATCCAGTTTGCAGAGTGGGG |
| | | TCCCATCAAGGTTCAGTGGCAGTGGATCT |
| | | GGGACAGATTTCACTCTCACCATCAGCAG |
| | | TCTGCAACCTGAAGATTTTGCAACTTACT |
| | | ACTGTCAACAGAGCTACAGTATCCCCACC |
| | | ACCTTCGGCCAAGGGACACGACTGGAGA |
| | | TTAAA (SEQ ID NO: 32) |

The two antibodies 4D9 and 2B9 employ the constant region of IgG1 as a heavy chain constant region and the kappa constant region as a light chain constant region.

Examination was made to see whether a light chain having a mutation introduced thereinto pairs with only a heavy chain having a mutation interacting with the mutation of the light chain. In this regard, a light chain, a heavy chain pairing with the light chain, and a heavy chain forming a mispair with the light chain were co-expressed to afford antibodies. SDS-PAGE analysis of the antibodies in a reducing condition can identify the extent to which the pairings are accurately formed. For convenience, 4D9 heavy chain is referred to as A chain and a light chain pairing therewith as a chain, and 2B9 heavy chain is referred to as B chain and a light chain pairing therewith as b chain (see the following illustrations).

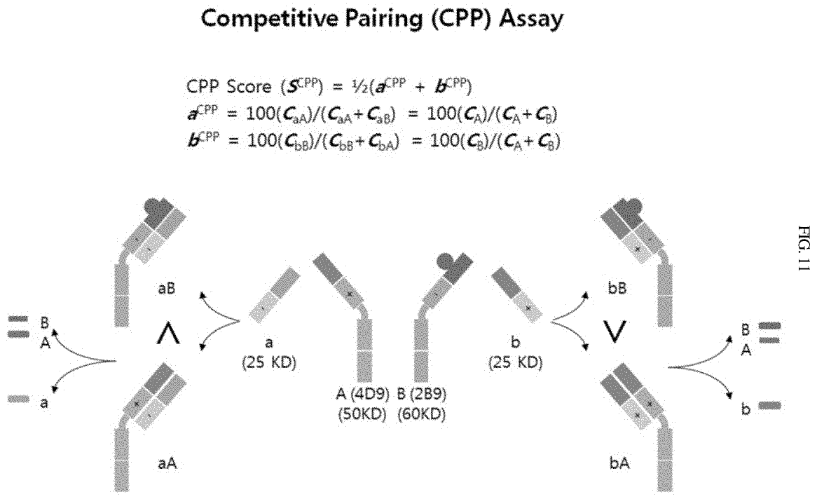

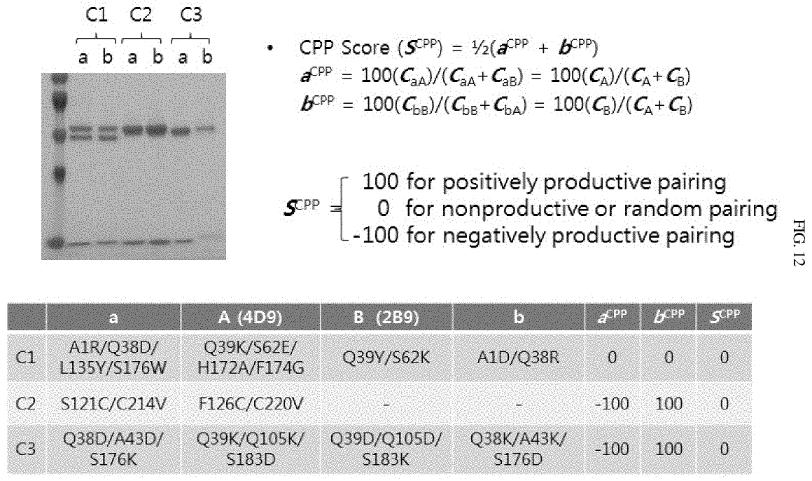

First, 4D9 and 2B9 having conventional mutation pairs between heavy and light chains, already known to be effective, were cloned and co-expressed. Pairing modes established through competition between the resulting mutant chains, that is, two heavy chains and one light chain were examined on SDS-PAGE. (Competitive Pairing (CPP) Assay). The process of performing a competitive pairing (CPP) assay is schematically illustrated in FIG. 11, and the result thus obtained is depicted in FIG. 12. As shown in FIG. 12, all the already known mutation pairs undergo normal pairing and abnormal mispairing, concurrently.

For electrostatic interaction-associated mutations between heavy and light chains, antibodies in which various mutation pairs were introduced with the substitution of corresponding amino acids in B chain (2B9 heavy chain) with K (lysine) and in A chain (4D9 heavy chain) with D (aspartic acid) were subjected to CPP assay on SDS-PAGE (see FIG. 11).

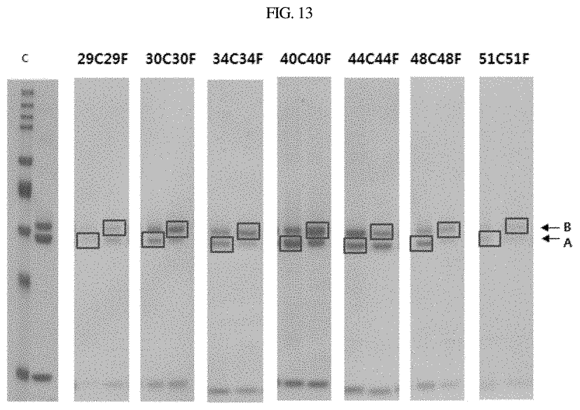

As a result, seven candidate mutation pairs that were identified to undergo relatively accurate pairing were screened in the electrostatic interaction-associated mutation group. CPP assay results on SDS-PAGE of the antibodies into which the seven screened mutation pairs were introduced are given in Table 15 and depicted in FIG. 13.

TABLE 15

| | SOP | | | | | | | |
|---|---|---|---|---|---|---|---|---|
| No | (Symmetric Orthogonal Pairs) | A (4D9) | B (2B9) | a | b | a^{CPP} | b^{CPP} | S^{CPP} |
| c | cΦf | L145 | L145 | S131 | S131 | | | |
| 1 | c29Φf29 | L145D | L145K | S131K | S131D | 58 | 50 | 54 |
| 2 | c30Φf30 | L145D | L145K | V133K | V133D | 50 | 79 | 65 |
| 3 | c34Φf34 | K147D | K147K | T180K | T180D | 49 | 87 | 68 |
| 4 | c40Φf40 | F170D | F170K | L135K | L135D | 59 | 61 | 60 |
| 5 | c44Φf44 | P171D | P171K | S162K | S162D | 59 | 51 | 55 |
| 6 | c48Φf48 | S183D | S183K | V133K | V133D | 62 | 80 | 72 |
| 7 | c51Φf51 | V185D | V185K | L135K | L135D | 67 | 73 | 70 |

(CPP Score (S^{CPP}) = ½(a^{CPP} + b^{CPP})
a^{CPP} = 100(C_{aA})/(C_{aA} + C_{aB}) = 100(C_{A})/(C_{A} + C_{B})
b^{CPP} = 100(C_{bB})/(C_{bB} + C_{bA}) = 100(C_{B})/(C_{A} + C_{B}))

Comparison on SDS-PAGE found some mutation pairs that underwent relatively accurate pairing in the list of mutation pairs using the 4D9 (A chain) and 2B9 (B chain) antibodies (expressed in bold in Table 15). For further study, mutation at position 30 (expressed as c30Φf30) was modified as in Table 16.

TABLE 16
Mutation c30Φf30

| | Heavy Chain | Light Chain | Paring Accuracy |
|---|---|---|---|
| Aa (4D9) | L145D | V133R | 75% |
| Bb (2B9) | L145R | V133D | 60% |

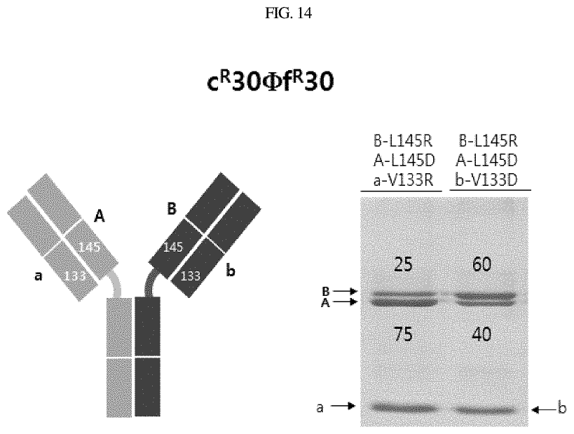

The mutation at position 30 resulted from substitution heavy chain L145 and light chain V133 with K and D, respectively. As is understood from the data of Table 16, the effect (pairing accuracy: Aa pairing or Bb pairing ratio) was observed to be good (Aa pairing accuracy 75%, Bb pairing accuracy: 60%) for the variations in which heavy chain L145 and light chain V133 were substituted with R and D, respectively (see FIG. 14).

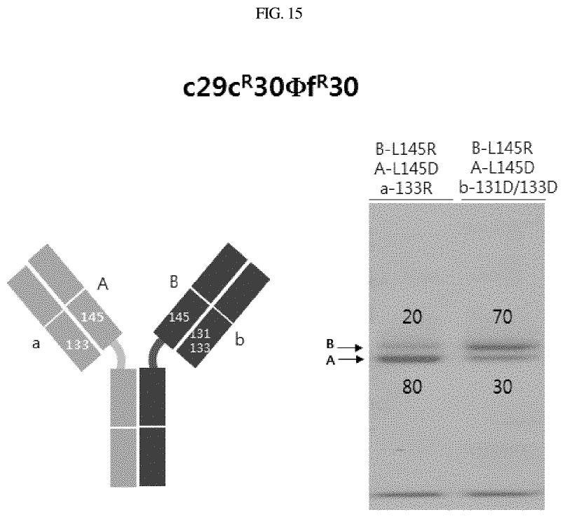

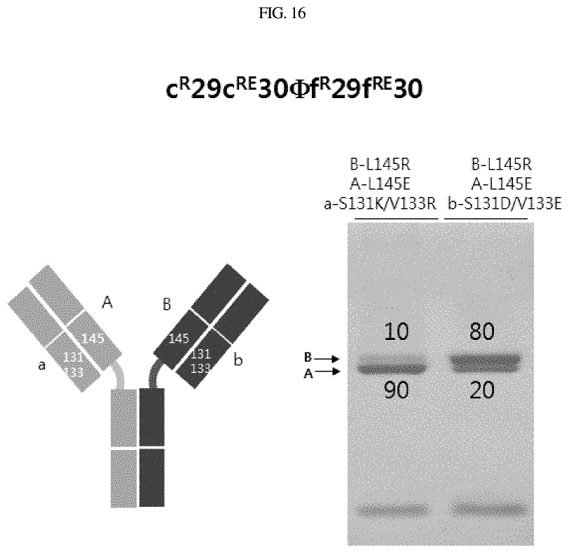

Addition of mutation S131D on the light chain (termed mutation at position 29) to the mutation at position 30 or variations thereof (heavy chain L145 and light chain V133 were substituted with K or R, and D or E, respectively) improved the accuracy of pairing (see Table 17 and FIG. 15 (c29c^{R}30Φf^{R}30 result; Aa pairing accuracy: 80% and Bb pairing accuracy: 70%) and FIG. 16 (c^{R}29c^{RE}30Φf^{R}29f^{RE}30 result; Aa pairing accuracy: 90% and Bb pairing accuracy: 80%)).

TABLE 17

| | | |
|---|---|---|
| Heavy Chain | Light Chain | Paring Accuracy |

c29c^{R}30Φf^{R}30

| | | | |
|---|---|---|---|
| Aa (4D9) | L145D | V133R | 80% |
| Bb (2B9) | L145R | S131D/V133D | 70% |

c^{R}29c^{RE}30Φf^{R}29f^{RE}30

| | | | |
|---|---|---|---|
| Aa (4D9) | L145E | S131K/V133R | 90% |
| Bb (2B9) | L145R | S131D/V133E | 80% |

As can be seen in FIGS. 14 and 15, outstanding pairing accuracy was detected in the cases where heavy chain L145 and light chain V133 were substituted with R and D, respectively and where substitution of light chains S131 and V133 were respectively made by K and R, or D and E as well as in mutation at position 30 wherein heavy chain L145 and light chain V133 were substituted with K and D, respectively.

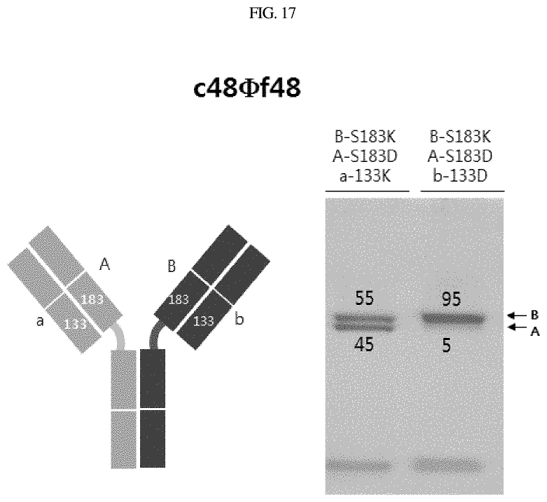

In addition, a mutation pair in which heavy chain S183 and light chain V133 were respectively substituted with K (or R) and D (or E) (termed mutation at position 48) was also observed to be effective (Table 18 and FIG. 17 (Aa pairing accuracy (low bands): 45%, Bb pairing accuracy (upper bands): 95%).

TABLE 18

| LC | V133K (a) | V133D (b) |
|---|---|---|
| 2B9 Heavy Chain (B) | S183K | |
| 4D9 Heavy Chain (A) | S183D | |
| mg/L | 2.6 | 2.4 |

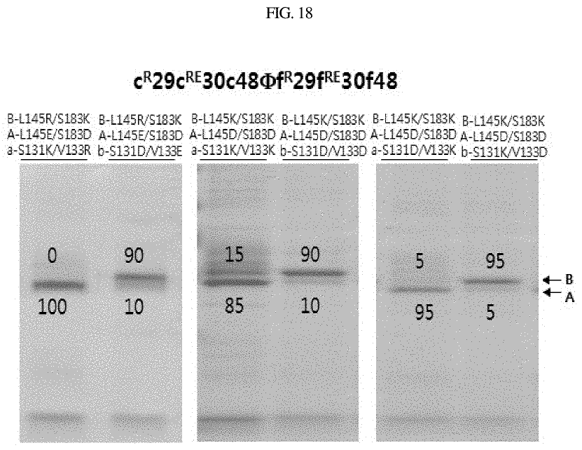

Antibodies containing a combination of the aforementioned mutation at position 29, mutation at position 30, and mutation at position 48 (c29c30c48FΦ29f30f48) or variations thereof were constructed (see Table 19) and then tested for pairing accuracy. The results are depicted in FIG. 18 (lower bands: Aa pairing; and upper bands: Bb paring):

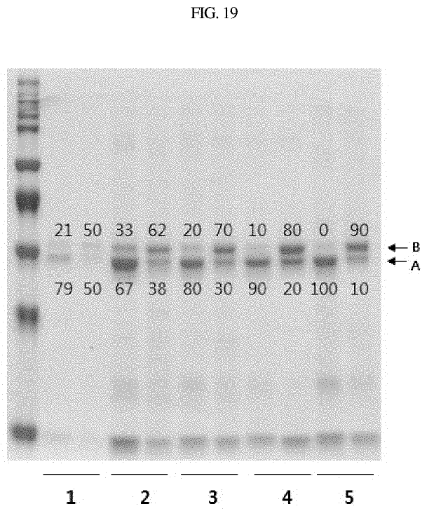

In addition, antibodies containing a variant mutation pair of at least one of mutation at position 29, mutation at position 30, and mutation at position 48 (see Table 20) were examined for pairing accuracy and the results are depicted in FIG. 19 (lower bands: Aa pairing; and upper bands: Bb paring):

TABLE 20

| No. | 1 | | 2 | | 3 | | 4 | | 5 | |
|---|---|---|---|---|---|---|---|---|---|---|
| C | c30Φf30 | | c^{κ}30Φf^{κ}30 | | c29c^{R}30Φf^{R}30 | | c^{R}29c^{RE}30Φ | | c^{R}29c^{RE}30f48Φ | |
| | | | | | | | f^{R}29f^{RE}30 | | f^{R}29f^{RE}30f48 | |
| LC | V133K | V133D | V133R | V133D | V133R | S131D/ | S131K/ | S131D/ | S131K/ | S131D/ |
| | (a) | (b) | | | | V133D | V133R | V133E | V133R | V133E |

| | | | | | |
|---|---|---|---|---|---|
| 4D9 | L145D | L145D | L145D | L145E | L145E/S183D |
| (A) | | | | | |
| 2B9 | L145K | L145R | L145R | L145R | L145R/S183K |
| (B) | | | | | |
| Aa | 79% | 67% | 80% | 90% | 100% |
| Bb | 50% | 62% | 70% | 80% | 90% |

(Aa: Aa pairing accuracy;
Bb: Bb pairing accuracy)

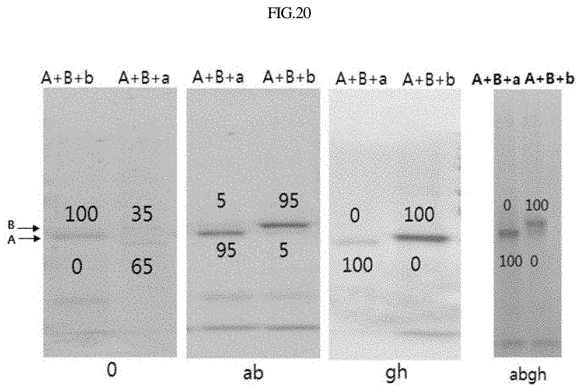

Further, combinations of mutation at position 29, mutation at position 30, and mutation at position 48 were subjected to swapping between D and E to search for combinations that allowed for relative accurate pairing (Table 21 and FIG. 20).

TABLE 21

| Code | A | B | a | b | Aa | Bb |
|---|---|---|---|---|---|---|
| 0 | L145K/S183K | L145D/S183D | S131D/V133D | S131K/V133K | 65% | 100% |
| ab | L145K/S183K | L145E/S183E | S131D/V133D | S131K/V133K | 95% | 95% |
| gh | L145K/S183K | L145D/S183D | S131E/V133E | S131K/V133K | 100% | 100% |
| abgh | L145K/S183K | L145E/S183E | S131E/V133E | S131K/V133K | 100% | 100% |

(Aa: Aa pairing accuracy;
Bb: Bb pairing accuracy)

As can be seen in Table 21 and FIG. 20, all the tested mutation pairs ab, gh, and abgh exhibited a pairing accuracy of 65% or higher, or 95% or higher. Of them, abgh was selected and termed c′29c′30c48Φf′29f′30f′48 mutation pair.

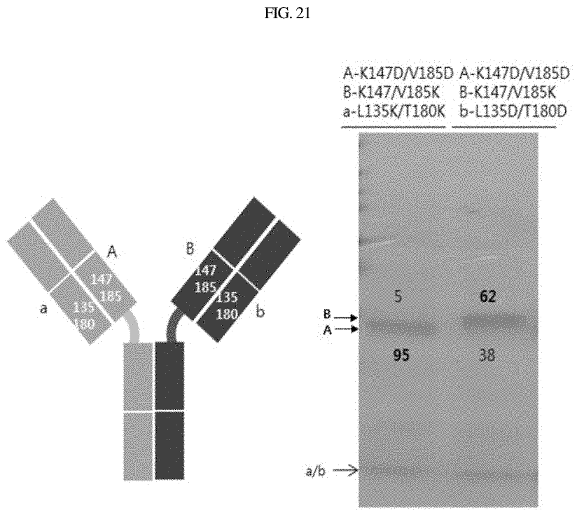

Chains into which combinations of the mutation pairs at position 34 and 51 selected from among the mutation pairs identified in Table 15 were introduced were tested for pairing and the results are given in Table 22 and FIG. 21.

TABLE 22

| | | |
|---|---|---|
| LC | L135K/T180K (a) | L135D/T180D (b) |

| | |
|---|---|
| 4D9 Heavy Chain (A) | K147D/V185D |
| 2B9 Heavy Chain (B) | K147/V185K |
| Aa (%) | 95% |
| Bb (%) | 62% |

In addition, mutation at position 34 and mutation at position 51 of Table 15 were subjected to change from K to R and from D to E, followed by a pairing test to detect combinations having improved pairing accuracy. As a result, a combination in which L135 and

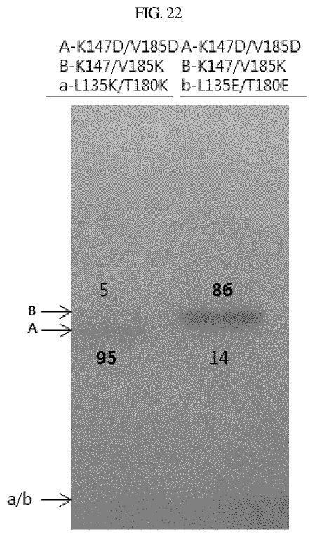

T180 on the light chain were each substituted with E was improved in pairing accuracy and termed c34Φf51 mutation pair (see Table 23 and FIG. 22).

TABLE 23

| | | |
|---|---|---|
| LC | L135K/T180K (a) | L135E/T180E (b) |

| | |
|---|---|
| 4D9 Heavy Chain (A) | K147D/V185D |
| 2B9 Heavy Chain (B) | K147/V185K |
| Aa (%) | 95% |
| Bb (%) | 86% |

Of various mutation pairs found in Table 15, a combination of mutation at position 40 and mutation at position 44 was also obverted to improve in pairing accuracy.

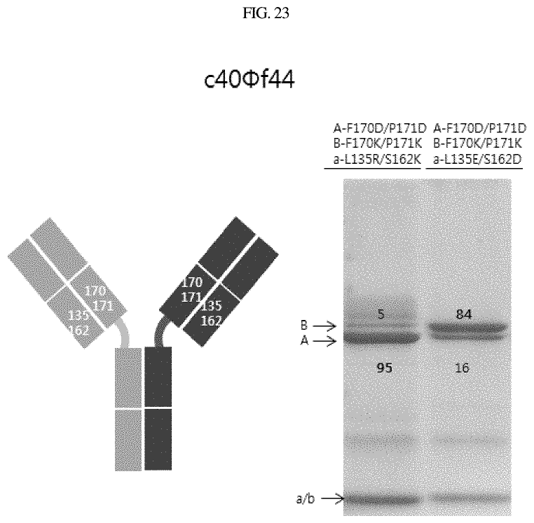

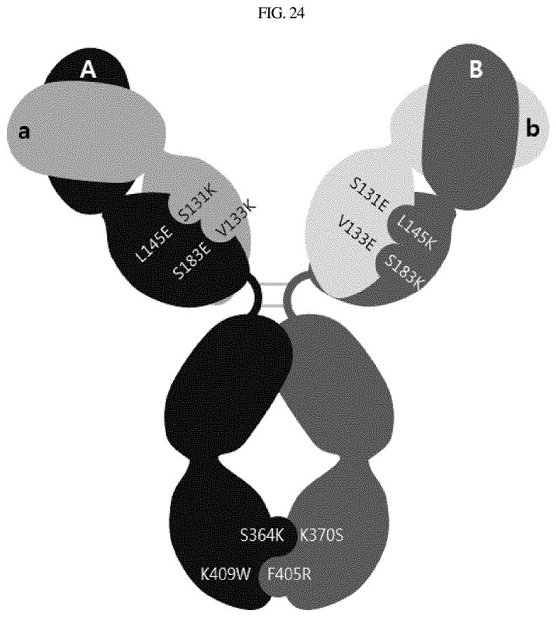

In addition, all the mutation pairs were subjected to swapping between K and R and between D and E to search for a combination that allowed for the most accurate pairing. The combination was a mutation pair in which light chain L135 was substituted with R and E, and was termed c40Φf44 mutation pair (see Table 24 and FIG. 23):

TABLE 24

| | | |
|---|---|---|
| LC | L135R/S162K (a) | L135E/S162D (b) |

| | |
|---|---|
| 4D9 Heavy Chain (A) | F170D/P171D |
| 2B9 Heavy Chain (B) | F170K/P171K |
| Aa (%) | 95% |
| Bb (%) | 84% |

### Example 5: Bispecific Antibody Formation by Coupling Between Heavy Chains and Between Heavy Chain and Light Chain

5.1. Bispecific Antibody Having c′29c′30c48Φf′29f′30f′48 Mutation Pair Introduced Thereto

5.1.1. Test of Coupling Between Heavy and Light Chain Using 4D9/2B9 Antibody

Antibodies were constructed by coupling heavy and light chains containing the c′29c′30c48Φf′29f′30f′48 mutation pair (see Table 25):

TABLE 25

| | Heavy Chain | Light Chain |
|---|---|---|
| | Constant Region | Constant Region |
| Antibody | (HC:CH1) | (LC) |
| 4D9 (Aa) | L145E/S183E | S131K/V133K |
| 2B9 (Bb) | L145K/S183K | S131E/V133E |

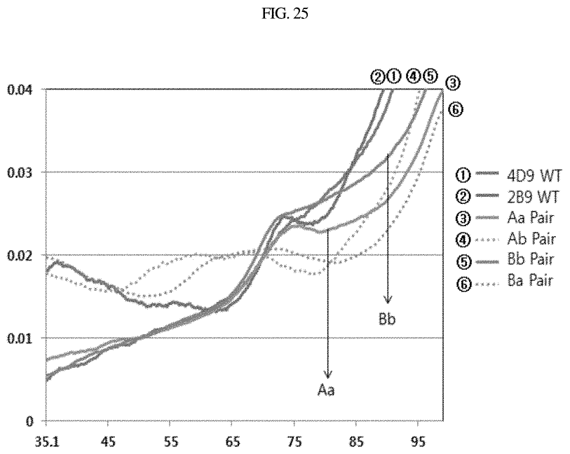

In order to examine the accuracy of pairing among A chain, B chain, a chain, and b chain in the bispecific antibodies, individual heavy chains and light chains were co-expressed in all possible combinations of normal pairs and abnormal mispairs thereamong. Expression levels measured by SDS-PAGE are given in Table 26, below. Thermal stability (Tm) of the combinations was measured and the results are given in Table 27 and FIG. 25:

TABLE 26

| HC | A | | B | |
|---|---|---|---|---|
| LC | a | b | a | b |
| Expression | 89.8 mg/l | 18.8 mg/l | 24.5 mg/l | 68.5 mg/l |
| Amount | | | | |

TABLE 27

| Pairs | Tm |
|---|---|
| 4D9 WT | 69.9 ± 2.00 |
| 2B9 WT | 69.1 ± 1.50 |
| Aa | 70.3 ± 1.70 |
| Ab | 58.5 ± 11.96 |
| Bb | 68.7 ± 0.92 |
| Ba | 58.6 ± 0.53 |

Thermal stability (Tm) was measured with reference to the method explained in Example 2.

As shown in Tables 26 and 27 and FIG. 25, the bispecific antibodies having c′29c′30c48Φf′29f′30f′48 mutation pair introduced thereto were found to be higher in expression level and thermal stability for normal pairing (Aa/Bb) than for abnormal pairing (Ab/Ba).

5.1.2. Trastuzumab/Bevacizumab Bispecific Antibody or Adalimumab/Bevacizumab Bispecific Antibody

Trastuzumab (Herceptin®; Roche), Bevacizumab (Avastin™; Roche), and Adalimumab (Humira®; AbbVie) were purchased and subjected to amino acid sequencing (the Korea Basic Science Institute, Korea). cDNAs corresponding to the amino acid sequences were synthesized and used to construct bispecific antibodies to which the c′29c′30c48Φf′29f′30f′48 mutation pair and the AWBB mutation pair of CH3 domain selected in Example 3 were introduced in the combinations shown in Table 28, below (pcDNA3 vector (see FIG. 26) used).

TABLE 28

| | Heavy Chain | | Light Chain |
|---|---|---|---|
| | Constant Region (HC) | | Constant |
| Antibody | CH1 | CH3 | Region (LC) |

Trabev

| | | | |
|---|---|---|---|
| Trastuzumab (Aa) | L145E/S183E | S364K/K409W | S131K/V133K |
| Bevacizumab (Bb) | L145K/S183K | K370S/F405R | S131E/V133E |

Adabev

| | | | |
|---|---|---|---|
| Adalimumab (Aa) | L145E/S183E | S364K/K409W | S131K/V133K |
| Bevacizumab (Bb) | L145K/S183K | K370S/F405R | S131E/V133E |

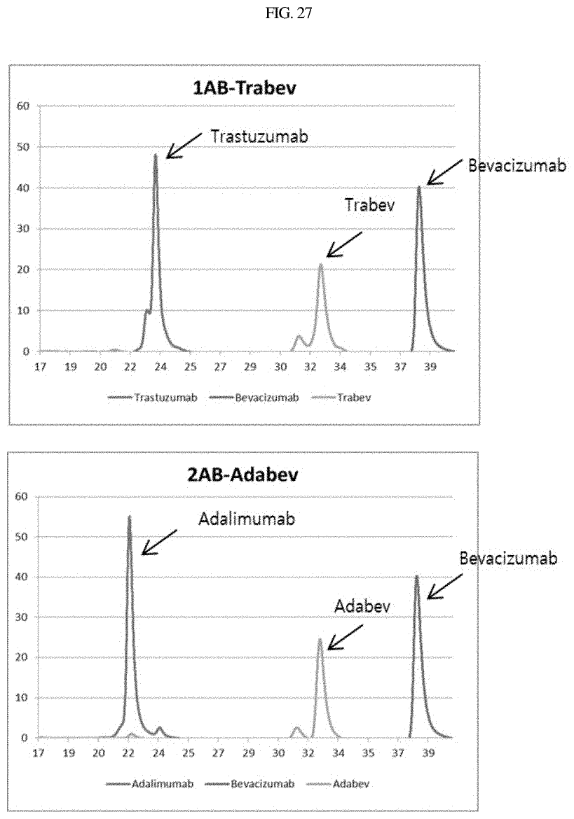

The bispecific antibodies Trabev and Adabev were subjected to hydrophobic interaction chromatography (HIC) in the following condition and the results are depicted in FIG. 27 (y-axis: Value (mAU); and x-axis: time (min)):

Instrument: HPLC-U3000

Column MAbPac Hic-20

Flow rate: 0.2 mL/min

Detection: UV, 280 nm

Mobile Phase: 0.10 M Ammonium acetate, pH 7.0

Passage of the obtained protein through the HIC column formed peaks at different time points depending on the hydrophobicity of the protein, whereby the construction of accurate bispecific antibodies could be accounted for. As can be seen in FIG. 27, peaks for the heterodimerized bispecific antibodies (Trabev and Adabev) are distinctively observed between peaks for two different homodimer antibodies (Trastuzumab and Bevacizumab, or Adalimumab and Bevacizumab), indicating the fine formation of bispecific antibodies, each composed of halves from two different antibodies.

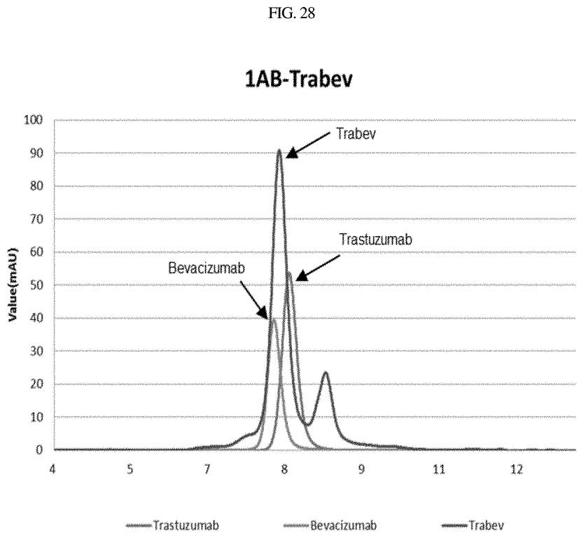

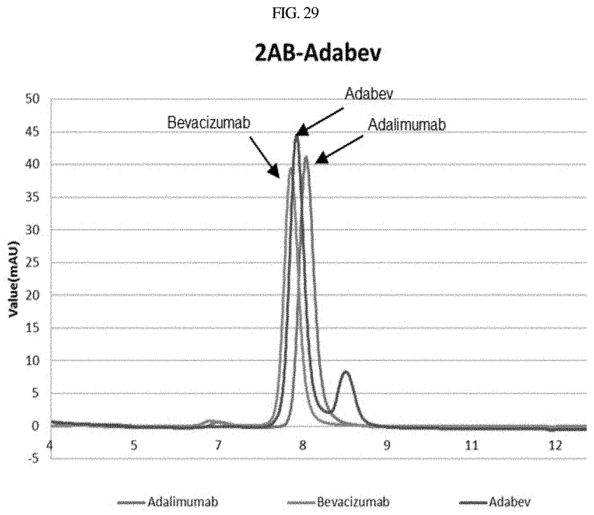

In addition, the bispecific antibodies Trabev and Adabev thus obtained were subjected to size exclusion chromatography (SEC) analysis, and the results are given in Table 29 and FIGS. 28 and 29 (x-axis: time (min)):

Instrument: HPLC-U3000

Column size-exclusion chromatography T SKgel G3000SWXL Tosoh Bioscience

Flow rate: 1.0 mL/min

Detection: UV, 280 nm

Mobile Phase: 25 mM Tris-HCl (pH 8.5), 150 mM NaCl

TABLE 29

| WT (Monospecific Antibody) | | BsAb (Bispecific Antibody) | |
|---|---|---|---|
| 1A. Trastuzumab | 8.007 min. | 1″ Trabev | 7.847 min. |
| 1B. Bevacizumab | 7.743 min. | 2″ Adabev | 7.833 min. |
| 2A. Adalimumab | 7.987 min. | | |

In the SEC analysis, peaks are detected according to protein size and can elucidate protein aggregation. As shown in Table 29 and FIGS. 28 and 29, peaks for the bispecific antibodies are present between peaks of the two corresponding antibodies on the time axis, indicating the fine formation of the bispecific antibodies.

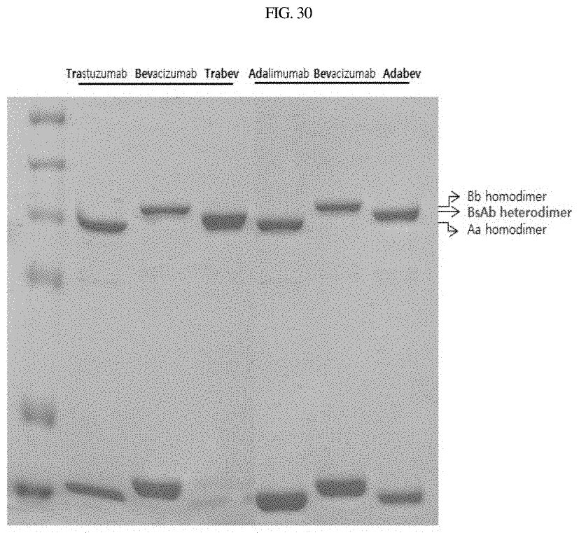

Further, heterodimization modes of the bispecific antibodies Trabev and Adabev on SDS-PAGE are depicted in FIG. 30. As can be seen in FIG. 30, the bispecific antibodies Trabev and Adabev, which are heterodimers, were detected as single bands at intermediate sizes between Trastuzumab and Bevacizumab and between Adalimumab and Bevacizumab, respectively. These results imply that the bispecific antibodies are not homodimers, but are constructed only as a result of normal pairing.

Through ELISA, tests were conducted to examine whether the BsAbs bind effectively to respective antigens (Trastuzumab: Her2), Bevacizumab (VEGF), and Adalimumab (TNF-alpha).

Affinity for antigen was measured as follows:

Reagent

Detection antibody: goat anti-human kappa-HRP (southern biotech, 2060-05)

TMB single solution (LIFE TECHNOLOGY, 002023)

Instrument

Emax precision microplate reader (Molecular devices)

Protocol

Coating buffer: Carbonate buffer pH 9.6

Blocking buffer: protein-free(TBS) blocking buffer (Thermo scientific)

Wash buffer: 0.05% (w/v) Tween20 in TBS, pH7.4 (TBST)

Diluent: 0.05% (w/v) Tween20 in TBS, pH7.4

Stop buffer: 1N Hydrochloric acid solution (HCl)

Protocol

Coating: dilute antigen in coating buffer, load 100 ul of dilution to each well, and incubate at 4□ overnight (Her2, VEGF: 50 ng/well, TNF-alpha: 100 ng/well);

Washing 3 times with washing buffer;

Blocking: load 300 ul of blocking buffer, incubate at room temperature (RT) for 1 hour;

Washing 3 times with washing buffer;

Binding: load antibodies at an aliquot of 100 ng/well, and incubate at RT for 1 hr;

Washing 3 times with washing buffer;

Detection Antibody: dilute goat anti-human kappa-HRP in TBST at a ratio of 1:4000 and incubate at RT for 1 hr;

Washing 3 times with washing buffer

Detection: load 100 ul of TMB solution per well, incubate at RT for 3 min in the dark;

Stop solution: load 100 ul of 1N HCl per well;

Reading: read at optical density 450 nm

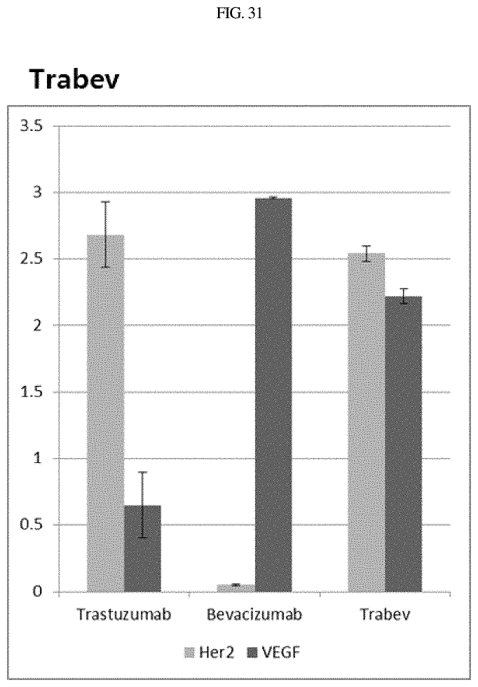

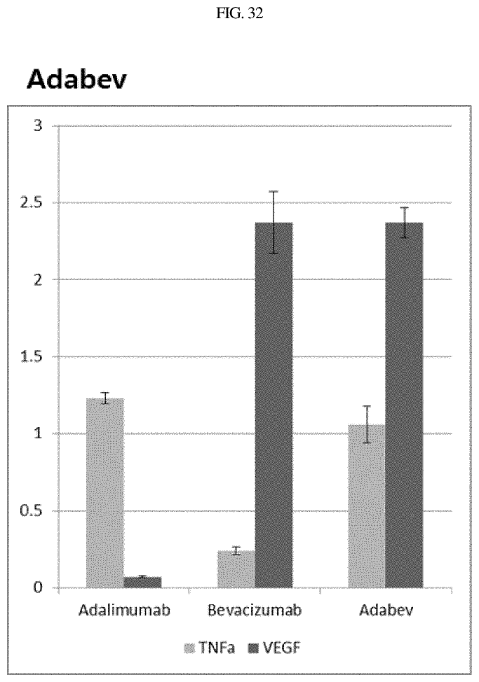

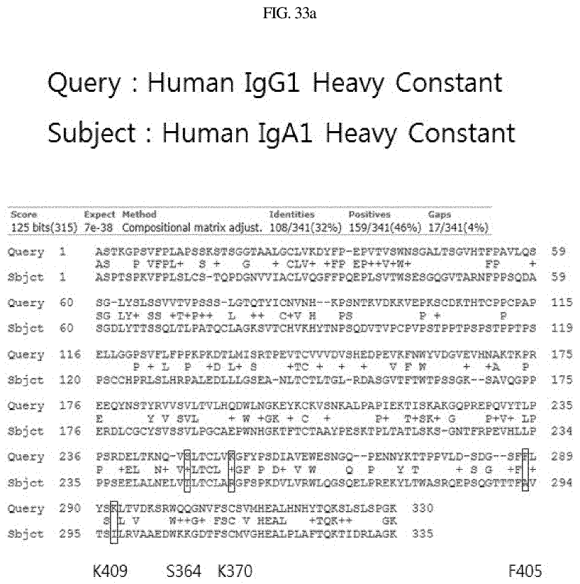

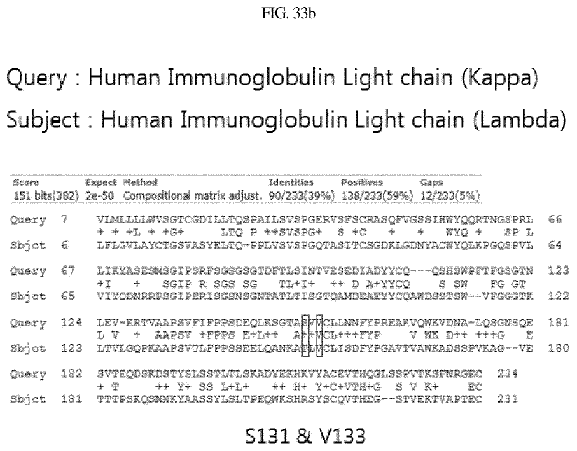

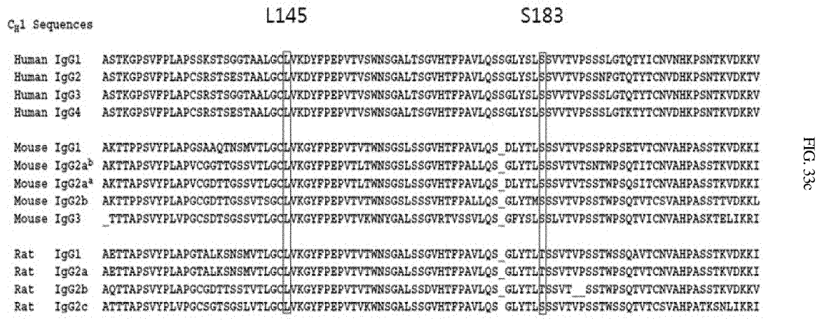

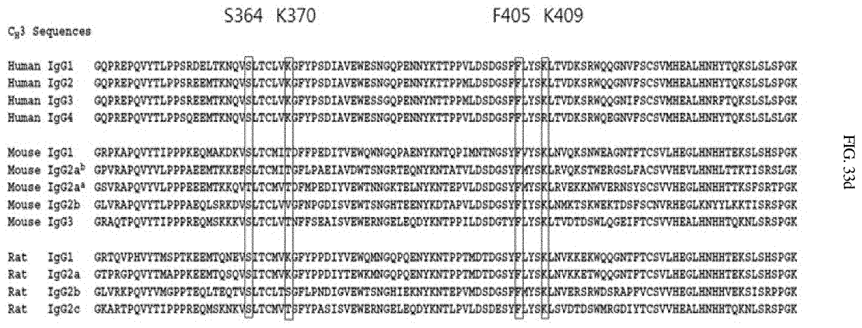

The results thus obtained are given in Table 30 and FIG. 31 (for Trabev) and in Table 31 and FIG. 32 (for Adabev):

TABLE 30

| Antigen | Her2 | | | VEGF | | |
|---|---|---|---|---|---|---|
| Antibody | Trastuzumab | Bevacizumab | Trabev | Trastuzumab | Bevacizumab | Trabev |
| O.D | 2.50 | 0.05 | 2.54 | 0.65 | 2.89 | 2.04 |
| | 2.96 | 0.05 | 2.60 | 0.70 | 2.88 | 2.25 |
| | 2.58 | 0.04 | 2.49 | 0.60 | 3.12 | 2.36 |
| Mean | 2.68 | 0.05 | 2.54 | 0.65 | 2.96 | 2.22 |
| blank | 0.05 | | | 0.05 | | |

TABLE 31

| Antigen | TNF-alpha | | | VEGF | | |
|---|---|---|---|---|---|---|
| Antibody | Adalimumab | Bevacizumab | Adabev | Adalimumab | Bevacizumab | Adabev |
| O.D | 1.24 | 0.23 | 0.93 | 0.07 | 2.24 | 2.37 |
| | 1.26 | 0.22 | 1.16 | 0.06 | 2.27 | 2.28 |
| | 1.19 | 0.27 | 1.09 | 0.07 | 2.60 | 2.47 |
| Mean | 1.23 | 0.24 | 1.06 | 0.07 | 2.37 | 2.37 |
| blank | 0.06 | | | 0.06 | | |

As shown in Table 29 and FIG. 31, the bispecific antibody Trabev was found to bind to both Her2 and VEGF, which are antigens of Trastuzumab and Bevacizumab, respectively. Also, data in Table 30 and FIG. 32 proved that the bispecific antibody Adabev binds to TNF-alpha and VEGF, which are antigens of Adalimumab and Bevacizumab, respectively. These results confirmed that the two bispecific antibodies normally exerting desired functions were successfully constructed.

5.2. Bispecific Antibody Having c34Φf51 Mutation Pair Introduced Thereto

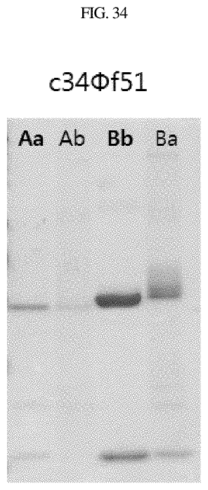

With reference to Example 5.1.1, antibodies having c34Φf51 mutation pair (see Table 23) introduced to 4D9 (Aa) and 2B9 (Bb) thereof were constructed. For each heavy chain, a and b light chains were co-expressed, and inter-light/heavy chain pairing ratios were measured on SDS-PAGE (conducted for heavy chains A and B, each). Tm was measured as in Example 2, and the results are given in Table 32, below and FIG. 34.

TABLE 32

| | Test 1 | | Test 2 | |
|---|---|---|---|---|
| HC | A(K147D/V185D) | | B (K147/V185K) | |
| LC | a | b | b | a |
| | (L135K/T180K) | (L135E/T180E) | (L135E/T180E) | (L135K/T180K) |
| q | 90% | 10% | 89% | 11% |
| Tm | 65.8 | N/A | 65.8 | N/A |

(q: light chain/heavy chain paring ratio (%); N/A: not available)

As shown in Table 32 and FIG. 34, normal heavy chain/light chain pairs (Aa and Bb) were formed at a ratio of as high as 90% and 89%, respectively, and exhibited high thermal stability.

In addition, bispecific antibodies Trabev (Aa: Trastuzumab; Bb: Bevacizumab) and Adabev (Aa: Adalimumab; Bb: Bevacizumab) to each of which the c34Φf51 mutation pair and the CH3 domain AWBB mutation pair (Aa: S364K/K409W; Bb: K370S/F405R) selected in Example 3 were introduced were constructed using Trastuzumab (Herceptin®; Roche), Bevacizumab (Avastin™; Roche), and Adalimumab (Humira®; AbbVie) in a manner similar to the construction procedure for bispecific antibodies of Example 5.1.2.

The constructed bispecific antibodies Trabev and Adabev, each having the c34Φf51 mutation pair and AWBB mutation pair introduced thereinto were analyzed using hydrophobic interaction chromatography (HIC) with reference to the method of Example 5.1.2.

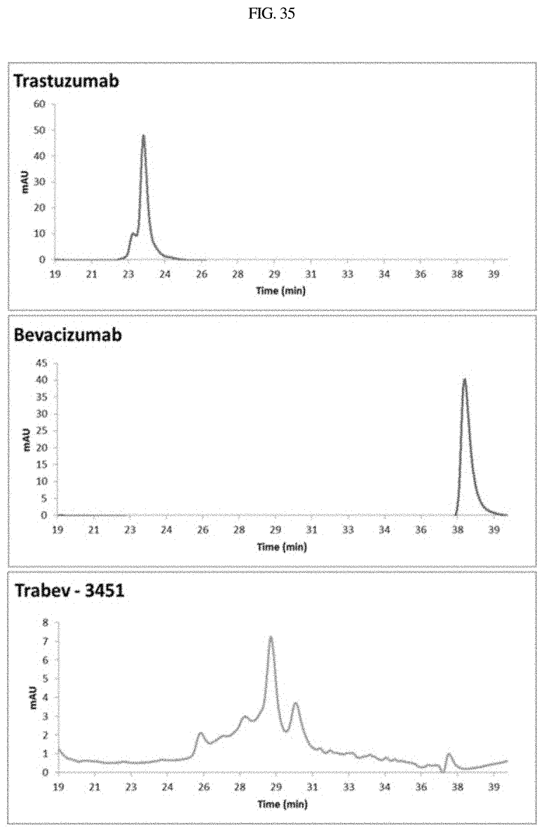

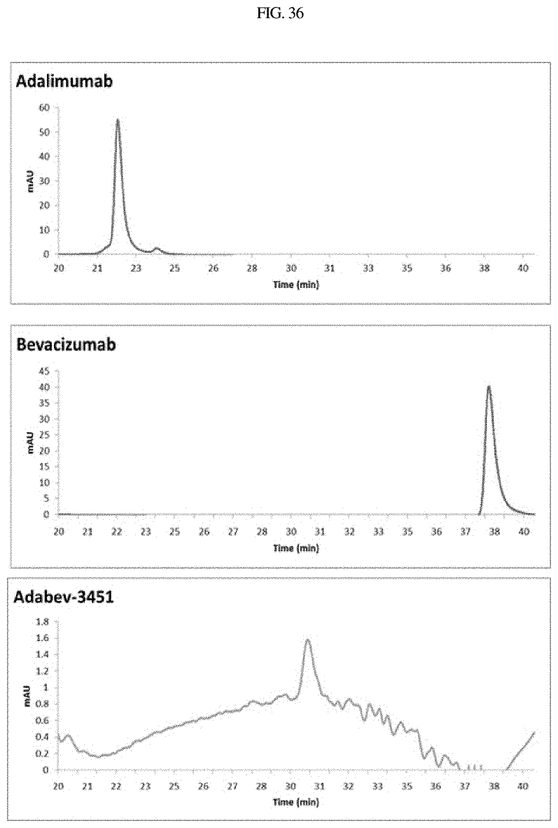

The resulting analysis data are given in Table 33 and FIG. 35 (for Trabev) and in Table 34 and FIG. 36 (for Adabev).

TABLE 33

| # Set 1 | Antibody | Time (min.) |
|---|---|---|
| Aa | Trastuzumab | 23.41 |
| Bb | Bevacizumab | 38.05 |
| 1AB | Trabev | 29.13 |

TABLE 34

| # Set 2 | Antibody | Time (min.) |
|---|---|---|
| Aa | Adalimumab | 22.07 |
| Bb | Bevacizumab | 38.05 |
| 2AB | Adabev | 30.21 |

As can be seen in Table 33 and FIG. 35 and in Table 34 and FIG. 36, the peak of each of the heterodimeric bispecific antibodies (Trabev and Adabev) is distinctively observed between the peaks of two corresponding monospecific antibodies (Trastuzumab and Bevacizumab, or Adalimumab and Bevacizumab). The result implies the fine formation of bispecific antibodies having the c34Φf51 mutation pair and AWBB mutation pair introduced thereto, each composed of halves from two different antibodies.

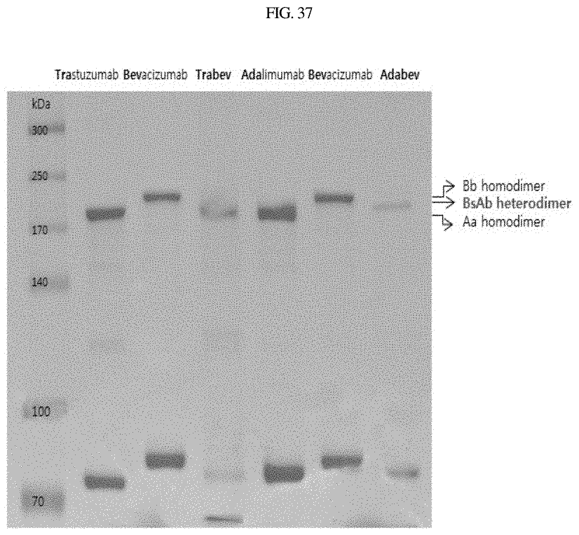

Further, heterodimization modes of the bispecific antibodies having the c34Φf51 mutation pair and AWBB mutation pair introduced thereto are depicted in FIG. 37 (SDS-PAGE gel 6%, Non-Reducing condition). As can be seen in FIG. 37, concurrent introduction of the c34Φf51 mutation pair and AWBB mutation pair into already known antibodies was found to construct pure heterodimers as analyzed by SDS-PAGE.

5.3. Bispecific Antibody Having c40Φf44 Mutation Pair Introduced Thereto

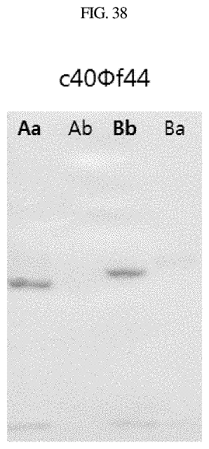

With reference to Example 5.1.1, antibodies having c40Φf44 mutation pair (see Table 24) introduced to 4D9 (Aa) and 2B9 (Bb) thereof were constructed. For each heavy chain, a and b light chains were co-expressed, and inter-light/heavy chain pairing ratios were measured on SDS-PAGE (conducted for heavy chains A and B, each). Tm was measured as in Example 2, and the results are given in Table 35, below and FIG. 38.

TABLE 35

| HC | A (F170D/P171D) | | B (F170K/P171K) | |
|---|---|---|---|---|
| LC | a | b | b | a |
| | (L135R/S162K) | (L135E/S162D) | (L135E/S162D) | (L135R/S162K) |
| q | 99% | 1% | 99% | 1% |
| Tm | 65.0 | N/A | 63.4 | N/A |

(q: light chain/heavy chain pairing ratio (%); N/A: not available)

As shown in Table 35 and FIG. 38, normal heavy chain/light chain pairs (Aa and Bb) were both formed at a ratio of as high as 99%, and exhibited high thermal stability.

In addition, bispecific antibodies Trabev (Aa: Trastuzumab; Bb: Bevacizumab) and Adabev (Aa: Adalimumab; Bb: Bevacizumab) to each of which the c40Φf44 mutation pair and the AWBB mutation pair (A: S364K/K409W; B: K370S/F405R) were introduced were constructed using Trastuzumab (Herceptin®; Roche), Bevacizumab (Avastin™; Roche), and Adalimumab (Humira®; AbbVie) in a manner similar to the construction procedure for bispecific antibodies of Example 5.1.2.

The constructed bispecific antibodies Trabev and Adabev, each having the c40Φf44 mutation pair and AWBB mutation pair introduced thereinto, were analyzed using hydrophobic interaction chromatography (1-11C) with reference to the method of Example 5.1.2.

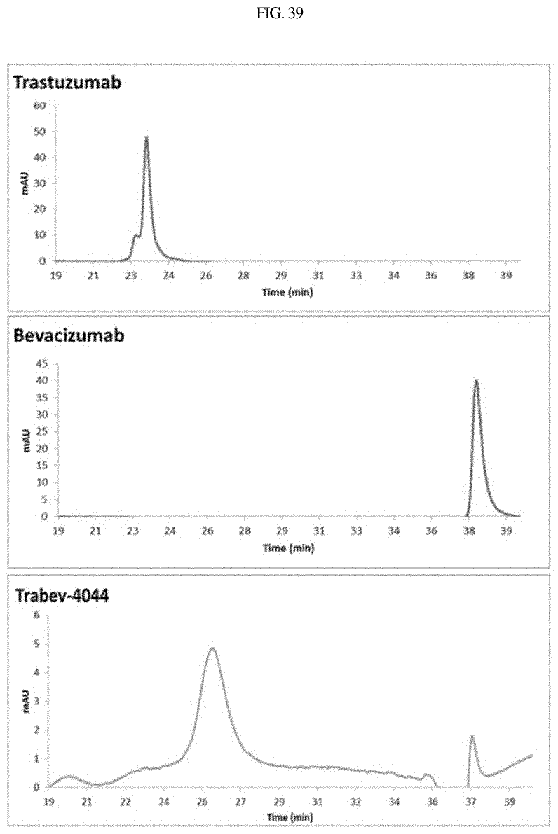

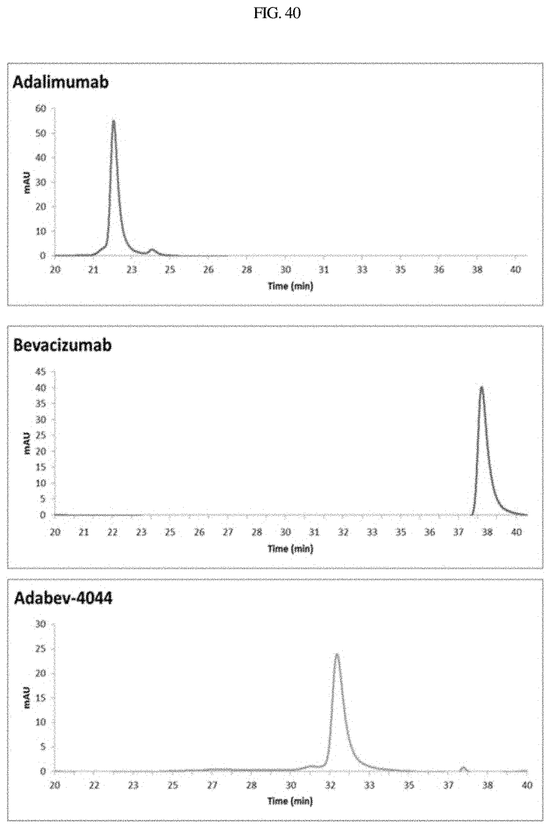

The resulting analysis data are given in Table 36 and FIG. 39 (for Trabev) and in Table 37 and FIG. 40 (for Adabev).

TABLE 36

| # Set 1 | Antibody | Time (min.) |
|---|---|---|
| Aa | Trastuzumab | 23.41 |
| Bb | Bevacizumab | 38.05 |
| 1AB | Trabev | 26.11 |

TABLE 37

| # Set 2 | Antibody | Time (min.) |
|---|---|---|
| Aa | Adalimumab | 22.07 |
| Bb | Bevacizumab | 38.05 |
| 2AB | Adabev | 31.97 |

As can be seen in Table 36 and FIG. 39 and in Table 37 and FIG. 40, the peak of each of the heterodimeric bispecific antibodies (Trabev and Adabev) is distinctively observed between the peaks of two corresponding monospecific antibodies (Trastuzumab and Bevacizumab, or Adalimumab and Bevacizumab). The result implies the fine formation of bispecific antibodies having the c40Φf44 mutation pair and AWBB mutation pair introduced thereto, each composed of halves from two different antibodies. In addition, only one peak distinctively appearing between the two monospecific antibodies indicates that only one kind of normal pairing was made without mispairs between two heavy chains and between heavy and light chains

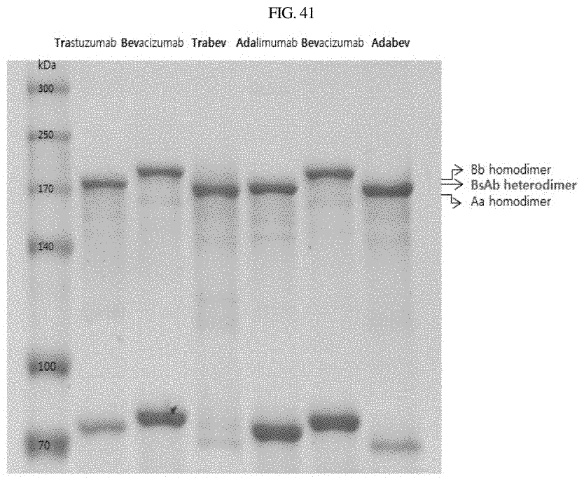

Further, heterodimization modes of the bispecific antibodies having the c40Φf44 mutation pair and AWBB mutation pair introduced thereto are depicted in FIG. 41 (SDS-PAGE gel 6%, Non-Reducing condition). As can be seen in FIG. 41, concurrent introduction of the c40Φf44 mutation pair and AWBB mutation pair into already known antibodies was found to construct pure heterodimers as analyzed by SDS-PAGE.

### Comparative Example 1

Heterodimerization rates were examined for cases where the mutations of CH3 domain proposed herein, which were different from the mutations suggested in Examples 1 to 3 although being identical amino acid pairs, were introduced.

For comparison, TNFRSF1B-Fc fusion protein (Enbrel; Enb) and Fas-Fc fusion protein (Fas) to which mutations of Table 38 were introduced were constructed with reference to Example 1 (expressed as BEAT-A and BEAT-B).

TABLE 38

| | Enb | Fas |
|---|---|---|
| AW/BB | AW | BB |
| | (S364K/K409W) | (K370S/F405R) |
| BEAT-A | S364K/K409W | K370T/F405A |
| BEAT-B | S364T/K409R | K370T/F405S |

TNFRSF1B-Fc fusion protein (A chain) and Fas-Fc fusion protein having CH3 domain mutations selected in Example 3, which are representative of CH3 domain mutations proposed in the description (e.g., A chain having S364K and 1(409W introduced thereto and B chain having K370S and F405R introduced thereto) (expressed as AW/BB in Table 38) were prepared.

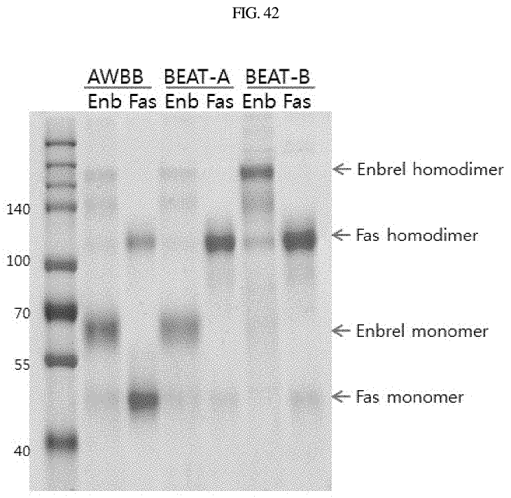

When Enb-Fc and Fas-Fc which had the mutation pairs listed in Table 38 were subjected to single transfection, homodimization modes were observed on SDS-PAGE. The results are given in FIG. 42.

As can be seen in FIG. 42, AW/BB exhibited high percentages of monomers whereas higher percentages of homodimers than monomers were detected in BEAT-A and BEAT-B. On the basis of the result, BEAT-A and BEAT-B are more likely to form homodimers than AW/BB when there is a difference in expression level between A chain and B chain. If a large quantity of homodimers is produced when heavy and light chains are combined so as to construct bispecific antibodies, an accurate heterodimer is difficult to separate from the homodimers because there are almost no differences in physical properties between the heterodimer and the homodimers. Accordingly, minimalizing homodimerization is very important for constructing a bispecific antibody of high purity. A combination of mutations that permits heterodimerization as little as possible is advantageous for the construction of bispecific antibodies.

Taken together, the data show that BEAT-A and BEAT-B have low heterodimerization potentials due to high homodimizerization rates compared to AW/BB and make it difficult to isolate accurate heterodimers.

1. A bispecific protein for targeting two different kinds of targets, the bispecific protein comprising a first CH3 domain or a first Fc region comprising the first CH3 domain and a second CH3 domain or a second Fc region comprising the second CH3 domain, wherein the first CH3 domain and the second CH3 domain are mutated such that at least one selected from among amino acid pairs forming respective amino acid-amino acid bonds between the first CH3 domain and the second CH3 domain is modified by at least one of the following mutations:
(1) a mutation in which the two amino acids in at least one amino acid pair between the CH3 domains are swapped with each other (swapping mutation);
(2) a mutation in which, of at least one amino acid pair between the CH3 domains, one amino acid is substituted with an amino acid having a positive charge while the other is substituted with an amino acid having a negative charge, at least one of the two amino acid residues in the amino acid pair not being hydrophobic (electrostatic interaction-introduced mutation), wherein the amino acid having a negative charge is aspartic acid or glutamic acid and the amino acid having a positive charge is lysine or arginine; and
(3) a mutation in which, of at least one amino acid pair between the CH3 domains, one amino acid is substituted with a large hydrophobic amino acid while the other is substituted with a small hydrophobic amino acid (size mutation), wherein the large hydrophobic amino acid is selected from the group consisting of tryptophan and phenylalanine and the small hydrophobic amino acid is selected from the group consisting of alanine, glycine, and valine,
wherein the first CH3 domain and the second CH3 domain are each independently derived from an immunoglobulin selected from the group consisting of IgG1, IgG2, IgG3, IgG4, IgA1, IgA2, IgE, or IgM.

2. (canceled)

3. (canceled)

4. The bispecific protein of claim 1, wherein the electrostatic interaction-introduced mutation is a mutation in which, of the two amino acids constituting: at least one amino acid pair selected from the group consisting of serine at position 364 and leucine at position 368, threonine at position 394 and threonine at position 394, glutamic acid at position 357 and lysine at position 370, glutamic acid at position 357 and tyrosine at position 349, threonine at position 366 and tyrosine at position 407, and threonine at position 394 and valine at position 397 in an IgG1 CH3 domain (EU numbering); or at least ones amino acid pair at a position corresponding to the at least one amino acid pair in CH3 domains of IgG2, IgG3, IgG4, IgA1, IgA2, IgE, or IgM,
one is substituted with an amino acid having a positive charge and the other is substituted with an amino acid having a negative charge.

5. (canceled)

6. The bispecific protein of claim 4, wherein the electrostatic interaction-introduced mutation comprises at least one of the following mutations on the basis of IgG1 (EU numbering), or at least one of corresponding mutations in the CH3 domains of IgG2, IgG3, IgG4, IgA1, IgA2, IgE, or IgM:
substitution of serine at position 364 with an amino acid having a positive charge and leucine at position 368 with an amino acid having a negative charge;
substitution of threonine at position 394 with an amino acid having a positive charge and threonine at position 394 with an amino acid having a negative charge;
substitution of glutamic acid at position 357 with an amino acid having a positive charge and lysine at position 370 with an amino acid having a negative charge;
substitution of glutamic acid at position 357 with an amino acid having a positive charge and tyrosine at position 349 with an amino acid having a negative charge;
substitution of threonine at position 366 with an amino acid having a positive charge and tyrosine at position 407 with an amino acid having a negative charge;
substitution of threonine at position 394 with an amino acid having a positive charge and valine at position 397 with an amino acid having a negative charge; and
substitution of tyrosine at position 349 with an amino acid having a positive charge and glutamic acid at position 357 with an amino acid having a negative charge.

7. (canceled)

8. The bispecific protein of claim 1, wherein the swapping mutation is substitution in which exchange is made between two paired amino acid residues in:
at least one amino acid pair selected from the group consisting of a pair of serine at position 364 and lysine at position 370, a pair of phenylalanine at position 405 and lysine at position 409, a pair of glutamine at position 347 and lysine at position 360, a pair of glutamic acid at position 357 and tyrosine at position 349, a pair of serine at position 354 and tyrosine at position 349, a pair of glutamic acid at position 357 and lysine at position 370, a pair of lysine at position 360 and tyrosine at position 349, a pair of serine at position 364 and leucine at position 368, a pair of leucine at position 368 and lysine at position 409, a pair of asparagine at position 390 and serine at position 400, a pair of threonine at position 394 and valine at position 397, a pair of leucine at position 398 and lysine at position 392, a pair of tyrosine at position 407 and threonine at position 366, and a pair of threonine at position 411 and lysine at position 370 on the basis of CH3 domain IgG1 (EU numbering); or
at least one amino acid pair at a position corresponding to the at least one amino acid pair in CH3 domains of IgG2, IgG3, IgG4, IgA1, IgA2, IgE, or IgM.

9. The bispecific protein of claim 8, wherein the swapping mutation comprises at least one of the following mutations on the basis of the CH3 domain of IgG1 (EU numbering) or at least one mutation corresponding to the at least one mutation in CH3 domains of IgG2, IgG3, IgG4, IgA1, IgA2, IgE, or 1 IgM:
substitution of serine at position 364 with lysine and lysine at position 370 with serine;
substitution of phenylalanine at position 405 with lysine and lysine at position 409 with phenylalanine;
substitution of tyrosine at position 407 with threonine and threonine at position 366 with tyrosine;
substitution of glutamic acid at position 357 with lysine and lysine at position 370 with glutamic acid; and
substitution of glutamic acid at position 357 with tyrosine and tyrosine at position 349 with serine.

10. The bispecific protein of claim 1, wherein the size mutation comprises substitution in which, of two paired amino acid residues in: at least one amino acid pair selected from the group consisting of a pair of lysine at position 409 and tyrosine at position 407, a pair of lysine at position 409 and phenylalanine at position 405, and a combination thereof on the basis of CH3 domain of IgG1 (EU numbering); or at least one amino acid pair at a position corresponding to the at least one amino acid pair in CH3 domains of IgG2, IgG3, IgG4, IgA1, IgA2, IgE, or IgM,
one is substituted with a large hydrophobic amino acid while the other is substituted with a small hydrophobic amino acid wherein the large hydrophobic amino acid is selected from the group consisting of tryptophan and phenylalanine and the small hydrophobic amino acid is selected from the group consisting of alanine, glycine, and valine.

11. (canceled)

12. The bispecific protein of claim 10, wherein the size mutation comprises: at least one mutation selected from among the following mutations on the basis of the CH3 domain of IgG1 (EU numbering); and at least ones mutation corresponding the at least one mutation in CH3 domains of IgG2, IgG3, IgG4, IgA1, IgA2, IgE, or IgM:
substitution of lysine at position 409 with tryptophan and tyrosine at position 407 with alanine; and
substitution of lysine at position 409 with tryptophan and phenylalanine at position 405 with alanine.

13. (canceled)

14. The bispecific protein of claim 1, comprising:
at least one mutation selected from the group consisting of substitution of one of serine at position 364 and leucine at position 368 with an amino acid having a positive charge and the other with an amino acid having a negative charge, substitution of serine at position 364 with lysine and lysine at position 370 with serine, and substitution of phenylalanine at position 405 with lysine and lysine at position 409 with phenylalanine, on the basis of the CH3 domain of IgG1 (EU numbering); or
at least ones mutation corresponding to the at least one mutation in CH3 domains of IgG2, IgG3, IgG4, IgA1, IgA2, IgE, or IgM.

15. The bispecific protein of claim 14, comprising at least one of the following mutations on the basis of the CH3 of IgG1, or a mutation corresponding to the at least one mutation in CH3 domains of IgG2, IgG3 IgG4 IgA1 IgA2 IgD, IgE, or IgM:
(a) substitution serine at position 364 with lysine or arginine, and leucine at position 368 with aspartic acid or glutamic acid;
(b) substitution of serine at position 364 with lysine, and lysine at position 370 with serine; and
(c) substitution of phenylalanine at position 405 with lysine, and lysine at position 409 with phenylalanine.

16. A bispecific protein for targeting two different kinds of targets, the bispecific protein comprising at least one of the following mutations on the basis of CH3 domain of IgG1 (EU numbering), or a mutation corresponding to the at least one mutation in CH3 domains of IgG2, IgG3, IgG4, IgA1, IgA2, IgE, or IgM:
substitution of lysine at position 409 with phenylalanine or tryptophan, and phenylalanine at position 405 with lysine, arginine, glutamine, or asparagine;
substitution of leucine at position 368 with aspartic acid or glutamic acid, and serine at position 364 with lysine, arginine, or asparagine; and
substitution of lysine at position 370 with serine, and serine at position 364 with lysine, arginine, or asparagine.

17. The bispecific protein of claim 16, comprising at least one of the following mutations on the basis of the CH3 domain of IgG1 (EU numbering), or at least ones mutation corresponding to the at least one mutation in CH3 domains of IgG2, IgG3, IgG4, IgA1, IgA2, IgE, or IgM:
substitution of phenylalanine at position 405 with arginine, and lysine at position 409 with tryptophan;
substitution of serine at position 364 with lysine, and leucine at position 368 with aspartic acid;
substitution of serine at position 364 with lysine, and lysine at position 370 with serine;
substitution of phenylalanine at position 405 with lysine, and lysine at position 409 with phenylalanine;
substitution of phenylalanine at position 405 with arginine, and lysine at position 409 with phenylalanine; and
substitution of phenylalanine at position 405 with lysine, and lysine at position 409 with tryptophan.

18. The bispecific protein of claim 17, comprising the following mutations on the basis of the CH3 domain of IgG1 (EU numbering), or a mutation corresponding to the at least one mutation in CH3 domains of IgG2, IgG3, IgG4, IgA1, IgA2, IgE, or IgM:
substitution of serine at position 364 in the first CH3 domain with lysine and leucine at position 368 in the second CH3 domain with aspartic acid, and substitution of lysine at position 409 in the first CH3 domain with phenylalanine and phenylalanine at position 405 in the second CH3 domain with lysine;
substitution of serine at position 364 in the first CH3 domain with lysine and leucine at position 368 in the second CH3 domain with aspartic acid, and substitution of lysine at position 409 in the first CH3 domain with phenylalanine and phenylalanine at position 405 in the second CH3 domain with arginine;
substitution of serine at position 364 in the first CH3 domain with lysine and lysine at position 370 in the second CH3 domain with serine, and substitution of lysine at position 409 in the first CH3 domain with phenylalanine and phenylalanine at position 405 in the second CH3 domain with lysine;
substitution of serine at position 364 in the first CH3 domain with lysine and lysine at position 370 in the second CH3 domain with serine, and substitution of lysine at position 409 in the first CH3 domain with phenylalanine and phenylalanine at position 405 in the second CH3 domain with arginine;
substitution of serine at position 364 in the first CH3 domain with lysine and leucine at position 368 in the second CH3 domain with aspartic acid, and substitution of lysine at position 409 in the first CH3 domain with tryptophan and phenylalanine at position 405 in the second CH3 domain with lysine;
substitution of serine at position 364 in the first CH3 domain with lysine and leucine at position 368 in the second CH3 domain with aspartic acid, and substitution of lysine at position 409 in the first CH3 domain with tryptophan and phenylalanine at position 405 in the second CH3 domain with arginine;
substitution of serine at position 364 in the first CH3 domain with lysine and lysine at position 370 in the second CH3 domain with serine, and substitution of lysine at position 409 in the first CH3 domain with tryptophan and phenylalanine at position 405 in the second CH3 domain with lysine; or
substitution of serine at position 364 in the first CH3 domain with lysine and lysine at position 370 in the second CH3 domain with serine, and substitution of lysine at position 409 in the first CH3 domain with tryptophan and phenylalanine at position 405 in the second CH3 domain with arginine.

19. (canceled)

20. (canceled)

21. The bispecific protein of claim 1, wherein the bispecific protein is a bispecific antibody or an antigen-binding fragment thereof comprising a first CH1 domain and a first CL (light chain constant region) domain derived respectively from the heavy chain and light chain of an antibody recognizing a first epitope and a second CH1 domain and a second CL domain derived respectively from the heavy chain and light chain of an antibody recognizing a second epitope,
wherein the bispecific antibody or an antigen-binding fragment thereof further comprises at least one of the following mutations on the CH1 domains and the CL domains:
a mutation in which, of the two amino acids constituting each pair of one or more first amino acid pairs selected from among amino acid pairs forming respective amino acid-amino acid bonds between the first CH1 domain and the first CL domain, one is substituted with an amino acid having a positive charge and the other is substituted with an amino acid having a negative charge; and
a mutation in which, of the two amino acids constituting each pair of one or more second amino acid pairs selected from among amino acid pairs forming respective amino acid-amino acid bonds between the second CH1 domain and the second CL domain, one is substituted with an amino acid having a positive charge and the other is substituted with an amino acid having a negative charge, and
wherein the amino acid having a positive charge is lysine or arginine, the amino acid having a negative charge is aspartic acid or glutamic acid.

22. The bispecific protein of claim 21, wherein:
the amino acids substituted respectively in the first CH1 domain and the second CH1 domain have opposite charges,
the amino acids substituted respectively in the first CL domain and the first CH1 domain have opposite charges, and
the amino acids substituted respectively in the second CL domain and the second CH1 domain have opposite charges.

23. The bispecific protein of claim 21, wherein the amino acid to be substituted with an amino acid having a positive or negative charge in the CH1 domain is at least one of the following amino acids on the basis of the CH1 domain of IgG1 (EU numbering), or an amino acid at a position corresponding to the at least one amino acid in CH1 domains of IgG2, IgG3, IgG4, IgA1, IgA2, IgE, or IgM:
leucine at position 145,
serine at position 183,
lysine at position 147,
phenylalanine at position 170,
proline at position 171, and
valine at position 185, and
the amino acid to be substituted with an amino acid having a positive or negative charge in the CL domain is at least one of the following amino acids on the basis of the CL domain of kappa type (EU numbering), or an amino acid at a position corresponding to the at least one amino acid in the CL domain of lambda type:
serine at position 131,
valine at position 133,
leucine at position 135,
serine at position 162, and
threonine at position 180.

24. The bispecific protein of claim 21, wherein
a set of two amino acids forming an amino acid pair between the CH1 domain and the CL domain is at least one of the following amino acid pairs on the basis of the CH1 domain of IgG1 and the CL domain of kappa type (EU numbering), or at least one amino acid pair at a position corresponding to the at least one amino acid pair in CH1 domains and CL domain of lambda type of IgG2, IgG3, IgG4, IgA1, IgA2, IgE, or IgM:
a pair of leucine at position 145 in the CH1 domain and serine at position 131 in the CL domain,
a pair of leucine at position 145 in the CH1 domain and valine at position 133 in the CL domain,
a pair of serine at position 183 in the CH1 domain and valine at position 133 in the CL domain,
a pair of lysine at position 147 in the CH1 domain and threonine at position 180 in the CL domain,
a pair of valine at position 185 in the CH1 domain and leucine at position 135 in the CL domain,
a pair of phenylalanine at position 170 in the CH1 domain and leucine at position 135 in the CL domain, and
a pair of proline at position 171 in the CH1 domain and serine at position 162 in the CL domain.

25. The bispecific protein of claim 16, wherein the bispecific protein is a bispecific antibody or an antigen-binding fragment thereof comprising a first CH1 domain and a first CL (light chain constant region) domain derived respectively from the heavy chain and light chain of an antibody recognizing a first epitope and a second CH1 domain and a second CL domain derived respectively from the heavy chain and light chain of an antibody recognizing a second epitope,
wherein the bispecific antibody or an antigen-binding fragment thereof further comprises at least one of the following mutations on the CH1 domains and the CL domains:
a mutation in which, of the two amino acids constituting each pair of one or more first amino acid pairs selected from among amino acid pairs forming respective amino acid-amino acid bonds between the first CH1 domain and the first CL domain, one is substituted with an amino acid having a positive charge and the other is substituted with an amino acid having a negative charge; and
a mutation in which, of the two amino acids constituting each pair of one or more second amino acid pairs selected from among amino acid pairs forming respective amino acid-amino acid bonds between the second CH1 domain and the second CL domain, one is substituted with an amino acid having a positive charge and the other is substituted with an amino acid having a negative charge, and
wherein the amino acid having a positive charge is lysine or arginine, the amino acid having a negative charge is aspartic acid or glutamic acid.

26. The bispecific protein of claim 25, wherein:
the amino acids substituted respectively in the first CH1 domain and the second CH1 domain have opposite charges,
the amino acids substituted respectively in the first CL domain and the first CH1 domain have opposite charges, and
the amino acids substituted respectively in the second CL domain and the second CH1 domain have opposite charges.

27. The bispecific protein of claim 25, wherein the amino acid to be substituted with an amino acid having a positive or negative charge in the CH1 domain is at least one of the following amino acids on the basis of the CH1 domain of IgG1 (EU numbering), or at least one amino acid at a position corresponding to the at least one amino acid in CH1 domains of IgG2, IgG3, IgG4, IgA1, IgA2, IgE, or IgM:
leucine at position 145,
serine at position 183,
lysine at position 147,
phenylalanine at position 170,
proline at position 171, and
valine at position 185, and
the amino acid to be substituted with an amino acid having a positive or negative charge in the CL domain is at least one of the following amino acids on the basis of the CL domain of kappa type (EU numbering), or an amino acid at a position corresponding to the at least one amino acid in the CL domain of lambda type:
serine at position 131,
valine at position 133,
leucine at position 135,
serine at position 162, and
threonine at position 180.

28. The bispecific protein of claim 25, wherein
a set of two amino acids forming an amino acid pair between the CH1 domain and the CL domain is at least one of the following amino acid pairs on the basis of the CH1 domain of IgG1 and the CL domain of kappa type (EU numbering), or at least one amino acid pair at a position corresponding to the at least one amino acid pair in CH1 domains and CL domain of lambda type of IgG2, IgG3, IgG4, IgA1, IgA2, IgE, or IgM:
a pair of leucine at position 145 in the CH1 domain and serine at position 131 in the CL domain,
a pair of leucine at position 145 in the CH1 domain and valine at position 133 in the CL domain,
a pair of serine at position 183 in the CH1 domain and valine at position 133 in the CL domain,
a pair of lysine at position 147 in the CH1 domain and threonine at position 180 in the CL domain,
a pair of valine at position 185 in the CH1 domain and leucine at position 135 in the CL domain,
a pair of phenylalanine at position 170 in the CH1 domain and leucine at position 135 in the CL domain, and
a pair of proline at position 171 in the CH1 domain and serine at position 162 in the CL domain.

29-42. (canceled)

43. A method for constructing a bispecific protein for targeting different targets, the method comprising one of the following mutation introducing steps to introduce at least one mutation into at least one selected from amino acid pairs forming amino acid-amino acid bonds between a first CH3 domain and a second CH3 domain:
swapping the two amino acids in at least one amino acid pair selected from amino acid pairs forming amino-amino acid bonds between a first CH3 domain and a second CH3 domain with each other;
substituting one of the two amino acids in at least one amino acid pair selected from amino acid pairs forming amino-amino acid bonds between the CH3 domains with an amino acid having a positive charge and the other with an amino acid having a negative charge, wherein the amino acid having a negative charge is aspartic acid or glutamic acid and the amino acid having a positive charge is lysine or arginine; and
substituting one of the two amino acids in at least one amino acid pair selected from amino acid pairs forming amino-amino acid bonds between the CH3 domains with a large hydrophobic amino acid and the other with a small hydrophobic amino acid, wherein the large hydrophobic amino acid is selected from the group consisting of tryptophan and phenylalanine and the small hydrophobic amino acid is selected from the group consisting of alanine, glycine, and valine.

44. The method of claim 43, further comprising the following CH1 and CL domain mutating steps of:
substituting one of the two amino acid residues constituting at least one selected from amino acid pairs forming amino acid-amino acid bonds between a first CH1 domain derived from the heavy chain and a first CL domain of an antibody recognizing a first epitope with an amino acid having a positive charge and the other with an amino acid having a negative charge; and
substituting one of the two amino acid residues constituting at least one selected from amino acid pairs forming amino acid-amino acid bonds between a second CH1 domain derived from the heavy chain and a second CL domain of an antibody recognizing a second epitope with an amino acid having a positive charge and the other with an amino acid having a negative charge.

45-48. (canceled)

49. The bispecific protein of claim 21, wherein
the antibody recognizing a first epitope is an anti-influenza B antibody comprising the heavy chain variable region of SEQ ID NO: 27 and the light chain variable region of SEQ ID NO: 31; and
the antibody recognizing a second epitope is an anti-influenza A antibody comprising the heavy chain variable region of SEQ ID NO: 29 and the light chain variable region of SEQ ID NO: 31.

50. (canceled)


SEQUENCE LISTING (54 sequences)

SEQ ID NO: 1 - protein, 108 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 2 - protein, 105 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVE

SEQ ID NO: 3 - protein, 110 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHT

SEQ ID NO: 4 - protein, 110 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVSPNMVPHAHHAQ

SEQ ID NO: 5 - protein, 108 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPP

SEQ ID NO: 6 - protein, 108 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPP

SEQ ID NO: 7 - protein, 110 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASS

SEQ ID NO: 8 - protein, 110 aa, Homo sapiens
ASTQSPSVFPLTRCCKNIPSNATSVTLGCLATGYFPEPVMVTCDTGSLNGTTMTLPATTLTLSGHYATISLLTVSGAWAKQMFTCRVAHTPSSTDWVDNKTFSVCSRDFT

SEQ ID NO: 9 - protein, 110 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELP

SEQ ID NO: 10 - protein, 105 aa, Homo sapiens [sequence omitted]

SEQ ID NO: 11 - protein, 103 aa, Homo sapiens
KANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 12 - protein, 103 aa, Homo sapiens
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 103 aa, Homo sapiens [sequence omitted]

SEQ ID NO: 14 - protein, 103 aa, Homo sapiens
KAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 15 - protein, 108 aa, Homo sapiens
KGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 108 aa, Homo sapiens
KGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 108 aa, Homo sapiens
KGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 108 aa, Homo sapiens
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 120 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVS

SEQ ID NO: 20 - protein, 120 aa, Homo sapiens
GNTFRPEVHLLPPPSEELALNELVTLTCLARGFSPKDVLVRWLQGSQELPQEPSQGTTTFAVTSILRVAAEDWKKGDTFSCMVGHEALPLAFTQKTIDRLAFTQKTIDRLAGKPTHVNVS

SEQ ID NO: 21 - protein, 119 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMKVK

SEQ ID NO: 22 - protein, 120 aa, Homo sapiens
AAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKELDVCVEEAEGEA

SEQ ID NO: 23 - protein, 120 aa, Homo sapiens
KGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVS

SEQ ID NO: 24 - DNA, 1386 bp, Artificial Sequence
  Synthetic_CDS of TNFRSF1B (Enbrel  NM_001066), wherein 1-771 region encodes ectodomain
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 25 - DNA, 1008 bp, Artificial Sequence
  Synthetic_CDS of Fas Receptor (NM_000043), wherein 1-519 region encodes ectodomain
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 26 - DNA, 684 bp, Artificial Sequence
  Synthetic_CDS of Human Fc (from Human Immunoglobulin G)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 27 - protein, 127 aa, Artificial Sequence
  Synthetic_Heavy Chain Variable Region of anti-Influenza B antibody, 2B9
EVQLVESGGGLVQPGKSLRLSCAATGFTFDDYAMHWVRQAPGKGLEWVSSLNWKGNSVDYADSVRGRFTMSRDNAKKLVYLQMNGLRGDDTAVYFCAKDNKADASMDYYYHHGMDVWGQGTTVTVSS

SEQ ID NO: 28 - DNA, 381 bp, Artificial Sequence
  Synthetic_Nucleic Acid Sequence Encoding Heavy Chain Variable Region of 2B9
gaagtgcagctggtggagtctgggggaggcttggtacagcctggcaagtccctgagactctcctgtgcagccactggattcacatttgacgattacgccatgcactgggtccgccaagctccagggaagggcctggagtgggtctcaagtcttaattggaagggaaatagtgtagactacgcggactctgtgaggggccgattcaccatgtccagagacaacgccaagaaactagtgtatctgcaaatgaacggtctgagaggtgacgacacggccgtctatttttgtgcaaaagataataaagcggatgcatctatggactactactaccaccacggtatggacgtctggggccaagggaccacggtcaccgtctcctcg

SEQ ID NO: 29 - protein, 121 aa, Artificial Sequence
  Synthetic_Heavy Chain Variable Region of anti-Influenza A antibody, 4D9
QVTLRESGPGLVKPSETLSLTCTISGASINTDYWSWIRQPPGKGLEWIGYIYYRGRTNYNPSLRSRVTISVDTSKNQFSLQMTSMTAADTAVYYCARDVTGISRENAFDIWGQGTLVTVSS

SEQ ID NO: 30 - DNA, 363 bp, Artificial Sequence
  Synthetic_Nucleic Acid Sequence Encoding Heavy Chain Variable Region of anti-Influenza A antibody, 4D9
caggtcaccttgagggagtcgggcccaggactggtgaagccttcggagaccctgtccctcacctgcactatctccggtgcctccatcaatactgactactggagctggatccggcagcccccagggaagggactggagtggattggctatatctattacagagggcgcaccaactacaacccctccctcaggagccgagtcaccatatcagtagacacgtccaagaatcaattctccctgcagatgacgtctatgaccgctgctgacacggccgtatattactgtgcgagagatgtgactggcatcagtcgagaaaatgcttttgatatctggggccaaggcaccctggtcaccgtctcctcg

SEQ ID NO: 31 - protein, 107 aa, Artificial Sequence
  Synthetic_Common Light Chain for 4D9 and 2B9
AIRMTQSPSSLSASVGDRVTITCRASQSISGYLNWYQQKPGKAPKLLIYAASSLQSGVPSRFSGSGSGTDFTLTISSLQPEDFATYYCQQSYSIPTTFGQGTRLEIK

SEQ ID NO: 32 - DNA, 321 bp, Artificial Sequence
  Synthetic_Nucleic Acid Sequence Encoding Common Light Chain for 4D9 and 2B9
gccatccggatgacccagtctccatcctccctgtctgcatctgtaggagacagagtcaccatcacttgccgggcaagtcagagcattagcggctatttaaattggtatcagcagaaaccagggaaagcccctaagctcttgatctatgctgcatccagtttgcagagtggggtcccatcaaggttcagtggcagtggatctgggacagatttcactctcaccatcagcagtctgcaacctgaagattttgcaacttactactgtcaacagagctacagtatccccaccaccttcggccaagggacacgactggagattaaa

SEQ ID NO: 33 - protein, 330 aa, Artificial Sequence [sequence omitted]

SEQ ID NO: 34 - protein, 335 aa, Artificial Sequence
  Synthetic_Human IgA1 Heavy Constant Region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 35 - protein, 228 aa, Artificial Sequence
  Synthetic_Human Immunoglobulin Light chain (Kappa)
VLMLLLLWVSGTCGDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 226 aa, Artificial Sequence
  Synthetic_Human Immunoglobulin Light chain (Lambda)
LFLGVLAYCTGSVASYELTQPPLVSVSPGQTASITCSGDKLGDNYACWYQLKPGQSPVLVIYQDNRRPSGIPERISGSNSGNTATLTISGTQAMDEAEYYCQAWDSSTSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 37 - protein, 97 aa, Artificial Sequence
  Synthetic_CH1 domain of mouse IgG1
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKI

SEQ ID NO: 38 - protein, 97 aa, Artificial Sequence
  Synthetic_CH1 domain of mouse IgG2ab
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKI

SEQ ID NO: 39 - protein, 97 aa, Artificial Sequence
  Synthetic_CH1 domain of mouse IgG2aa
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 40 - protein, 97 aa, Artificial Sequence
  Synthetic_CH1 domain of mouse IgG2b
AKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKL

SEQ ID NO: 41 - protein, 96 aa, Artificial Sequence
  Synthetic_CH1 domain of mouse IgG3
TTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRI

SEQ ID NO: 42 - protein, 97 aa, Artificial Sequence
  Synthetic_CH1 domain of rat IgG1
AETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKI

SEQ ID NO: 43 - protein, 97 aa, Artificial Sequence
  Synthetic_CH1 domain of rat IgG2a
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKI

SEQ ID NO: 44 - protein, 95 aa, Artificial Sequence
  Synthetic_CH1 domain of rat IgG2b
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKV

SEQ ID NO: 45 - protein, 97 aa, Artificial Sequence
  Synthetic_CH1 domain of rat IgG2c
ATTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRI

SEQ ID NO: 46 - protein, 107 aa, Artificial Sequence
  Synthetic_CH3 domain of mouse IgG1
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 47 - protein, 107 aa, Artificial Sequence
  Synthetic_CH3 domain of mouse IgG2ab
GPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 48 - protein, 107 aa, Artificial Sequence
  Synthetic_CH3 domain of mouse IgG2aa
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 49 - protein, 107 aa, Artificial Sequence
  Synthetic_CH3 domain of mouse IgG2b
GLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 50 - protein, 107 aa, Artificial Sequence
  Synthetic_CH3 domain of mouse IgG3
GRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 51 - protein, 107 aa, Artificial Sequence
  Synthetic_CH3 domain of rat IgG1
GRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 52 - protein, 107 aa, Artificial Sequence
  Synthetic_CH3 domain of rat IgG2a
GTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 53 - protein, 107 aa, Artificial Sequence
  Synthetic_CH3 domain of rat IgG2b
GLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 54 - protein, 107 aa, Artificial Sequence
  Synthetic_CH3 domain of rat IgG2c
GKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK